(19) United States
(12) Patent Application Publication
(10) Pub. No.: US 2014/0235536 A1
(43) Pub. Date: Aug. 21, 2014

(54) INJECTABLE SOLUTION AT PH 7 COMPRISING AT LEAST ONE BASAL INSULIN THE ISOELECTRIC POINT OF WHICH IS COMPRISED IN 5.8 AND 8.5 AND AN ANIONIC COMPOUND BEARING CARBOXYLATE CHARGES AND HYDROPHOBIC RADICALS

(71) Applicant: ADOCIA, Lyon (FR)

(72) Inventors: Olivier SOULA, Meyzieu (FR); Richard CHARVET, Rillieux la Pape (FR); Alexandre GEISSLER, Lyon (FR)

(73) Assignee: ADOCIA, Lyon (FR)

(21) Appl. No.: 14/179,212

(22) Filed: Feb. 12, 2014

Related U.S. Application Data
(60) Provisional application No. 61/763,766, filed on Feb. 12, 2013.

(30) Foreign Application Priority Data
Feb. 12, 2013 (FR) 13/51199

(51) Int. Cl. A61K 47/28; C07J 41/00; C07H 15/04; A61K 47/22; C07H 15/26; C07D 311/72; A61K 47/26; A61K 38/28; C07H 15/18

(52) U.S. Cl. CPC A61K 47/28; A61K 38/28; C07J 41/0055; C07H 15/04; C07H 15/18; C07H 15/26; C07D 311/72; A61K 47/26; A61K 47/22
USPC 514/5.9; 536/17.9; 552/544; 549/410

(57) ABSTRACT

A composition in the form of an injectable aqueous solution, the pH of which is from 6.6 to 7.8, comprises at least:
a) a basal insulin, the isoelectric point pI of which is between 5.8 and 8.5; and
b) an anionic compound bearing carboxylate charges and hydrophobic radicals.

The composition may also include a prandial insulin.

The invention relates to therapies by injection of insulin(s) for treating diabetes.

Insulin therapy, or therapy for diabetes by injection of insulin, has experienced remarkable progress over the past few years by virtue in particular of the development of new insulins which offer better correction of blood glucose level in patients in comparison with human insulin and which make it possible to simulate more closely the physiological activity of the pancreas.

When type II diabetes is diagnosed in a patient, a gradual treatment is put in place. The patient firstly takes oral antidiabetics (OADs) such as metformin. When OADs alone are no longer sufficient to regulate the glucose level in the blood, a change in the treatment must be made and, depending on the patients' specificities, various treatment combinations can be put in place. The patient can, for example, have a treatment based on a basal insulin of glargine or detemir type as a supplement to the OADs, then subsequently, depending on the progression of the disease, a treatment based on basal insulin and prandial insulin.

Moreover, today, in order to ensure the transition from treatments with OADs, when the latter are no longer able to control the glucose level in the blood, to a basal insulin/prandial insulin treatment, the injection of GLP-1 analogs is recommended.

GLPs-1, for Glucagon-Like Peptide-1, are insulinotropic peptides or incretins, and belong to the family of gut hormones which stimulate insulin secretion when the blood glucose level is too high, for example after a meal.

Gut hormones are also called satiating hormones. They comprise in particular GLP-1 (Glucagon like peptide-1) and GIP (Glucose-dependent insulinotropic peptide), oxyntomodulin (a proglucagon derivative), peptide YY, amylin, cholecystokinin, pancreatic polypeptide (PP), ghrelin and enterostatin which have peptide or protein structures. They also stimulate insulin secretion, in response to glucose and fatty acids, and are therefore in this respect potential candidates for the treatment of diabetes.

Among these gut hormones, GLPs-1 are those which have to date provided the best results in the development of medicaments. They have enabled patients suffering from type II diabetes to lose weight while at the same time having a better control of their blood glucose level.

GLP-1 analogs or derivatives have thus been developed, in particular for improving their stability.

To cover his daily insulin needs, a diabetic patient currently has, schematically, two types of insulins that have complementary actions: prandial insulins (or “fast-acting” insulins) and basal insulins (or “slow-acting” insulins).

The prandial insulins allow a rapid management (metabolization and/or storage) of the glucose taken in during meals and snacks. The patient must inject himself with a prandial insulin before each food intake, i.e. approximately 2 to 3 injections per day. The prandial insulins most widely used are: recombinant human insulin, NovoLog® (insulin aspart from NOVO NORDISK), Humalog® (insulin lispro from ELI LILLY) and Apidra® (insulin glulisine from SANOFI-AVENTIS).

The basal insulins maintain the glycemic homeostasis of the patient, outside periods of food intake. They act essentially to block the endogenous production of glucose (hepatic glucose). The daily dose of insulin generally corresponds to 40-50% of the total daily insulin needs. Depending on the basal insulin used, this dose is dispensed in 1 or 2 injections, spread out regularly over the course of the day. The basal insulins most widely used are Levemir® (insulin detemir from NOVO NORDISK) and Lantus® (insulin glargine from SANOFI-AVENTIS).

It will be noted, in the interest of being thorough, that NPH (insulin NPH for Neutral Protamine Hagedorn; Humuline NPH®, Insulatard®) is the oldest basal insulin. This formulation is the result of a precipitation of human insulin (anionic at neutral pH) using a cationic protein, protamine. The microcrystals thus formed are dispersed in an aqueous suspension and dissolve slowly after subcutaneous injection. This slow dissolution provides a prolonged release of the insulin. However, this release does not provide a constant concentration of insulin over time. The release profile is bell-shaped and only lasts between 12 and 16 hours. It is therefore injected twice a day. This NPH basal insulin is much less effective than the modern basal insulins, Levemir® and Lantus®. NPH is an intermediate-action basal insulin.

The principle of NPH has evolved with the appearance of the fast-acting insulin analogs to give products called “Premix” that offer both a fast action and an intermediate action. NovoLog Mix® (NOVO NORDISK) and Humalog Mix® (ELI LILLY) are formulations comprising a fast-acting insulin analog, Novolog® and Humalog®, partially complexed with protamine. These formulations thus contain insulin analog microcrystals, the action of which is termed intermediate, and an insulin component that has remained soluble, the action of which is fast. These formulations clearly offer the advantage of a fast-acting insulin, but they also have the defect of NPH, i.e. a limited duration of action of between 12 and 16 hours and an insulin with a “bell-shaped” release profile. However, these products allow patients to give themselves, in one go, an injection of an intermediate-action basal insulin with a fast-acting prandial insulin. As it happens, there are many patients who are anxious to reduce their number of injections.

The basal insulins currently marketed and currently in clinical development can be classified according to the technical solution which makes it possible to obtain the prolonged action, and, to date, two approaches are used.

The first approach, which is that of insulin detemir, is binding to albumin in vivo. Insulin detemir is an analog, which is soluble at pH 7, and which comprises a fatty acid (tetradecanoyl) side chain attached at position B29 which, in vivo, enables this insulin to associate with albumin. Its prolonged action is mainly due to this affinity for albumin after subcutaneous injection.

However, its pharmacokinetic profile does not make it possible to cover a day, which means that it is most commonly used as two injections per day.

Other basal insulins which are soluble at pH 7, such as Degludec®, are currently in development. Degludec® also comprises a fatty acid side chain attached to the insulin (hexadecanedioyl-γ-L-Glu).

The second approach, which is that of insulin glargine, is precipitation at physiological pH. Insulin glargine is a human insulin analog obtained by elongation of the C-terminal of the B chain of human insulin with two arginine residues, and by substitution of asparagine residue A21 with a glycine residue (U.S. Pat. No. 5,656,722). The addition of two arginine residues was considered in order to adjust the pI (isoelectric point) of insulin glargine at physiological pH, and thus to render this human insulin analog insoluble in physiological medium.

Also, the substitution of A21 was considered in order to render insulin glargine stable at acid pH and thus to be able to formulate it in the form of an injectable solution at acid pH. During subcutaneous injection, the passing of insulin glargine from an acid pH (pH 4-4.5) to a physiological pH (neutral pH) causes it to precipitate under the skin. The slow redissolution of the insulin glargine microparticles provides a slow and prolonged action.

The hypoglycemic effect of insulin glargine is virtually constant over a period of 24 hours, which enables most patients to limit themselves to a single injection per day.

Insulin glargine is today considered to be the best basal insulin on the market.

However, the necessarily acid pH of the formulations of basal insulins, the isoelectric point of which is between 5.8 and 8.5, of insulin glargine type, can be a real drawback since this acid pH of the insulin glargine formulation sometimes causes pain at the injection in patients and especially prevents any formulation with other proteins and in particular with prandial insulins, since the latter are not stable at acid pH. The impossibility of formulating a prandial insulin at acid pH comes from the fact that a prandial insulin undergoes, under these conditions, a side reaction consisting of deamidation in position A21, which does not make it possible to meet the requirement of the US pharmacopeia, namely less than 5% of by-products after 4 weeks at 30° C.

Thus, no one has to date sought to solubilize these basal insulins, of insulin glargine type, the isoelectric point of which is between 5.8 and 8.5, at neutral pH while at the same time maintaining a difference in solubility between the in vitro medium (the container) and the in vivo medium (under the skin), independently of the pH.

From the analysis of the compositions described in the literature and the patents, it appears that the insolubility at pH 7 of the basal insulins, of the insulin glargine type, is a prerequisite for having a slow action.

Indeed, the principle of how basal insulins, of insulin glargine type, the isoelectric point of which is between 5.8 and 8.5, function is that they are soluble at acid pH and precipitate at physiological pH. This diverts those skilled in the art from any solution in which the insulin of insulin glargine type would be solubilized at pH 6-8 while keeping its essential property which is that of precipitating in subcutaneous medium.

Furthermore, this acid pH of the formulations of basal insulins, the isoelectric point of which is between 5.8 and 8.5, of insulin glargine type, even prevents any extemporaneous combination with prandial insulins at neutral pH.

Indeed, a recent clinical study, presented at the 69th Scientific Sessions of the American Diabetes Association, New Orleans, La., Jun. 5-9, 2009, 0019-OR, made it possible to verify this limitation of the use of insulin glargine. A dose of insulin glargine and a dose of prandial insulin (in the case in point, insulin lispro) were mixed just before injection (E. Cengiz et al., 2010; Diabetes care—33(5): 1009-12). This experiment made it possible to demonstrate a significant delay in the pharmacokinetic and the pharmacodynamic profiles of the prandial insulin, possibly giving rise to postprandial hyperglycemia and to nocturnal hypoglycemia. This study clearly confirms the incompatibility of insulin glargine with the fast-acting insulins currently on the market.

Moreover, the instruction leaflet for Lantus®, the commercial product based on insulin glargine from the company SANOFI-AVENTIS, explicitly informs users not to mix with a solution of prandial insulin, whatever it may be, owing to the serious risk of modifying the pharmacokinetics and the pharmacodynamics of the insulin glargine and/or of the prandial insulin mixed together.

However, from a therapeutic point of view, it has been demonstrated, as illustrated hereinafter, that treatments combining either an insulin glargine and a prandial insulin, or an insulin glargine and a GLP-1 analog, are of real interest.

As regards the combination of an insulin glargine and a prandial insulin, clinical studies made public during the 70th annual scientific sessions of the American Diabetes Association (ADA) of 2010, abstract 2163-PO and abstract number 0001-LB, in particular those carried out by the company SANOFI-AVENTIS, showed that treatments which combine Lantus®, insulin glargine and a prandial insulin are much more effective than treatments based on products of the “Premix” type, Novolog Mix® or Humalog Mix®.

As regards the combination of an insulin glargine and a GLP-1 analog, the FDA (Food and Drug Administration) approved, in October 2011, the injection of exenatide (Byetta®, AMYLIN PHARMACEUTICALS, Inc and ELI LILLY and Company) as therapy supplementing insulin glargine for patients suffering from type II diabetes who are not able to achieve control of their blood glucose level with the basal insulin analog alone.

It so happens, owing to the fact that the very principle, set out above, of basal insulins, the isoelectric point of which is between 5.8 and 8.5, is that they are soluble at acid pH and precipitate at physiological pH, all the solutions proposed for combining them with other products, such as prandial insulins or GLP-1 analogs or derivatives, are based on tests for solubilization of the prandial insulins or GLP-1 analogs or derivatives at acid pH, see for example WO2007/121256, WO2009/021955, WO2011/144673, WO2011/147980 or else WO2009/063072.

For example, as regards the combinations of insulin glargine and fast-acting insulin, the company BIODEL has described, in particular in patent application U.S. Pat. No. 7,718,609, compositions comprising a basal insulin and a prandial insulin at a pH of between 3.0 and 4.2 in the presence of a chelating agent and of polyacids. This patent teaches how to make compatible a prandial insulin at acid pH in the presence of insulin glargine. It does not teach how to prepare a combination of insulin of insulin glargine type and of a prandial insulin at neutral pH.

Likewise by way of example, as regards the solubilization of insulin glargine at neutral pH and combinations with a GLP-1 analog, mention will be made of patent application WO2011/144676 published on Nov. 24, 2011, in the name of SANOFI-AVENTIS, which describes formulations, at pH 9.5, of insulin glargine with the cyclodextrin SVE4-β-CYD in which the solubility of insulin glargine is improved from 0.75 mM to 1.25 mM. This application also mentions compositions additionally comprising a GLP-1, although they are not exemplified. The solubilizing effect at pH 7.4 in a phosphate buffer is mentioned. These results of solubilization at pH 7.4 are described in the publication entitled “Effect of sulfobutyl ether-β-cyclodextrin on bioavailability of insulin glargine and blood glucose level after subcutaneous injection to rats” (International Journal of Pharmaceutics, 419 (2011), 71-76) in FIG. 3A. The sulfobutyl ether-β-cyclodextrin improves the solubility of the insulin glargine at pH 7.4 from 5 μM to 8 μM, which is of no therapeutic interest, since the commercial concentration of insulin glargine is 600 μM (100 IU/ml). The problem has thus not been satisfactorily solved by the invention described in this patent application.

To our knowledge, a formulation which is stable at physiological pH, comprising a basal insulin, the isoelectric point of which is between 5.8 and 8.5, alone or in combination with a prandial insulin and/or a gut hormone, in which the solubility of the insulin is sufficient for a therapeutic treatment, has therefore never been described.

The present invention, by solving this problem of solubility at a pH between 6.6 and 7.8, makes it possible:
to propose an injectable composition, intended for the treatment of diabetes, comprising a basal insulin, the isoelectric point of which is between 5.8 and 8.5, in the form of a homogeneous solution at a pH of between 6.6 and 7.8, while at the same time retaining its biological activity and its slow action profile;
to propose an injectable composition in the form of a homogeneous solution at a pH of between 6.6 and 7.8, also comprising a combination of a basal insulin, the isoelectric point of which is between 5.8 and 8.5, and of a prandial insulin without modification of the activity profile of the prandial insulin which is soluble at pH 6-8 and unstable at acid pH, while at the same time maintaining the slow action profile specific to the basal insulin;
to propose an injectable composition in the form of a homogeneous solution at a pH of between 6.6 and 7.8, also comprising a combination of a basal insulin, the isoelectric point of which is between 5.8 and 8.5, and of a gut hormone derivative or analog, such as GLP-1 or glucagon like peptide-1;
to reduce the number of injections in the context of the treatment of diabetes;
for said compositions to comply with the requirements of the US and European Pharmacopeias.

Surprisingly, the compositions according to the invention the invention make it possible to solubilize, at a pH between 6.6 and 7.8, a basal insulin, the isoelectric point of which is between 5.8 and 8.5.

Surprisingly, the compositions according to the invention make it possible to maintain the duration of the hypoglycemic activity of the basal insulin, the isoelectric point of which is between 5.8 and 8.5, despite its solubilization at a pH of between 6.6 and 7.8 before injection. This notable property comes from the fact that the insulin of insulin glargine type solubilized at a pH of between 6.6 and 7.8 in the composition of the invention precipitates in subcutaneous medium through a change in composition of the medium. The element which triggers the precipitation of the insulin of insulin glargine type is no longer the pH modification, but a modification of the composition of the environment when the pharmaceutical composition passes from the container to the physiological medium. Surprisingly, in the combinations of insulin of insulin glargine type with a prandial insulin, which are subjects of the invention, the fast action of the prandial insulin is preserved despite the precipitation of the insulin of insulin glargine type in subcutaneous medium.

The solution as claimed in the invention making it possible to solubilize the basal insulin, the isoelectric point of which is between 5.8 and 8.5, at a pH of between 6.6 and 7.8, preserves its biological activity.

In the combinations of the insulin of insulin glargine type with a prandial insulin which are subjects of the invention, the fast action of the prandial insulin is preserved despite the precipitation of the insulin of insulin glargine type in subcutaneous medium. Furthermore, the presence of the prandial insulin does not modify the solubility of the basal insulin at a pH of between 6.6 and 7.8 and likewise does not modify the precipitation properties of the basal insulin.

The invention relates to a composition in the form of an injectable aqueous solution, the pH of which is between 6.6 and 7.8, comprising at least:
a) a basal insulin, the isoelectric point pI of which is between 5.8 and 8.5;
b) an anionic compound bearing carboxylate charges and hydrophobic radicals, of formula I:

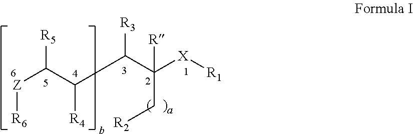

in which:
1) a=0 or 1, and
2) b=0 or 1, and
3) —X— is either a —C═O— radical, or a —CH₂— radical; and
4) —Z— is either a —C═O— radical, or a —CH₂— radical; and
5) —R₁ is chosen from the group consisting of the radicals:
   i. —OH;
   ii. -f-[A]-COOH, then —X—═—CH₂—;
   in which,
      -A- is an at least divalent radical comprising from 1 to 15 carbon atoms comprising at least one heteroatom chosen from O, N and S, optionally bearing carboxyl or amine functions and/or -f-[A]-COOH, comprising from 2 to 16 carbon atoms, is derived from an amino acid, from a diacid or from an alcohol acid and is bonded to the backbone of the molecule via a function f;
      f is chosen from the group consisting of ether, ester, carbamate, amide or carbonate functions;
   iii. -g-[B]-(k-[D])ₚ, then —X—═—CH₂—;
   in which,
      —B— is an at least divalent radical comprising from 1 to 15 carbon atoms comprising at least one heteroatom chosen from O, N and S, optionally bearing carboxyl or amine functions and/or -g-[B]-(k-)ₚ, comprising from 2 to 16 carbon atoms, is derived from an amino acid, from a diacid, from a dialcohol, from an alcohol acid, from a diamine or from an amine alcohol and is bonded to the backbone of the molecule via a function g and is bonded to at least one radical -D via a function k;
      g is chosen from the group consisting of ether, ester, carbamate, amide or carbonate functions;
      k is chosen from the group consisting of ester, amide, carbamate or carbonate functions;
      p is a positive integer equal to 1 or 2;
      said radical -D being a radical -[Hy] if p=2 or -[E]-(o-[Hy])ₜ if p=1; in which,
      -E- is an at least divalent radical comprising from 1 to 15 carbon atoms comprising at least one heteroatom chosen from O, N and S, optionally bearing carboxyl or amine functions and/or k-[E]-(o-)ₜ, comprising from 2 to 16 carbon atoms, is derived from an amino acid, from a dialcohol, from a diamine, from a diacid or from an amine alcohol;
      -Hy is a C₈ to C₃₀ linear or cyclic alkyl group or a C₈ to C₃₀ alkylaryl or arylalkyl, optionally substituted with one or more C₁ to C₃ alkyl groups, derived from a hydrophobic compound;
      o is an ester, amide, carbamate or carbonate function;
      t being a positive integer equal to 1 or 2;
      k and o being identical or different;
   iv. —NH-[E]-(o-[Hy])ₜ if —R′, —R₂, —R₃, —R₄, —R₅ and —R₆ are different from
      -g-[B]-k-[D], —NH-[D] and —O-[D], in which
      -E-, -Hy, o and t have the definitions given above, and
   v. —N(L)z-[E]-(o-[Hy])ₜ if —R′, —R₂, —R₃, —R₄, —R₅ and —R₆ are different from
      -g-[B]-k-[D], —NH-[D] and —O-[D], and if —X—═—CH₂—, in which,
      -D-, -Hy, -E-, o and t have the definitions given above;
      z is equal to 1 or 2;
      -L is chosen from the group consisting of:
         —H then z is equal to 1, and/or
         -[A]-COOH if f is an ether function,
         —CO-[A]-COOH and z is equal to 1, if f is an ester function, and
         —CO—NH-[A]-COOH and z is equal to 1 if f is a carbamate function;
         A has the meaning given above;
   vi. -A-, —B— or -E- identical or different;
6) —R″ is chosen from the group consisting of the radicals:
   i. —H, hydrogen atom, if b=1;
   ii. -k-[Hy] or -k-[E]-(o-[Hy])ₜ if b=0, a=0 and —R₁, —R₂, —R₃ different from -g-[B]-(k-[D])ₚ and —NH-[D];
      -E- and t have the definitions given above;
      -Hy is a C₁₂ to C₃₀ linear or cyclic alkyl group or a C₁₂ to C₃₀ alkylaryl or arylalkyl, optionally substituted with one or more C₁ to C₃ alkyl groups, derived from a hydrophobic compound;
7) —R₅ is chosen from the group consisting of the radicals:
   i. —OH; or
   ii. -f-[A]-COOH; or
   iii. -g-[B]-(k-[D])ₚ;
      in which:
         -A-, —B—, -D- and p are defined as above,
         f, g and k, which may be identical or different, are defined as above;
8) —R₃ or —R₄, which may be identical or different, are chosen from the group consisting of the radicals:
   i. —OH; or
   ii. -f-[A]-COOH; or
   iii. -g-[B]-(k-[D])ₚ; or
      -A-, —B—, -D-, p, f, g and k being defined as above;
   and/or
      —R₆ is chosen from the group consisting of the radicals:
   i. —OH; or
   ii. -f-[A]-COOH if —Z—═—CH₂—; or
   iii. -g-[B]-(k-[D])ₚ if —Z—═—CH₂—; or
   iv. —O-[D] if —Z—═—C═O—;
   v. —NH-[D] if —Z—═—C═O—;
      -A-, —B—, -D-, p, f, g and k being defined as above;
         and/or
—R₂ is chosen from the group consisting of the radicals:
   i. —OH; or
   ii. -f-[A]-COOH; or
   iii. -g-[B]-(k-[D])ₚ; or
   iv. —NH—COCH₃; or
   v. —NH₂;
   vi. —NH-[D];
      -A-, —B—, -D-, p, f, g and k being defined as above;
         and/or
at most one of —R₂, —R₃, —R₄ and —R₆ is a backbone formed from a discrete number u of between 1 and 7 (1≦u≦7) of identical or different saccharide units linked via identical or different glycosidic linkages, said saccharide units being chosen from the group consisting of:
   i. hexoses in cyclic form or in open reduced form, uronic acids in cyclic form or in open oxidized form,
   iii. hexosamines in cyclic form, in open reduced form or in open oxidized form,
   iv. N-acetylhexosamines in cyclic form or in open reduced form, and
at least one of said saccharide units is substituted with at least one substituent —R′ chosen from the group consisting of:
   -f-[A]-COOH and/or
   -k-[D] or -g-[B]-(k-[D])ₚ, in which
   -A-, —B—, -D-, p, f, g and k are defined as above; and
   when —R₁ is —NH-[E]-(o-[Hy])ₜ or —N(L)_{z}-[E]-(o-[Hy])ₜ then —R₂, —R₃, —R₄, —R₅ and —R₆ are different from -g-[B]-(k-[D])ₚ, —O-[D] or —NH-[D] and —R′ is different from -k-[D] or -g-[B]-(k-[D])ₚ;
and
9) the asymmetric carbon atoms are of absolute configuration R or S; the free acid functions being in the form of salts of alkali metal cations chosen from the group consisting of Na⁺ and K⁺.

In one embodiment, the anionic compound bearing carboxylate charges and hydrophobic radicals of formula I is in the isolated state or as a mixture.

The term “hydrophobic” radical or group is intended to mean a radical or a group derived from a hydrophobic compound.

The term “hydrophobic compound” is intended to mean a compound which has a Log P greater than or equal to 2. The Log P or Log Kow or partition coefficient is a measurement of the distribution of a compound in a mixture of n-octanol immiscible solvent/water. The Log P can be measured according to the shake flask method, or when this is not possible, by the HPLC method (OECD Guideline for the testing of chemicals, 117, Mar. 30, 1989, Partition coefficient (n-octanol/water): HPLC method and 107, Jul. 27, 1995, Partition coefficient (n-octanol/water): Shake Flask Method). Said Log P of a compound being defined by the equation:

log P=log(C_{oct}/C_{water})

in which C_{oct} is the concentration of said compound in n-octanol and C_{water} is the concentration of said compound in water.

In one embodiment, the composition according to the invention is characterized in that the anionic compound bearing carboxylate charges and hydrophobic radicals is chosen from the compounds of formula I in which the radical -f-[A]-COOH is chosen from the radicals of formula VI below:

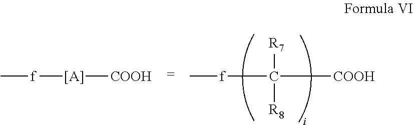

in which:
i is greater than or equal to 1 and less than or equal to 12, and
R₇ and R₈, which may be identical or different, are chosen from the group consisting of a hydrogen atom, a saturated or unsaturated, linear, branched or cyclic C₁ to C₆ alkyl, a benzyl, an alkylaryl, optionally comprising heteroatoms chosen from the group consisting of O, N and/or S, or functions chosen from the group consisting of carboxylic acid, amine, alcohol and thiol functions,
-f-[A]-COOH comprises from 2 to 16 carbon atoms.

In one embodiment, the composition according to the invention is characterized in that the anionic compound bearing carboxylate charges and hydrophobic radicals is chosen from the compounds of formula I in which the radical -f-[A]-COOH, comprising from 2 to 8 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula I in which the radical -f-[A]-COOH, comprising from 2 to 6 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula I in which the radical -f-[A]-COOH is chosen from the group consisting of the following sequences, f having the meaning given above:

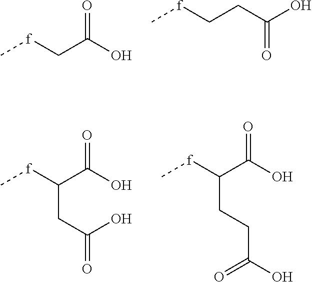

or the salts thereof with alkali metal cations chosen from the group consisting of Na⁺ and K⁺.

In one embodiment, the composition according to the invention is characterized in that the anionic compound bearing carboxylate charges and hydrophobic radicals is chosen from the compounds of formula I in which the radical -f-[A]-COOH is -f-CH₂—COOH.

In one embodiment, the composition according to the invention is characterized in that the anionic compound bearing carboxylate charges and hydrophobic radicals is chosen from the compounds of formula I in which the radical -f-[A]-COOH is derived from an alpha amino acid.

In one embodiment, the composition according to the invention is characterized in that the anionic compound bearing carboxylate charges and hydrophobic radicals is chosen from the compounds of formula I in which the radical -f-[A]-COOH is derived from glycine.

In one embodiment, the composition according to the invention is characterized in that the anionic compound bearing carboxylate charges and hydrophobic radicals is chosen from the compounds of formula I in which the radical -f-[A]-COOH is derived from succinic acid.

In one embodiment, the composition according to the invention is characterized in that the anionic compound bearing carboxylate charges and hydrophobic radicals is chosen from the compounds of formula I in which the radical -f-[A]-COOH is derived from aspartic acid.

In one embodiment, the composition according to the invention is characterized in that the anionic compound bearing carboxylate charges and hydrophobic radicals is chosen from the compounds of formula I in which the radical -f-[A]-COOH is derived from glutamic acid.

In one embodiment, the composition according to the invention is characterized in that the anionic compound bearing carboxylate charges and hydrophobic radicals is chosen from the compounds of formula I in which the radical -f-[A]-COOH is derived from a beta amino acid.

In one embodiment, the composition according to the invention is characterized in that the anionic compound bearing carboxylate charges and hydrophobic radicals is chosen from the compounds of formula I in which the radical -f-[A]-COOH is derived from β-alanine.

In one embodiment, the composition according to the invention is characterized in that the anionic compound bearing carboxylate charges and hydrophobic radicals is chosen from the compounds of formula I in which the function f is an ether function.

In one embodiment, the composition according to the invention is characterized in that the anionic compound bearing carboxylate charges and hydrophobic radicals is chosen from the compounds of formula I in which the function f is a carbamate function.

In one embodiment, the composition according to the invention is characterized in that the anionic compound bearing carboxylate charges and hydrophobic radicals is chosen from the compounds of formula I in which the function f is an ester function.

In one embodiment, the composition according to the invention is characterized in that the anionic compound bearing carboxylate charges and hydrophobic radicals is chosen from the compounds of formula I in which the function f is a carbonate function.

In one embodiment, the composition according to the invention is characterized in that the anionic compound bearing carboxylate charges and hydrophobic radicals is chosen from the compounds of formula I in which the function f is an amide function.

In one embodiment, the composition according to the invention is characterized in that the anionic compound bearing carboxylate charges and hydrophobic radicals is chosen from the compounds of formula I in which a=1, b=0 and —X— is a radical —CH₂—.

In one embodiment, the composition according to the invention is characterized in that the anionic compound bearing carboxylate charges and hydrophobic radicals is chosen from the compounds of formula I, in which a=1, b=0 and —X— is a radical —CH₂—, is a compound chosen from the compounds of formula II:

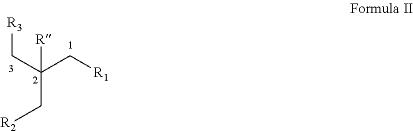

in which:
1) —R₁, —R₂ and —R₃, which may be identical or different, are radicals -f-[A]-COOH,
2) —R″ is either -k-[Hy], or -k-[E]-(o-[Hy])ₜ,
3) -A-, -E-, t, -Hy, f, k and o are as defined above,
4) f, k and o being identical or different,
5) the free acid functions being in the form of salts of alkali metal cations chosen from the group consisting of Na+ and K+.

In one embodiment, the composition according to the invention is characterized in that the anionic compound bearing carboxylate charges and hydrophobic radicals is chosen from the compounds of formula II in which the radical —R″ is a radical -k-[Hy], k and -Hy having the definitions given above.

In one embodiment, the composition according to the invention is characterized in that the anionic compound bearing carboxylate charges and hydrophobic radicals is chosen from the compounds of formula II in which the radical —R″ is a radical -k-[E]-(o-[Hy])ₜ, k, o, t, -E- and -Hy having the definitions given above.

In one embodiment, the composition according to the invention is characterized in that the anionic compound bearing carboxylate charges and hydrophobic radicals is chosen from the compounds of formula II in which the radical -k-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula II in which the radical -k-E-(o-)ₜ is an at least divalent radical derived from an amino acid.

In one embodiment, the composition according to the invention is characterized in that the anionic compound bearing carboxylate charges and hydrophobic radicals is chosen from the compounds of formula II in which the radical -k-E-(o-)ₜ is an at least divalent radical derived from an alpha amino acid.

In one embodiment, the composition according to the invention is characterized in that the anionic compound is such that the radical -k-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula II in which the radical -k-E-(o-)ₜ is an at least divalent radical derived from a beta amino acid.

In one embodiment, the composition according to the invention is characterized in that the anionic compound is chosen from the compounds of formula II in which the at least divalent radical -k-E-(o-)ₜ, derived from a beta amino acid, is β-alanine.

In one embodiment, the composition according to the invention is characterized in that the anionic compound is chosen from the compounds of formula II in which the radical -k-E-(o-)ₜ is an at least divalent radical derived from a mono- or polyethylene glycol.

In one embodiment, the composition according to the invention is characterized in that the anionic compound is chosen from the compounds of formula II in which the radical -k-E-(o-)ₜ is an at least divalent radical derived from ethylene glycol.

In one embodiment, the composition according to the invention is characterized in that the anionic compound is chosen from the compounds of formula II in which the radical -k-E-(o-)ₜ is an at least divalent radical derived from a polyethylene glycol chosen from the group consisting of diethylene glycol, triethylene glycol and tetraethylene glycol.

In one embodiment, the composition according to the invention is characterized in that the anionic compound is chosen from the compounds of formula II in which the radical -k-E-(o-)ₜ is an at least divalent radical derived from a mono- or polyethylene glycol amine.

In one embodiment, the composition according to the invention is characterized in that the anionic compound is chosen from the compounds of formula II in which the radical -k-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound is chosen from the compounds of formula II in which the radical -k-E-(o-)ₜ is an at least divalent radical derived from a mono- or polyethylene glycol diamine.

In one embodiment, the composition according to the invention is characterized in that the anionic compound is chosen from the compounds of formula II in which the radical -k-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound is chosen from the compounds of formula II in which the radical -k-E-(o-)ₜ is an at least divalent radical derived from ethylenediamine.

In one embodiment, the composition according to the invention is characterized in that the anionic compound is chosen from the compounds of formula II in which the function o is an ester function.

In one embodiment, the composition according to the invention is characterized in that the anionic compound is chosen from the compounds of formula II in which the function o is an amide function.

In one embodiment, the composition according to the invention is characterized in that the anionic compound is chosen from the compounds of formula II in which the function o is a carbamate function.

In one embodiment, the composition according to the invention is characterized in that the anionic compound is chosen from the compounds of formula II in which the function o is a carbonate function.

In one embodiment, the composition according to the invention is characterized in that the anionic compound is chosen from the compounds of formula II in which the -Hy group is an alkyl group derived from a branched or unbranched, unsaturated and/or saturated, hydrophobic alcohol comprising from 12 to 30 carbons.

In one embodiment, the composition according to the invention is characterized in that the anionic compound is chosen from the compounds of formula II in which the -Hy group is an alkyl group derived from a hydrophobic alcohol chosen from the group consisting of dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound is chosen from the compounds of formula II in which the -Hy group is a group derived from a sterol.

In one embodiment, the composition according to the invention is characterized in that the anionic compound is chosen from the compounds of formula II in which the -Hy group is a group derived from a sterol, chosen from the group consisting of cholesterol and derivatives thereof.

In one embodiment, the composition according to the invention is characterized in that the anionic compound is chosen from the compounds of formula II in which the -Hy group is a group derived from cholesterol.

In one embodiment, the composition according to the invention is characterized in that the anionic compound is chosen from the compounds of formula II in which the -Hy group is a group derived from a tocopherol.

In one embodiment, the composition according to the invention is characterized in that the anionic compound is chosen from the compounds of formula II in which the -Hy group is a group derived from a tocopherol derivative, chosen from the racemate, the L isomer or the D isomer of α-tocopherol.

In one embodiment, the composition according to the invention is characterized in that the anionic compound is chosen from the compounds of formula II in which the -Hy group is a group derived from DL-α-tocopherol.

In one embodiment, the composition according to the invention is characterized in that the anionic compound is chosen from the compounds of formula II in which the -Hy group is an alkyl group derived from a hydrophobic acid.

In one embodiment, the composition according to the invention is characterized in that the anionic compound is chosen from the compounds of formula II in which the -Hy group is an alkyl group derived from a fatty acid.

In one embodiment, the composition according to the invention is characterized in that the anionic compound is chosen from the compounds of formula II in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the composition according to the invention is characterized in that the anionic compound is chosen from the compounds of formula II in which the -Hy group is an alkyl group derived from a fatty acid chosen from the group consisting of linear fatty acids.

In one embodiment, the composition according to the invention is characterized in that the anionic compound is chosen from the compounds of formula II in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound is chosen from the compounds of formula II in which the -Hy group is an alkyl group derived from an unsaturated fatty acid.

In one embodiment, the composition according to the invention is characterized in that the anionic compound is chosen from the compounds of formula II in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound is chosen from the compounds of formula II in which the -Hy group is an alkyl group derived from a bile acid and derivatives thereof.

In one embodiment, the composition according to the invention is characterized in that the anionic compound is chosen from the compounds of formula II in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound is chosen from the compounds of formula II in which the radical —R″ is a radical -k-[Hy], such that k is a carbamate function and the -Hy group is derived from cholesterol.

In one embodiment, the composition according to the invention is characterized in that the anionic compound is chosen from the compounds of formula II in which the radical —R″ is a radical -k-[Hy], such that k is a carbamate function and the -Hy group is derived from α-tocopherol.

In one embodiment, the composition according to the invention is characterized in that the anionic compound is chosen from the compounds of formula II in which the radical —R″ is a radical -k-[Hy], such that k is a carbamate function and the -Hy group is derived from cholesterol, f is a carbamate function, and -f-[A]-COOH is derived from glycine.

In one embodiment, the composition according to the invention is characterized in that the anionic compound is chosen from the compounds of formula II in which the radical —R″ is a radical -k-[Hy], such that k is a carbamate function and the -Hy group is derived from cholesterol, f is a carbamate function, and -f-[A]-COOH is derived from aspartic acid.

In one embodiment, the composition according to the invention is characterized in that the anionic compound is chosen from the compounds of formula II in which the radical —R″ is a radical -k-[Hy], such that k is a carbamate function and the -Hy group is derived from cholesterol, f is a carbamate function, and -f-[A]-COOH is derived from glutamic acid.

In one embodiment, the composition according to the invention is characterized in that the anionic compound is chosen from the compounds of formula II in which the radical —R″ is a radical -k-[Hy], such that k is a carbamate function and the -Hy group is derived from α-tocopherol, f is a carbamate function, and -f-[A]-COOH is derived from glycine.

In one embodiment, the composition according to the invention is characterized in that the backbone before substitution with radicals bearing carboxylate charges and hydrophobic radicals is TRIS.

The invention also relates to an anionic compound bearing carboxylate charges and hydrophobic radicals chosen from the compounds of formula I, in which a=1, b=0 and —X— is a radical —CH₂—:

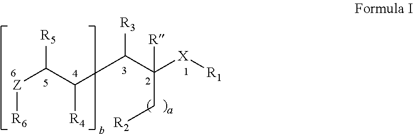

in which:
1) a=1, and
2) b=0, and
3) —X— is a radical —CH₂—; and
4) —R₁ is chosen from the group consisting of the radicals:
   i. —OH;
   ii. -f-[A]-COOH;
   in which,
      -A- is an at least divalent radical comprising from 1 to 15 carbon atoms comprising at least one heteroatom chosen from O, N and S, optionally bearing carboxyl or amine functions and/or -f-[A]-COOH, comprising from 2 to 16 carbon atoms, is derived from an amino acid, from a diacid or from an alcohol acid and is bonded to the backbone of the molecule via a function f;
      f is chosen from the group consisting of ether, ester, carbamate, amide or carbonate functions;
5) —R″ is chosen from the group consisting of the radicals:
   i. -k-[Hy] or -k-[E]-(o-[Hy])ₜ
      k is chosen from the group consisting of ester, amide, carbamate or carbonate functions;
      -E- is an at least divalent radical comprising from 1 to 15 carbon atoms comprising at least one heteroatom chosen from O, N and S, optionally bearing carboxyl or amine functions and/or k-[E]-(o-)ₜ, comprising from 2 to 16 carbon atoms, is derived from an amino acid, from a dialcohol, from a diamine, from a diacid or from an amine alcohol;
      o is an ester, amide, carbamate or carbonate function; and
      t being a positive integer equal to 1 or 2;
      k and o being identical or different;
      -Hy is a C₁₂ to C₃₀ linear or cyclic alkyl group or a C₁₂ to C₃₀ alkylaryl or arylalkyl, optionally substituted with one or more C₁ to C₃ alkyl groups, which is derived from a hydrophobic compound;
6) —R₂ and —R₃ are chosen from the group consisting of the radicals:
   iii. —OH; or
   iv. -f-[A]-COOH; or
      -A- and f being defined as above;
      and
7) the assymetrical carbon atoms are of absolute configuration R or S; the free acid functions being in the form of salts of alkali metal cations chosen from the group consisting of Na⁺ and K⁺.

In one embodiment, the anionic compound bearing carboxylate charges and hydrophobic radicals is chosen from the compounds of formula I in which the radical -f-[A]-COOH is chosen from the radicals of formula VI below:

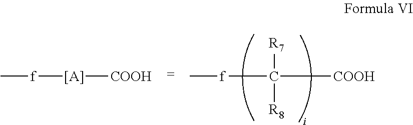

in which:
i is greater than or equal to 1 and less than or equal to 12, and
R₇ and R₈, which may be identical or different, are chosen from the group consisting of a hydrogen atom, a saturated or unsaturated, linear, branched or cyclic C₁ to C₆ alkyl, a benzyl, an alkylaryl, optionally comprising heteroatoms chosen from the group consisting of O, N and/or S, or functions chosen from the group consisting of carboxylic acid, amine, alcohol and thiol functions,
-f-[A]-COOH comprises from 2 to 16 carbon atoms.

In one embodiment, the anionic compound bearing carboxylate charges and hydrophobic radicals is chosen from the compounds of formula I in which the radical -f-[A]-COOH, comprising from 2 to 8 carbon atoms, is derived from an amino acid, from a dialcohol, from a diamine, from a diacid or from an amine alcohol.

In one embodiment, the anionic compound bearing carboxylate charges and hydrophobic radicals is chosen from the compounds of formula I in which the radical -f-[A]-COOH, comprising from 2 to 6 carbon atoms, is derived from an amino acid, from a dialcohol, from a diamine, from a diacid or from an amine alcohol.

In one embodiment, the anionic compound bearing carboxylate charges and hydrophobic radicals is chosen from the compounds of formula I in which the radical -f-[A]-COOH is chosen from the group consisting of the following sequences, f having the meaning given above:

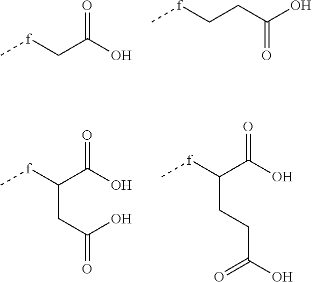

or the salts thereof with alkali metal cations chosen from the group consisting of Na⁺ and K⁺.

In one embodiment, the anionic compound bearing carboxylate charges and hydrophobic radicals is chosen from the compounds of formula I in which the radical -f-[A]-COOH is -f-CH₂—COOH.

In one embodiment, the anionic compound bearing carboxylate charges and hydrophobic radicals is chosen from the compounds of formula I in which the radical -f-[A]-COOH is derived from an alpha amino acid.

In one embodiment, the anionic compound bearing carboxylate charges and hydrophobic radicals is chosen from the compounds of formula I in which the radical -f-[A]-COOH is derived from glycine.

In one embodiment, the anionic compound bearing carboxylate charges and hydrophobic radicals is chosen from the compounds of formula I in which the radical -f-[A]-COOH is derived from succinic acid.

In one embodiment, the anionic compound bearing carboxylate charges and hydrophobic radicals is chosen from the compounds of formula I in which the radical -f-[A]-COOH is derived from aspartic acid.

In one embodiment, the anionic compound bearing carboxylate charges and hydrophobic radicals is chosen from the compounds of formula I in which the radical -f-[A]-COOH is derived from glutamic acid.

In one embodiment, the anionic compound bearing carboxylate charges and hydrophobic radicals is chosen from the compounds of formula I in which the radical -f-[A]-COOH is derived from a beta amino acid.

In one embodiment, the anionic compound bearing carboxylate charges and hydrophobic radicals is chosen from the compounds of formula I in which the radical -f-[A]-COOH is derived from β-alanine.

In one embodiment, the anionic compound bearing carboxylate charges and hydrophobic radicals is chosen from the compounds of formula I in which the function f is an ether function.

In one embodiment, the anionic compound bearing carboxylate charges and hydrophobic radicals is chosen from the compounds of formula I in which the function f is a carbamate function.

In one embodiment, the anionic compound bearing carboxylate charges and hydrophobic radicals is chosen from the compounds of formula I in which the function f is an ester function.

In one embodiment, the anionic compound bearing carboxylate charges and hydrophobic radicals is chosen from the compounds of formula I in which the function f is a carbonate function.

In one embodiment, the anionic compound bearing carboxylate charges and hydrophobic radicals is chosen from the compounds of formula I in which the function f is an amide function.

In one embodiment, the anionic compound bearing carboxylate charges and hydrophobic radicals of formula I, in which a=1, b=0 and —X— is a radical —CH₂—, according to the invention, is a compound chosen from the compounds of formula II:

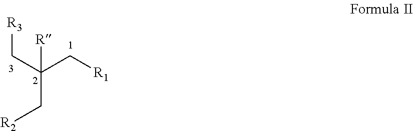

in which:
1) —R₁, —R₂ and —R₃, which may be identical or different, are radicals -f-[A]-COOH,
2) —R″ is either -k-[Hy], or -k-[E]-(o-[Hy])ₜ,
3) -A-, -E-, t, -Hy, f, k and o are as defined above,
4) f, k and o being identical or different,
5) the free acid functions being in the form of salts of alkali metal cations chosen from the group consisting of Na+ and K+.

In one embodiment, the anionic compound bearing carboxylate charges and hydrophobic radicals according to the invention, is chosen from the compounds of formula II in which the radical —R″ is a radical -k-[Hy], k and -Hy having the definitions given above.

In one embodiment, the anionic compound bearing carboxylate charges and hydrophobic radicals according to the invention, is chosen from the compounds of formula II in which the radical —R″ is a radical -k-[E]-(o-[Hy])ₜ, k, o, t, -E- and -Hy having the definitions given above.

In one embodiment, the composition according to the invention is characterized in that the anionic compound bearing carboxylate charges and hydrophobic radicals is chosen from the compounds of formula II in which the radical -k-E-(o-)ₜ is an at least divalent radical, comprising from 2 to 9 carbon atoms, is derived from an amino acid, from a dialcohol, from a diamine, from a diacid or from an amine alcohol.

In one embodiment, the anionic compound bearing carboxylate charges and hydrophobic radicals according to the invention, is chosen from the compounds of formula II in which the radical -k-E-(o-)ₜ is an at least divalent radical derived from an amino acid.

In one embodiment, the anionic compound bearing carboxylate charges and hydrophobic radicals according to the invention is chosen from the compounds of formula II in which the radical -k-E-(o-)ₜ is an at least divalent radical derived from an alpha amino acid.

In one embodiment, the anionic compound bearing carboxylate charges and hydrophobic radicals according to the invention is such that the radical -k-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the anionic compound bearing carboxylate charges and hydrophobic radicals according to the invention is chosen from the compounds of formula II in which the radical -k-E-(o-)ₜ is an at least divalent radical derived from a beta amino acid.

In one embodiment, the anionic compound bearing carboxylate charges and hydrophobic radicals according to the invention is chosen from the compounds of formula II in which the at least divalent radical -k-E-(o-)ₜ, derived from a beta amino acid, is β-alanine.

In one embodiment, the anionic compound bearing carboxylate charges and hydrophobic radicals according to the invention is chosen from the compounds of formula II in which the radical -k-E-(o-)ₜ is an at least divalent radical derived from a mono- or polyethylene glycol.

In one embodiment, the anionic compound bearing carboxylate charges and hydrophobic radicals according to the invention is chosen from the compounds of formula II in which the radical -k-E-(o-)ₜ is an at least divalent radical derived from ethylene glycol.

In one embodiment, the anionic compound bearing carboxylate charges and hydrophobic radicals according to the invention is chosen from the compounds of formula II in which the radical -k-E-(o-)ₜ is an at least divalent radical derived from a polyethylene glycol chosen from the group consisting of diethylene glycol, triethylene glycol and tetraethylene glycol.

In one embodiment, the anionic compound bearing carboxylate charges and hydrophobic radicals according to the invention is chosen from the compounds of formula II in which the radical -k-E-(o-)ₜ is an at least divalent radical derived from a mono- or polyethylene glycol amine.

In one embodiment, the anionic compound bearing carboxylate charges and hydrophobic radicals according to the invention is chosen from the compounds of formula II in which the radical -k-E-(o-)ₜ is an at least divalent radical derived from a mono- or polyethylene glycol amine chosen from the group consisting of ethanolamine, diethylene glycol amine and triethylene glycol amine.

In one embodiment, the anionic compound bearing carboxylate charges and hydrophobic radicals according to the invention is chosen from the compounds of formula II in which the radical -k-E-(o-)ₜ is an at least divalent radical derived from a mono- or polyethylene glycol diamine.

In one embodiment, the anionic compound bearing carboxylate charges and hydrophobic radicals according to the invention is chosen from the compounds of formula II in which the radical -k-E-(o-)ₜ is an at least divalent radical derived from a mono- or polyethylene glycol diamine chosen from the group consisting of diethylene glycol diamine and triethylene glycol diamine.

In one embodiment, the anionic compound bearing carboxylate charges and hydrophobic radicals according to the invention is chosen from the compounds of formula II in which the radical -k-E-(o-)ₜ is an at least divalent radical derived from ethylenediamine.

In one embodiment, the anionic compound bearing carboxylate charges and hydrophobic radicals according to the invention is chosen from the compounds of formula II in which the function o is an ester function.

In one embodiment, the anionic compound bearing carboxylate charges and hydrophobic radicals according to the invention is chosen from the compounds of formula II in which the function o is an amide function.

In one embodiment, the anionic compound bearing carboxylate charges and hydrophobic radicals according to the invention is chosen from the compounds of formula II in which the function o is a carbamate function.

In one embodiment, the anionic compound bearing carboxylate charges and hydrophobic radicals according to the invention is chosen from the compounds of formula II in which the function o is a carbonate function.

In one embodiment, the anionic compound bearing carboxylate charges and hydrophobic radicals according to the invention is chosen from the compounds of formula II in which the -Hy group is an alkyl group derived from a branched or unbranched, unsaturated and/or saturated, hydrophobic alcohol comprising from 12 to 30 carbons.

In one embodiment, the anionic compound bearing carboxylate charges and hydrophobic radicals according to the invention is chosen from the compounds of formula II in which the -Hy group is an alkyl group derived from a hydrophobic alcohol chosen from the group consisting of dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the anionic compound bearing carboxylate charges and hydrophobic radicals according to the invention is chosen from the compounds of formula II in which the -Hy group is a group derived from a sterol.

In one embodiment, the anionic compound bearing carboxylate charges and hydrophobic radicals according to the invention is chosen from the compounds of formula II in which the -Hy group is a group derived from a sterol, chosen from the group consisting of cholesterol and derivatives thereof.

In one embodiment, the anionic compound bearing carboxylate charges and hydrophobic radicals according to the invention is chosen from the compounds of formula II in which the -Hy group is a group derived from cholesterol.

In one embodiment, the anionic compound bearing carboxylate charges and hydrophobic radicals according to the invention is chosen from the compounds of formula II in which the -Hy group is a group derived from a tocopherol.

In one embodiment, the anionic compound bearing carboxylate charges and hydrophobic radicals according to the invention is chosen from the compounds of formula II in which the -Hy group is a group derived from a tocopherol derivative, chosen from the racemate, the L isomer or the D isomer of α-tocopherol.

In one embodiment, the anionic compound bearing carboxylate charges and hydrophobic radicals according to the invention is chosen from the compounds of formula II in which the -Hy group is a group derived from DL-α-tocopherol.

In one embodiment, the anionic compound bearing carboxylate charges and hydrophobic radicals, according to the invention, is chosen from the compounds of formula II in which the -Hy group is an alkyl group derived from a hydrophobic acid.

In one embodiment, the anionic compound bearing carboxylate charges and hydrophobic radicals according to the invention is chosen from the compounds of formula II in which the -Hy group is an alkyl group derived from a fatty acid.

In one embodiment, the anionic compound bearing carboxylate charges and hydrophobic radicals according to the invention is chosen from the compounds of formula II in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the anionic compound bearing carboxylate charges and hydrophobic radicals according to the invention is chosen from the compounds of formula II in which the -Hy group is an alkyl group derived from a fatty acid chosen from the group consisting of linear fatty acids.

In one embodiment, the anionic compound bearing carboxylate charges and hydrophobic radicals according to the invention is chosen from the compounds of formula II in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the anionic compound bearing carboxylate charges and hydrophobic radicals according to the invention is chosen from the compounds of formula II in which the -Hy group is an alkyl group derived from an unsaturated fatty acid.

In one embodiment, the anionic compound bearing carboxylate charges and hydrophobic radicals according to the invention is chosen from the compounds of formula II in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the anionic compound bearing carboxylate charges and hydrophobic radicals according to the invention is chosen from the compounds of formula II in which the -Hy group is an alkyl group derived from a bile acid and derivatives thereof.

In one embodiment, the anionic compound bearing carboxylate charges and hydrophobic radicals according to the invention is chosen from the compounds of formula II in which the -Hy group is an alkyl group derived from a bile acid and derivatives thereof, chosen from the group consisting of cholic acid, dehydrocholic acid, deoxycholic acid and chenodeoxycholic acid.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from cholesterol.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from α-tocopherol.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from cholesterol, f is a carbamate function, and -f-[A]-COOH is derived from glycine.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from cholesterol, f is a carbamate function, and -f-[A]-COOH is derived from aspartic acid.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from cholesterol, f is a carbamate function, and -f-[A]-COOH is derived from glutamic acid.

In one embodiment, the anionic compound bearing carboxylate charges and hydrophobic radicals according to the invention is chosen from the compounds of formula II in which the radical —R″ is a radical -k-[Hy], such that k is a carbamate function and the -Hy group is derived from α-tocopherol, f is a carbamate function, and -f-[A]-COOH is derived from glycine.

In one embodiment, in the anionic compound bearing carboxylate charges and hydrophobic radicals according to the invention the backbone before substitution with radicals bearing carboxylate charges and hydrophobic radicals is TRIS.

In one embodiment, the composition according to the invention is characterized in that the anionic compound bearing carboxylate charges and hydrophobic radicals is chosen from the compounds of formula I in which a=0, b=1, —X— is a radical —C═O—, —R″ is a hydrogen atom and —R₁ is a radical —NH-[E]-(o-[Hy])ₜ.

In one embodiment, the composition according to the invention is characterized in that the anionic compound bearing carboxylate charges and hydrophobic radicals is a compound chosen from the compounds of formula I in which a=0, b=1, —X— is a radical —C═O—, —R″ is a hydrogen atom and —R₁ is a radical —NH-[E]-(o-[Hy])ₜ of formula III:

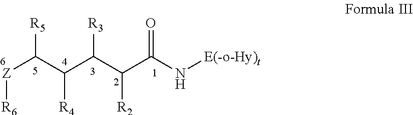

in which:
1) —R₂, —R₃, —R₄ and —R₅, which may be identical or different, are radicals -f-[A]-COOH, and
2) —R₆ is chosen from the group consisting of:
   —OH;
   -f-[A]-COOH if —Z—═CH₂;
   —O-[D] or —NH-[D] if —Z—═—C═O—;
3) -A-, -D-, -E- -Hy, t, f and o are as defined above,
4) f and o being identical or different,
5) the free acid functions being in the form of salts of alkali metal cations chosen from the group consisting of Na⁺ and K⁺.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from an amino acid.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from an alpha amino acid.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a beta amino acid.

In one embodiment, the composition according to the invention is characterized in that the anionic compound bearing carboxylate charges and hydrophobic radicals is chosen from the compounds of formula III in which the at least divalent radical —NH-E-(o-)ₜ, derived from a beta amino acid, is β-alanine.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a mono- or polyethylene glycol amine.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a mono- or polyethylene glycol diamine.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical derived from ethylenediamine.

In one embodiment, the composition according to the invention is characterized in that the anionic compound bearing carboxylate charges and hydrophobic radicals is chosen from the compounds of formula III in which the function o is an ester function.

In one embodiment, the composition according to the invention is characterized in that the anionic compound bearing carboxylate charges and hydrophobic radicals is chosen from the compounds of formula III in which the function o is an amide function.

In one embodiment, the composition according to the invention is characterized in that the anionic compound bearing carboxylate charges and hydrophobic radicals is chosen from the compounds of formula III in which the function o is a carbamate function.

In one embodiment, the composition according to the invention is characterized in that the anionic compound bearing carboxylate charges and hydrophobic radicals is chosen from the compounds of formula III in which the function o is a carbonate function.

In one embodiment, the composition according to the invention is characterized in that the anionic compound bearing carboxylate charges and hydrophobic radicals is chosen from the compounds of formula III in which the -Hy group is an alkyl group derived from a branched or unbranched, unsaturated and/or saturated, hydrophobic alcohol comprising from 12 to 30 carbons.

In one embodiment, the composition according to the invention is characterized in that the anionic compound bearing carboxylate charges and hydrophobic radicals is chosen from the compounds of formula III in which the -Hy group is an alkyl group derived from a hydrophobic alcohol chosen from the group consisting of dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound bearing carboxylate charges and hydrophobic radicals is chosen from the compounds of formula III in which the -Hy group is a group derived from a sterol.

In one embodiment, the composition according to the invention is characterized in that the anionic compound bearing carboxylate charges and hydrophobic radicals is chosen from the compounds of formula III in which the -Hy group is a group derived from a sterol, chosen from the group consisting of cholesterol and derivatives thereof.

In one embodiment, the composition according to the invention is characterized in that the anionic compound bearing carboxylate charges and hydrophobic radicals is chosen from the compounds of formula III in which the -Hy group is a group derived from cholesterol.

In one embodiment, the composition according to the invention is characterized in that the anionic compound bearing carboxylate charges and hydrophobic radicals is chosen from the compounds of formula III in which the -Hy group is a group derived from a tocopherol.

In one embodiment, the composition according to the invention is characterized in that the anionic compound bearing carboxylate charges and hydrophobic radicals is chosen from the compounds of formula III in which the -Hy group is a group derived from a tocopherol derivative, chosen from the racemate, the L isomer or the D isomer of α-tocopherol.

In one embodiment, the composition according to the invention is characterized in that the anionic compound bearing carboxylate charges and hydrophobic radicals is chosen from the compounds of formula III in which the -Hy group is derived from DL-α-tocopherol.

In one embodiment, the composition according to the invention is characterized in that the anionic compound bearing carboxylate charges and hydrophobic radicals is chosen from the compounds of formula III in which the radical -o-[Hy] is such that o is a carbamate function and the -Hy group is derived from cholesterol.

In one embodiment, the composition according to the invention is characterized in that the anionic compound bearing carboxylate charges and hydrophobic radicals is chosen from the compounds of formula III in which the radical -o-[Hy] is such that o is a carbamate function and the -Hy group is derived from α-tocopherol.

In one embodiment, the composition according to the invention is characterized in that the anionic compound bearing carboxylate charges and hydrophobic radicals is chosen from the compounds of formula III in which the -Hy group is an alkyl group derived from a hydrophobic acid.

In one embodiment, the composition according to the invention is characterized in that the anionic compound bearing carboxylate charges and hydrophobic radicals is chosen from the compounds of formula III in which the -Hy group is an alkyl group derived from a fatty acid.

In one embodiment, the composition according to the invention is characterized in that the anionic compound bearing carboxylate charges and hydrophobic radicals is chosen from the compounds of formula III in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the composition according to the invention is characterized in that the anionic compound bearing carboxylate charges and hydrophobic radicals is chosen from the compounds of formula III in which the -Hy group is an alkyl group derived from a fatty acid chosen from the group consisting of linear fatty acids.

In one embodiment, the composition according to the invention is characterized in that the anionic compound bearing carboxylate charges and hydrophobic radicals is chosen from the compounds of formula III in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound bearing carboxylate charges and hydrophobic radicals is chosen from the compounds of formula III in which the -Hy group is an alkyl group derived from an unsaturated fatty acid.

In one embodiment, the composition according to the invention is characterized in that the anionic compound bearing carboxylate charges and hydrophobic radicals is chosen from the compounds of formula III in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound bearing carboxylate charges and hydrophobic radicals is chosen from the compounds of formula III in which the -Hy group is an alkyl group derived from a bile acid and derivatives thereof.

In one embodiment, the composition according to the invention is characterized in that the anionic compound bearing carboxylate charges and hydrophobic radicals is chosen from the compounds of formula III in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound bearing carboxylate charges and hydrophobic radicals is chosen from the compounds of formula III in which the -Hy group is derived from cholesterol, the radical —NH-E-(o-)ₜ is derived from ethylenediamine, o and f are carbamate functions, and the radical -f-[A]-COOH is derived from aspartic acid.

In one embodiment, the composition according to the invention is characterized in that the anionic compound bearing carboxylate charges and hydrophobic radicals is chosen from the compounds of formula III in which the -Hy group is derived from racemic α-tocopherol, the radical —NH-E-(o-)ₜ is derived from ethylenediamine, o and f are carbamate functions, and the radical -f-[A]-COOH is derived from aspartic acid.

In one embodiment, the composition according to the invention is characterized in that the backbone before substitution with radicals bearing carboxylate charges and hydrophobic radicals is gluconolactone.

In one embodiment, the composition according to the invention is characterized in that the backbone before substitution with radicals bearing carboxylate charges and hydrophobic radicals is mucic acid.

In one embodiment, the composition according to the invention is characterized in that the anionic compound bearing carboxylate charges and hydrophobic radicals is a compound chosen from the compounds of formula III in which Z═CH₂, of formula VIII:

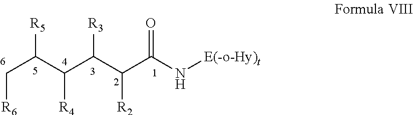

1) —R₂, —R₃, —R₄, —R₅ and —R₆, which may be identical or different, are radicals -f-[A]-COOH,
2) -f-[A]-COOH, comprising from 2 to 6 carbon atoms, is derived from an amino acid,
3) -D-, -E-, -Hy, t and o are as defined above,
4) f is a carbamate function,
5) f and o being identical or different,
6) the free acid functions being in the form of salts of alkali metal cations chosen from the group consisting of Na⁺ and K⁺.

In one embodiment, the composition according to the invention is characterized in that the anionic compound bearing carboxylate charges and hydrophobic radicals is chosen from the compounds of formula VIII in which the radical -f-[A]-COOH is derived from an alpha amino acid.

In one embodiment, the composition according to the invention is characterized in that the anionic compound bearing carboxylate charges and hydrophobic radicals is chosen from the compounds of formula VIII in which the radical -f-[A]-COOH is derived from glycine.

In one embodiment, the composition according to the invention is characterized in that the anionic compound bearing carboxylate charges and hydrophobic radicals is chosen from the compounds of formula VIII in which the radical -f-[A]-COOH is derived from aspartic acid.

In one embodiment, the composition according to the invention is characterized in that the anionic compound bearing carboxylate charges and hydrophobic radicals is chosen from the compounds of formula VIII in which the radical -f-[A]-COOH is derived from glutamic acid.

In one embodiment, the composition according to the invention is characterized in that the anionic compound bearing carboxylate charges and hydrophobic radicals is chosen from the compounds of formula VIII in which the radical -f-[A]-COOH is derived from a beta amino acid.

In one embodiment, the composition according to the invention is characterized in that the anionic compound bearing carboxylate charges and hydrophobic radicals is chosen from the compounds of formula VIII in which the radical -f-[A]-COOH is derived from β-alanine.

In one embodiment, the composition according to the invention is characterized in that the anionic compound bearing carboxylate charges and hydrophobic radicals is chosen from the compounds of formula VIII in which the function f is an ether function.

In one embodiment, the composition according to the invention is characterized in that the anionic compound bearing carboxylate charges and hydrophobic radicals is chosen from the compounds of formula VIII in which the function f is a carbamate function.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from an amino acid.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from an alpha amino acid.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a beta amino acid.

In one embodiment, the composition according to the invention is characterized in that the anionic compound bearing carboxylate charges and hydrophobic radicals is chosen from the compounds of formula VIII in which the at least divalent radical —NH-E-(o-)ₜ, derived from a beta amino acid, is β-alanine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol amine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol diamine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from ethylenediamine.

In one embodiment, the composition according to the invention is characterized in that the anionic compound bearing carboxylate charges and hydrophobic radicals is chosen from the compounds of formula VIII in which the function o is an ester function.

In one embodiment, the composition according to the invention is characterized in that the anionic compound bearing carboxylate charges and hydrophobic radicals is chosen from the compounds of formula VIII in which the function o is an amide function.

In one embodiment, the composition according to the invention is characterized in that the anionic compound bearing carboxylate charges and hydrophobic radicals is chosen from the compounds of formula VIII in which the function o is a carbamate function.

In one embodiment, the composition according to the invention is characterized in that the anionic compound bearing carboxylate charges and hydrophobic radicals is chosen from the compounds of formula VIII in which the function o is a carbonate function.

In one embodiment, the composition according to the invention is characterized in that the anionic compound bearing carboxylate charges and hydrophobic radicals is chosen from the compounds of formula VIII in which the -Hy group is an alkyl group derived from a branched or unbranched, unsaturated and/or saturated, hydrophobic alcohol comprising from 12 to 30 carbons.

In one embodiment, the composition according to the invention is characterized in that the anionic compound bearing carboxylate charges and hydrophobic radicals is chosen from the compounds of formula VIII in which the -Hy group is an alkyl group derived from a hydrophobic alcohol chosen from the group consisting of dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound bearing carboxylate charges and hydrophobic radicals is chosen from the compounds of formula VIII in which the -Hy group is a group derived from a sterol.

In one embodiment, the composition according to the invention is characterized in that the anionic compound bearing carboxylate charges and hydrophobic radicals is chosen from the compounds of formula VIII in which the -Hy group is a group derived from a sterol, chosen from the group consisting of cholesterol and derivatives thereof.

In one embodiment, the composition according to the invention is characterized in that the anionic compound bearing carboxylate charges and hydrophobic radicals is chosen from the compounds of formula VIII in which the -Hy group is a group derived from cholesterol.

In one embodiment, the composition according to the invention is characterized in that the anionic compound bearing carboxylate charges and hydrophobic radicals is chosen from the compounds of formula VIII in which the -Hy group is a group derived from a tocopherol.

In one embodiment, the composition according to the invention is characterized in that the anionic compound bearing carboxylate charges and hydrophobic radicals is chosen from the compounds of formula VIII in which the -Hy group is a group derived from a tocopherol derivative, chosen from the racemate, the L isomer or the D isomer of α-tocopherol.

In one embodiment, the composition according to the invention is characterized in that the anionic compound bearing carboxylate charges and hydrophobic radicals is chosen from the compounds of formula VIII in which the -Hy group is derived from DL-α-tocopherol.

In one embodiment, the composition according to the invention is characterized in that the anionic compound bearing carboxylate charges and hydrophobic radicals is chosen from the compounds of formula VIII in which the radical -o-[Hy] is such that o is a carbamate function and the -Hy group is derived from cholesterol.

In one embodiment, the composition according to the invention is characterized in that the anionic compound bearing carboxylate charges and hydrophobic radicals is chosen from the compounds of formula VIII in which the radical -o-[Hy] is such that o is a carbamate function and the -Hy group is derived from α-tocopherol.

In one embodiment, the composition according to the invention is characterized in that the anionic compound bearing carboxylate charges and hydrophobic radicals is chosen from the compounds of formula VIII in which the -Hy group is an alkyl group derived from a hydrophobic acid.

In one embodiment, the composition according to the invention is characterized in that the anionic compound hearing carboxylate charges and hydrophobic radicals is chosen from the compounds of formula VIII in which the -Hy group is an alkyl group derived from a fatty acid.

In one embodiment, the composition according to the invention is characterized in that the anionic compound bearing carboxylate charges and hydrophobic radicals is chosen from the compounds of formula VIII in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the composition according to the invention is characterized in that the anionic compound bearing carboxylate charges and hydrophobic radicals is chosen from the compounds of formula VIII in which the -Hy group is an alkyl group derived from a fatty acid chosen from the group consisting of linear fatty acids,

In one embodiment, the composition according to the invention is characterized in that the anionic compound bearing carboxylate charges and hydrophobic radicals is chosen from the compounds of formula VIII in which the -Hy group is an alkyl group derived horn a saturated linear fatty acid chosen from the group consisting of lauric (dodecanoic) acid, myristic (tetradecanoic) acid, palmitic (hexadecanoic) acid, stearic (octadecanoic) acid, acid, arachidic (eicosanoic) acid, behenic (docosenoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound bearing carboxylate charges and hydrophobic radicals is chosen from the compounds of formula VIII in which the -Hy group is an alkyl group derived from an unsaturated fatty acid.

In one embodiment, the composition according to the invention is characterized in that the anionic compound bearing carboxylate charges and hydrophobic radicals is chosen from the compounds of formula VIII in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound bearing carboxylate charges and hydrophobic radicals is chosen from the compounds of formula VIII in which the -Hy group is an alkyl group derived from a bile acid and derivatives thereof.

In one embodiment, the composition according to the invention is characterized in that the anionic compound bearing carboxylate charges and hydrophobic radicals is chosen from the compounds of formula VIII in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound bearing carboxylate charges and hydrophobic radicals is chosen from the compounds of formula VIII in which the -Hy group is derived from cholesterol, the radical —NH-E-(o-)ₜ is derived from ethylenediamine, o and fare carbamate functions, and the radical -f-[A]-COOH is derived from aspartic acid.

In one embodiment, the composition according to the invention is characterized in that the anionic compound bearing carboxylate charges and hydrophobic radicals is chosen from the compounds of formula VIII in which the -Hy group is derived from racemic α-tocopherol, the radical —NH-E-(o-)ₜ is derived from ethylenediamine, o and f are carbamate functions, and the radical -f-[A]-COOH is derived from aspartic acid.

In one embodiment, the composition according to the invention is characterized in that the backbone before substitution with radicals bearing carboxylate charges and hydrophobic radicals is gluconolactone.

In one embodiment, the composition according to the invention is characterized in that the backbone before substitution with radicals bearing carboxylate charges and hydrophobic radicals is mucic acid.

The invention also relates to an anionic compound bearing carboxylate charges and hydrophobic radicals chosen from the compounds of formula I, in which a=0, b=1, —X— is a radical —C═O—, —R″ is a hydrogen atom and —R₁ is a radical —NH-[E]-(o-[Hy])ₜ:

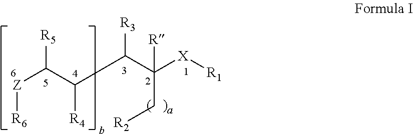

in which:
1) a=0, and
2) b=1, and
3) —X— is a radical —C═O—; and
4) —Z— is either a radical —C═O—, or a radical —CH₂—; and
5) —R₂, —R₃, —R₄ and —R₅ are chosen from the group consisting of the radicals:
   i. —OH;
   ii. -f-[A]-COOH;
   in which,
      -A- is an at least divalent radical comprising from 1 to 15 carbon atoms comprising at least one heteroatom chosen from O, N and S, optionally bearing carboxyl or amine functions and/or -f-[A]-COOH, comprising from 2 to 16 carbon atoms, is derived from an amino acid, from a diacid or from an alcohol acid and is bonded to the backbone of the molecule via a function f;
      f is chosen from the group consisting of ether, ester, carbamate, amide or carbonate functions;
6) —R″ is a hydrogen atom,
7) —R₁ is a radical —NH-[E]-(o-[Hy])ₜ,
   i. -E- is an at least divalent radical comprising from 1 to 15 carbon atoms comprising at least one heteroatom chosen from O, N and S optionally bearing carboxyl or amine functions and/or NH-[E]-(o-)ₜ, comprising from 2 to 16 carbon atoms, is derived from an amino acid from a diamine or from an amine alcohol;
   ii. -Hy is a C₁₂ to C₃₀ linear or cyclic alkyl group or a C₁₂ to C₃₀ alkylaryl or arylalkyl, optionally substituted with one or more to C₁ to C₂ alkyl groups, which is derived from a hydrophobic compound;
   iii. o is an ester, amide, carbamate or carbonate function; and
   iv. t be a positive integer equal to 1 or 2;
8) —R₆ is chosen from the group consisting of the radicals;
   i. —OH
   ii. -f-[A]-COOH if —Z—═—CH₂—; or
   iii. —O-[D] if —Z—═—C═O—;
   iv. —NH-[D] if —Z—═—C═O—;
   v. the radical -D being -[E]-(o-[Hy])ₜ
      -A-, f, o and Hy being defined as above;
      and
9) the asymmetrical carbon atoms are of absolute configuration R or S; the free acid functions being in the form of salts of alkali metal cations chosen from the group consisting of Na⁺ and K⁺.

In one embodiment, the anionic compound bearing carboxylate charges and hydrophobic radicals is chosen from the compounds of formula I in which the radical -f-[A]-COOH is chosen from the radicals of formula VI below:

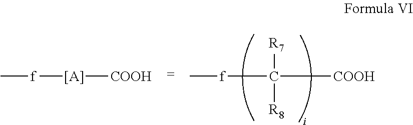

in which:
i is greater than or equal to 1 and less than or equal to 12, and
R₇ and R₈, which may be identical or different, are chosen from the group consisting of a hydrogen atom, a saturated or unsaturated, linear, branched or cyclic C₁ to C₆ alkyl, a benzyl, an alkylaryl, optionally comprising heteroatoms chosen from the group consisting of O, N and/or S, or functions chosen from the group consisting of carboxylic acid, amine, alcohol and thiol functions,
-f-[A]-COOH comprises from 2 to 16 carbon atoms.

In one embodiment, the anionic compound bearing carboxylate charges and hydrophobic radicals is chosen from the compounds of formula I in which the radical -f-[A]-COOH, comprising from 2 to 8 carbon atoms, is derived from an amino acid, from a dialcohol, from a diamine, from a diacid or from an amine alcohol.

In one embodiment, the anionic compound bearing carboxylate charges and hydrophobic radicals is chosen from the compounds of formula I in which the radical -f-[A]-COOH, comprising from 2 to 6 carbon atoms, is derived from an amino acid, from a dialcohol, from a diamine, from a diacid or from an amine alcohol.

In one embodiment, the anionic compound bearing carboxylate charges and hydrophobic radicals is chosen from the compounds of formula I in which the radical -f-[A]-COOH is chosen from the group consisting of the following sequences, f having the meaning given above:

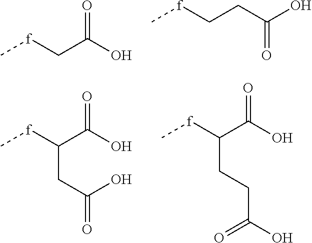

or the salts thereof with alkali metal cations chosen from the group consisting of Na⁺ and K⁺.

In one embodiment, the anionic compound bearing carboxylate charges and hydrophobic radicals is chosen from the compounds of formula I in which the radical -f-[A]-COOH is -f-CH₂—COOH.

In one embodiment, the anionic compound bearing carboxylate charges and hydrophobic radicals is chosen from the compounds of formula I in which the radical -f-[A]-COOH is derived from an alpha amino acid.

In one embodiment, the anionic compound bearing carboxylate charges and hydrophobic radicals is chosen from the compounds of formula I in which the radical -f-[A]-COOH is derived from glycine.

In one embodiment, the anionic compound bearing carboxylate charges and hydrophobic radicals is chosen from the compounds of formula I in which the radical -f-[A]-COOH is derived from succinic acid.

In one embodiment, the anionic compound bearing carboxylate charges and hydrophobic radicals is chosen from the compounds of formula I in which the radical -f-[A]-COOH is derived from aspartic acid.

In one embodiment, the anionic compound bearing carboxylate charges and hydrophobic radicals is chosen from the compounds of formula I in which the radical -f-[A]-COOH is derived from glutamic acid.

In one embodiment, the anionic compound bearing carboxylate charges and hydrophobic radicals is chosen from the compounds of formula I in which the radical -f-[A]-COOH is derived from a beta amino acid.

In one embodiment, the anionic compound bearing carboxylate charges and hydrophobic radicals is chosen from the compounds of formula I in which the radical -f-[A]-COOH is derived from O-alanine.

In one embodiment, the anionic compound bearing carboxylate charges and hydrophobic radicals is chosen from the compounds of formula I in which the function f is an ether function.

In one embodiment, the anionic compound bearing carboxylate charges and hydrophobic radicals is chosen from the compounds of formula I in which the function f is a carbamate function.

In one embodiment, the anionic compound bearing carboxylate charges and hydrophobic radicals is chosen from the compounds of formula I in which the function f is an ester function.

In one embodiment, the anionic compound bearing carboxylate charges and hydrophobic radicals is chosen from the compounds of formula I in which the function f is a carbonate function.

In one embodiment, the anionic compound bearing carboxylate charges and hydrophobic radicals is chosen from the compounds of formula I in which the function f is an amide function.

In one embodiment, the anionic compound bearing carboxylate charges and hydrophobic radicals according to the invention is a compound chosen from the compounds of formula I in which a=0, b=1, —X— is a radical —C═O—, —R″ is a hydrogen atom and —R₁ is a radical —NH-[E]-(o-[Hy])ₜ of formula III:

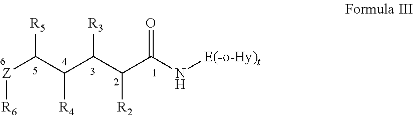

in which:
1) —R₂, —R₃, —R₄ and —R₅, which may be identical or different, are radicals -f-[A]-COOH, and
2) —R₆ is chosen from the group consisting of:
   —OH;
   -f-[A]-COOH if —Z—═CH₂;
   —O-[D] or —NH-[D] if —Z—═—C═O—;
3) -A-, -D-, -E- -Hy, t, f and o are as defined above,
4) f and o being identical or different,
5) the free acid functions being in the form of salts of alkali metal cations chosen from the group consisting of Na⁺ and K⁺.

In one embodiment, the composition according to the invention is characterized in that the anionic compound bearing carboxylate charges and hydrophobic radicals is chosen from the compounds of formula III in which the radical —NH-E-(o-)ₜ is an at least divalent radical, comprising from 2 to 9 carbon atoms, is derived from an amino acid, from a dialcohol, from a diamine, from a diacid or from an amine alcohol.

In one embodiment, the anionic compound bearing carboxylate charges and hydrophobic radicals according to the invention is chosen from the compounds of formula III in which the radical —NH-E-(o-)ₜ is an at least divalent radical derived from an amino acid.

In one embodiment, the anionic compound bearing carboxylate charges and hydrophobic radicals according to the invention is chosen from the compounds of formula III in which the radical —NH-E-(o-)ₜ is an at least divalent radical derived from an alpha amino acid.

In one embodiment, the anionic compound bearing carboxylate charges and hydrophobic radicals according to the invention is chosen from the compounds of formula III in which the radical —NH-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the anionic compound bearing carboxylate charges and hydrophobic radicals according to the invention is chosen from the compounds of formula III in which the radical —NH-E-(o-)ₜ is an at least divalent radical derived from a beta amino acid.

In one embodiment, the anionic compound bearing carboxylate charges and hydrophobic radicals according to the invention is chosen from the compounds of formula III in which the at least divalent radical —NH-E-(o-)ₜ, derived from a beta amino acid, is β-alanine.

In one embodiment, the anionic compound bearing carboxylate charges and hydrophobic radicals according to the invention is chosen from the compounds of formula III in which the radical —NH-E-(o-)ₜ is an at least divalent radical derived from a mono- or polyethylene glycol amine.

In one embodiment, the anionic compound bearing carboxylate charges and hydrophobic radicals according to the invention is chosen from the compounds of formula III in which the radical —NH-E-(o-)ₜ is an at least divalent radical derived from a mono- or polyethylene glycol amine chosen from the group consisting of ethanolamine, diethylene glycol amine and triethylene glycol amine.

In one embodiment, the anionic compound bearing carboxylate charges and hydrophobic radicals according to the invention is chosen from the compounds of formula III in which the radical —NH-E-(o-)ₜ is an at least divalent radical derived from a mono- or polyethylene glycol diamine.

In one embodiment, the anionic compound bearing carboxylate charges and hydrophobic radicals according to the invention is chosen from the compounds of formula III in which the radical —NH-E-(o-)ₜ is an at least divalent radical derived from a mono- or polyethylene glycol diamine chosen from the group consisting of diethylene glycol diamine and triethylene glycol diamine.

In one embodiment, the anionic compound bearing carboxylate charges and hydrophobic radicals according to the invention is chosen from the compounds of formula III in which the radical —NH-E-(o-)ₜ is an at least divalent radical derived from ethylenediamine.

In one embodiment, the anionic compound bearing carboxylate charges and hydrophobic radicals according to the invention is chosen from the compounds of formula III in which the function o is an ester function.

In one embodiment, the anionic compound bearing carboxylate charges and hydrophobic radicals according to the invention is chosen from the compounds of formula III in which the function o is an amide function.

In one embodiment, the anionic compound bearing carboxylate charges and hydrophobic radicals according to the invention is chosen from the compounds of formula III in which the function o is a carbamate function.

In one embodiment, the anionic compound bearing carboxylate charges and hydrophobic radicals according to the invention is chosen from the compounds of formula III in which the function o is a carbonate function.

In one embodiment, the anionic compound bearing carboxylate charges and hydrophobic radicals according to the invention is chosen from the compounds of formula III in which the -Hy group is an alkyl group derived from a branched or unbranched, unsaturated and/or saturated, hydrophobic alcohol comprising from 12 to 30 carbons.

In one embodiment, the anionic compound bearing carboxylate charges and hydrophobic radicals according to the invention is chosen from the compounds of formula III in which the -Hy group is an alkyl group derived from a hydrophobic alcohol chosen from the group consisting of dodecanol (lauryl alcohol), tetradecanol

(myristyl alcohol), hexadecanol (cetyl alcohol), octadecanol (stearyl alcohol), cetearyl alcohol and oleyl alcohol.

In one embodiment, the anionic compound bearing carboxylate charges and hydrophobic radicals according to the invention is chosen from the compounds of formula III in which the -Hy group is a group derived from a sterol.

In one embodiment, the anionic compound bearing carboxylate charges and hydrophobic radicals according to the invention is chosen from the compounds of formula III in which the -Hy group is a group derived from a sterol, chosen from the group consisting of cholesterol and derivatives thereof.

In one embodiment, the anionic compound bearing carboxylate charges and hydrophobic radicals according to the invention is chosen from the compounds of formula III in which the -Hy group is a group derived from cholesterol.

In one embodiment, the anionic compound bearing carboxylate charges and hydrophobic radicals according to the invention is chosen from the compounds of formula III in which the -Hy group is a group derived from a tocopherol.

In one embodiment, the anionic compound bearing carboxylate charges and hydrophobic radicals according to the invention is chosen from the compounds of formula III in which the -Hy group is a group derived from a tocopherol derivative, chosen from the racemate, the L isomer or the D isomer of α-tocopherol.

In one embodiment, the anionic compound bearing carboxylate charges and hydrophobic radicals according to the invention is chosen from the compounds of formula III in which the -Hy group is derived from DL-α-tocopherol.

In one embodiment, the anionic compound bearing carboxylate charges and hydrophobic radicals according to the invention is chosen from the compounds of formula III in which the radical -o-[Hy] is such that o is a carbamate function and the -Hy group is derived from cholesterol.

In one embodiment, the anionic compound bearing carboxylate charges and hydrophobic radicals according to the invention is chosen from the compounds of formula III in which the radical -o-[Hy] is such that o is a carbamate function and the -Hy group is derived from α-tocopherol.

In one embodiment, the anionic compound bearing carboxylate charges and hydrophobic radicals according to the invention is chosen from the compounds of formula III in which the -Hy group is an alkyl group derived from a hydrophobic acid.

In one embodiment, the anionic compound bearing carboxylate charges and hydrophobic radicals according to the invention is chosen from the compounds of formula III in which the -Hy group is an alkyl group derived from a fatty acid.

In one embodiment, the anionic compound bearing carboxylate charges and hydrophobic radicals according to the invention is chosen from the compounds of formula III in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the anionic compound bearing carboxylate charges and hydrophobic radicals according to the invention is chosen from the compounds of formula III in which the -Hy group is an alkyl group derived from a fatty acid chosen from the group consisting of linear fatty acids.

In one embodiment, the anionic compound bearing carboxylate charges and hydrophobic radicals according to the invention is chosen from the compounds of formula III in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the anionic compound bearing carboxylate charges and hydrophobic radicals according to the invention is chosen from the compounds of formula III in which the -Hy group is an alkyl group derived from an unsaturated fatty acid.

In one embodiment, the anionic compound bearing carboxylate charges and hydrophobic radicals according to the invention is chosen from the compounds of formula III in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the anionic compound bearing carboxylate charges and hydrophobic radicals according to the invention is chosen from the compounds of formula III in which the -Hy group is an alkyl group derived from a bile acid and derivatives thereof.

In one embodiment, the anionic compound bearing carboxylate charges and hydrophobic radicals according to the invention is chosen from the compounds of formula III in which the -Hy group is an alkyl group derived from a bile acid and derivatives thereof, chosen from the group consisting of cholic acid, dehydrocholic acid, deoxycholic acid and chenodeoxycholic acid.

In one embodiment, the anionic compound bearing carboxylate charges and hydrophobic radicals according to the invention is chosen from the compounds of formula III in which the -Hy group is derived from cholesterol, the radical —NH-E-(o-)ₜ is derived from ethylenediamine, o and f are carbamate functions, and the radical -f-[A]-COOH is derived from aspartic acid.

In one embodiment, the anionic compound bearing carboxylate charges and hydrophobic radicals according to the invention is chosen from the compounds of formula III in which the -Hy group is derived from racemic α-tocopherol, the radical —NH-E-(o-)ₜ is derived from ethylenediamine, o and f are carbamate functions, and the radical -f-[A]-COOH is derived from aspartic acid.

In one embodiment, in the anionic compound bearing carboxylate charges and hydrophobic radicals according to the invention the backbone before substitution with radicals bearing carboxylate charges and hydrophobic radicals is gluconolactone.

In one embodiment, in the anionic compound bearing carboxylate charges and hydrophobic radicals according to the invention the backbone before substitution with radicals bearing carboxylate charges and hydrophobic radicals is mucic acid.

In one embodiment, the anionic compound bearing carboxylate charges and hydrophobic radicals according to the invention is a compound chosen from the compounds of formula III in which Z═CH₂, of formula VIII:

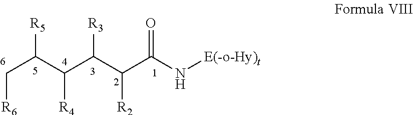

1) —R₂, —R₃, —R₄, —R₅ and —R₆, which may be identical or different, are radicals -f-[A]-COOH,
2) -f-[A]-COOH, comprising from 2 to 6 carbon atoms, is derived from an amino acid,
3) -D-, -E-, -Hy, t and o are as defined above,
4) f is a carbamate function,
5) f and o being identical or different,
6) the free acid functions being in the form of salts of alkali metal cations chosen from the group consisting of Na⁺ and K⁺.

In one embodiment, the composition according to the invention is characterized in that the anionic compound bearing carboxylate charges and hydrophobic radicals is chosen from the compounds of formula VIII in which the radical -f-[A]-COOH is derived from an alpha amino acid.

In one embodiment, the composition according to the invention is characterized in that the anionic compound bearing carboxylate charges and hydrophobic radicals is chosen from the compounds of formula VIII in which the radical -f-[A]-COOH is derived from glycine.

In one embodiment, the composition according to the invention is characterized in that the anionic compound bearing carboxylate charges and hydrophobic radicals is chosen from the compounds of formula VIII in which the radical -f-[A]-COOH is derived from aspartic acid.

In one embodiment, the composition according to the invention is characterized in that the anionic compound bearing carboxylate charges and hydrophobic radicals is chosen from the compounds of formula VIII in which the radical -f-[A]-COOH is derived from glutamic acid.

In one embodiment, the composition according to the invention is characterized in that the anionic compound bearing carboxylate charges and hydrophobic radicals is chosen from the compounds of formula VIII in which the radical -f-[A]-COOH is derived from a beta amino acid.

In one embodiment, the composition according to the invention is characterized in that the anionic compound bearing carboxylate charges and hydrophobic radicals is chosen from the compounds of formula VIII in which the radical -f-[A]-COOH is derived from β-alanine.

In one embodiment, the composition according to the invention is characterized in that the anionic compound bearing carboxylate charges and hydrophobic radicals is chosen from the compounds of formula VIII in which the function f is an ether function.

In one embodiment, the composition according to the invention is characterized in that the anionic compound bearing carboxylate charges and hydrophobic radicals is chosen from the compounds of formula VIII in which the function f is a carbamate function.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from an amino acid.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from an alpha amino acid.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a beta amino acid.

In one embodiment, the composition according to the invention is characterized in that the anionic compound bearing carboxylate charges and hydrophobic radicals is chosen from the compounds of formula VIII in which the at least divalent radical —NH-E-(o-)ₜ derived from a beta amino acid, is β-alanine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol amine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol diamine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound bearing carboxylate charges and hydrophobic radicals is chosen from the compounds of formula VIII in which the radical —NH-E-(o-)ₜ is an at least divalent radical derived from ethylenediamine.

In one embodiment, the composition according to the invention is characterized in that the anionic compound bearing carboxylate charges and hydrophobic radicals is chosen from the compounds of formula VIII in which the function o is an ester function.

In one embodiment, the composition according to the invention is characterized in that the anionic compound bearing carboxylate charges and hydrophobic radicals is chosen from the compounds of formula VIII in which the function o is an amide function.

In one embodiment, the composition according to the invention is characterized in that the anionic compound bearing carboxylate charges and hydrophobic radicals is chosen from the compounds of formula VIII in which the function o is a carbamate function.

In one embodiment, the composition according to the invention is characterized in that the anionic compound bearing carboxylate charges and hydrophobic radicals is chosen from the compounds of formula VIII in which the function o is a carbonate function.

In one embodiment, the composition according to the invention is characterized in that the anionic compound bearing carboxylate charges and hydrophobic radicals is chosen from the compounds of formula VIII in which the -Hy group is an alkyl group derived from a branched or unbranched, saturated and/or unsaturated, hydrophobic alcohol comprising from 12 to 30 carbons.

In one embodiment, the composition according to the invention is characterized in that the anionic compound bearing carboxylate charges and hydrophobic radicals is chosen from the compounds of formula VIII in which the -Hy group is an alkyl group derived from a hydrophobic alcohol chosen from the group consisting of dodecanol (lauryl alcohol), tetradecanol (myristyl alcohol), hexadecanol (cetyl alcohol), octadecanol (stearyl alcohol cetearyl alcohol and oleyl alcohol.

In one embodiment, the composition according to the invention is characterized in that the anionic compound bearing carboxylate charges and hydrophobic radicals is chosen from the compounds of formula VIII in which the -Hy group is a group derived from a sterol.

In one embodiment, the composition according to the invention is characterized in that the anionic compound bearing carboxylate charges and hydrophobic radicals is chosen from the compounds of formula VIII in which the -Hy group is a group derived from a sterol, chosen from the group consisting of cholesterol and derivatives thereof.

In one embodiment, the composition according to the invention is characterized in that the anionic compound bearing carboxylate charges and hydrophobic radicals is chosen from the compounds of formula VIII in which the -Hy group is a group derived from cholesterol.

In one embodiment, the composition according to the invention is characterized in that the anionic compound bearing carboxylate charges and hydrophobic radicals is chosen from the compounds of formula VIII in which the -Hy group is a group derived from a tocopherol.

In one embodiment, the composition according to the invention is characterized in that the anionic compound bearing carboxylate charges and hydrophobic radicals is chosen from the compounds of formula VIII in which the -Hy group is a group derived from a tocopherol derivative, chosen from the racemate, the L isomer or the D isomer of α-tocopherol.

In one embodiment, the composition according to the invention is characterized in that the anionic compound bearing carboxylate charges and hydrophobic radicals is chosen from the compounds of formula VIII in which the -Hy group is derived from DL-α-tocopherol.

In one embodiment, the composition according to the invention is characterized in that the anionic compound bearing carboxylate charges and hydrophobic radicals is chosen from the compounds of formula VIII in which the radical -o-[Hy] is such that o is a carbamate function and the -Hy group is derived from cholesterol.

In one embodiment, the composition according to the invention is characterized in that the anionic compound bearing carboxylate charges and hydrophobic radicals is chosen from the compounds of formula VIII in which the radical -o-[Hy] is such that o is a carbamate function and the -Hy group is derived from α-tocopherol.

In one embodiment, the composition according to the invention is characterized in that the anionic compound bearing carboxylate charges and hydrophobic radicals is chosen from the compounds of formula VIII in which the -Hy group is an alkyl group derived from a hydrophobic acid.

In one embodiment, the composition according to the invention is characterized in that the anionic compound bearing carboxylate charges and hydrophobic radicals is chosen from the compounds of formula VIII in which the -Hy group is an alkyl group derived from a fatty acid.

In one embodiment, the composition according to the invention is characterized in that the anionic compound bearing carboxylate charges and hydrophobic radicals is chosen from the compounds of formula VIII in which the -Hy group is an alkyl group derived from a fatty acid chosen from the group consisting of the acids consisting of a branched or unbranched, unsaturated or saturated, alkyl chain comprising from 12 to 30 carbon atoms.

In one embodiment, the composition according to the invention is characterized in that the anionic compound bearing carboxylate charges and hydrophobic radicals is chosen from the compounds of formula VIII in which the -Hy group is an alkyl group derived from a fatty acid chosen from the group consisting of linear fatty acids.

In one embodiment, the composition according to the invention is characterized in that the anionic compound bearing carboxylate charges and hydrophobic radicals is chosen from the compounds of formula VIII in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound bearing carboxylate charges and hydrophobic radicals is chosen from the compounds of formula VIII in which the -Hy group is an alkyl group derived from an unsaturated fatty acid.

In one embodiment, the composition according to the invention is characterized in that the anionic compound bearing carboxylate charges and hydrophobic radicals is chosen from the compounds of formula VIII in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound bearing carboxylate charges and hydrophobic radicals is chosen from the compounds of formula VIII in which the -Hy group is an alkyl group derived from a bile acid and derivatives thereof.

In one embodiment, the composition according to the invention is characterized in that the anionic compound bearing carboxylate charges and hydrophobic radicals is chosen from the compounds of formula VIII in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound bearing carboxylate charges and hydrophobic radicals is chosen from the compounds of formula VIII in which the -Hy group is derived from cholesterol, the radical —NH-E-(o-)ₜ is derived from ethylenediamine, o and f are carbamate functions, and the radical -f-[A]-COOH is derived from aspartic acid.

In one embodiment, the composition according to the invention is characterized in that the anionic compound bearing carboxylate charges and hydrophobic radicals is chosen from the compounds of formula VIII in which the -Hy group is derived from racemic α-tocopherol, the radical —NH-E-(o-)ₜ is derived from ethylenediamine, o and f are carbamate functions, and the radical -f-[A]-COOH is derived from aspartic acid.

In one embodiment, the composition according to the invention is characterized in that the backbone before substitution with radicals bearing carboxylate charges and hydrophobic radicals is gluconolactone.

In one embodiment, the composition according to the invention is characterized in that the backbone before substitution with radicals bearing carboxylate charges and hydrophobic radicals is mucic acid.

In one embodiment, the composition according to the invention is characterized in that the anionic compound bearing carboxylate charges and hydrophobic radicals is chosen from the compounds of formula I in which a=0, b=1, —X— is a radical —CH₂— and —R″ is a hydrogen atom.

In one embodiment, the composition according to the invention is characterized in that the anionic compound bearing carboxylate charges and hydrophobic radicals of formula I, in which a=0, b=1, —X— is a radical —CH₂— and —R″ is a hydrogen atom, is a compound chosen from the compounds of formula IV:

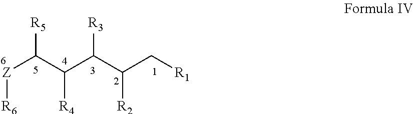

in which:
1) —R₆ is chosen from the group consisting of the radicals —OH, -f-[A]-COOH and -g-[B]-(k-[D])ₚ,
2) —R₁ is chosen from the group consisting of the radicals —OH, -f-[A]-COOH and -g-[B]-(k-[D])ₚ,
3) at most one of —R₂, —R₃, —R₄ and —R₆ is a backbone formed from a discrete number u of between 1 and 7 (1≦u≦7) of identical or different saccharide units linked via identical or different glycosidic linkages, at least one saccharide unit being chosen from the group consisting of:
   i. hexoses in cyclic form or in open reduced form,
   ii. uronic acids in cyclic form or in open oxidized form,
   iii. hexosamines in cyclic form, in open reduced form or in open oxidized form,
   iv. N-acetylhexosamines in cyclic form or in open reduced form,
at least one of said saccharide units being substituted with at least one substituent —R′=-f-[A]-COOH, and/or at least one substituent —R′, which may be identical or different, chosen from the group consisting of -k-[D] and -g-[B]-(k-[D])ₚ;
-A-, —B—, -D-, f, g and k being defined as above;
and/or
—R₃ and —R₄, which may be identical or different, are chosen from the group consisting of the radicals:
   i. —OH; or
   ii. -f-[A]-COOH; or
   iii. -g-[B]-(k-[D])ₚ;
-A-, —B—, -D-, f, g and k being defined as above;
and/or
—R₂ is chosen from the group consisting of the radicals:
   i. —OH; or
   ii. -f-[A]-COOH; or
   iii. -g-[B]-(k-[D])ₚ; or
   iv. —NH—COCH₃; or
   v. —NH₂;
   vi. —NH-[D];
-A-, —B—, -D-, f, g and k being defined as above;
and/or
—R₆ is chosen from the group consisting of the radicals:
   i. —OH; or
   ii. -f-[A]-COOH if —Z—═—CH₂—; or
   iii. -g-[B]-(k-[D])ₚ if —Z—═—CH₂—; or
   iv. —O-[D], if —Z—═—C═O—;
   v. —NH-[D], if —Z—═—C═O—;
-A-, —B—, -D-, f, g and k being defined as above;
4) —X—, —Z—, -A-, —B—, -D-, -E-, -Hy, t, p, f, g, k and o are defined as above,
5) f, g, k and o being identical or different;
6) the free acid functions being in the form of salts of alkali metal cations chosen from the group consisting of Na⁺ and K⁺;
7) the degree of substitution with carboxylate charges per saccharide unit is greater than or equal to 0.4;
8) the degree of substitution with hydrophobic radicals per saccharide unit is less than or equal to 0.5.

In one embodiment, the composition according to the invention is characterized in that the anionic compound bearing carboxylate charges and hydrophobic radicals is chosen from the compounds of formula IV in which —Z—═—C═O—.

In one embodiment, the composition according to the invention is characterized in that the anionic compound bearing carboxylate charges and hydrophobic radicals is chosen from the compounds of formula IV in which —Z—═—CH₂—.

In one embodiment, the anionic compound bearing carboxylate charges and hydrophobic radicals, of formula IV, is in the isolated state or as a mixture.

In one embodiment, the composition according to the invention is characterized in that the anionic compound bearing carboxylate charges and hydrophobic radicals is chosen from the compounds of formula IV in which the radical -g-[B]— is chosen from the radicals of formula VII below:

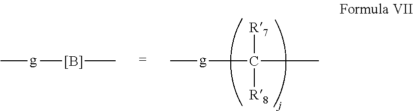

in which:
j is greater than or equal to 1 and less than or equal to 12, and
—R′₇ and —R′₈, which may be identical or different, are chosen from the group consisting of a hydrogen atom, a saturated or unsaturated, linear, branched or cyclic C₁ to C₆ alkyl, a benzyl, an alkylaryl, optionally comprising heteroatoms chosen from the group consisting of O, N and/or S, or functions chosen from the group consisting of carboxylic acid, amine, alcohol and thiol functions,
-g-[B]— comprises from 1 to 15 carbon atoms.

In one embodiment, the composition according to the invention is characterized in that the anionic compound bearing carboxylate charges and hydrophobic radicals is chosen from the compounds of formula IV in which the radical -g-[B]-(k-)ₚ, comprising from 2 to 8 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula IV in which the radical -g-[B]-(k-)ₚ, comprising from 2 to 6 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula IV in which the radical -g-[B]-(k-[D])ₚ is chosen from the group consisting of the following sequences; g, k and D having the meanings given above:

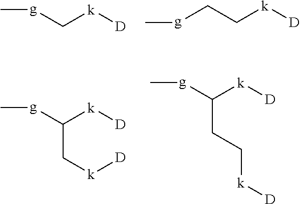

In one embodiment, the composition according to the invention is characterized in that the anionic compound bearing carboxylate charges and hydrophobic radicals is chosen from the compounds of formula IV in which the radical -g-[B]-(k-[D])ₚ is such that —B— is —CH₂—.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from an alpha amino acid.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from glycine.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from succinic acid.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from aspartic acid.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from glutamic acid.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from a beta amino acid.

In one embodiment, the composition according to the invention is characterized in that the anionic compound bearing carboxylate charges and hydrophobic radicals is chosen from the compounds of formula IV in which the radical -g-[B]-(k-[D])ₚ is such that -g-[B]-(k-)ₚ is derived from β-alanine.

In one embodiment, the composition according to the invention is characterized in that the anionic compound bearing carboxylate charges and hydrophobic radicals is chosen from the compounds of formula IV in which the function g is an ether function.

In one embodiment, the composition according to the invention is characterized in that the anionic compound bearing carboxylate charges and hydrophobic radicals is chosen from the compounds of formula IV in which the function g is a carbamate function.

In one embodiment, the composition according to the invention is characterized in that the anionic compound bearing carboxylate charges and hydrophobic radicals is chosen from the compounds of formula IV in which the function g is an ester function.

In one embodiment, the composition according to the invention is characterized in that the anionic compound bearing carboxylate charges and hydrophobic radicals is chosen from the compounds of formula IV in which the function g is a carbonate function.

In one embodiment, the composition according to the invention is characterized in that the anionic compound bearing carboxylate charges and hydrophobic radicals is chosen from the compounds of formula IV in which the function g is an amide function.

In one embodiment, the composition according to the invention is characterized in that the anionic compound bearing carboxylate charges and hydrophobic radicals is chosen from the compounds of formula IV in which the function k is an amide function.

In one embodiment, the composition according to the invention is characterized in that the anionic compound bearing carboxylate charges and hydrophobic radicals is chosen from the compounds of formula IV in which the function k is a carbamate function.

In one embodiment, the composition according to the invention is characterized in that the anionic compound bearing carboxylate charges and hydrophobic radicals is chosen from the compounds of formula IV in which the function k is an ester function.

In one embodiment, the composition according to the invention is characterized in that the anionic compound bearing carboxylate charges and hydrophobic radicals is chosen from the compounds of formula IV in which the function k is a carbonate function.

In one embodiment, the composition according to the invention is characterized in that the anionic compound bearing carboxylate charges and hydrophobic radicals is chosen from the compounds of formula IV in which the radical -g-[B]-(k-[D])ₚ is such that -D is the radical -[E]-(o-Hy)ₜ, and p=1, k, o, t, -E- and -Hy having the definitions given above.

In one embodiment, the composition according to the invention is characterized in that the anionic compound bearing carboxylate charges and hydrophobic radicals is chosen from the compounds of formula IV in which the radical -k-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula IV in which the radical -k-E-(o-)ₜ is an at least divalent radical derived from an amino acid.

In one embodiment, the composition according to the invention is characterized in that the anionic compound bearing carboxylate charges and hydrophobic radicals is chosen from the compounds of formula IV in which the radical -k-E-(o-)ₜ is an at least divalent radical derived from an alpha amino acid.

In one embodiment, the composition according to the invention is characterized in that the anionic compound is chosen from the compounds of formula IV in which the radical -k-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula IV in which the radical -k-E-(o-)ₜ is an at least divalent radical derived from a beta amino acid.

In one embodiment, the composition according to the invention is characterized in that the anionic compound is chosen from the compounds of formula IV in which the at least divalent radical -k-E-(o-)ₜ, derived from a beta amino acid, is β-alanine.

In one embodiment, the composition according to the invention is characterized in that the anionic compound is chosen from the compounds of formula IV in which the radical -k-E-(o-)ₜ is an at least divalent radical derived from a mono- or polyethylene glycol.

In one embodiment, the composition according to the invention is characterized in that the anionic compound is chosen from the compounds of formula IV in which the radical -k-E-(o-)ₜ is an at least divalent radical derived from ethylene glycol.

In one embodiment, the composition according to the invention is characterized in that the anionic compound is chosen from the compounds of formula IV in which the radical -k-E-(o-)ₜ is an at least divalent radical derived from a polyethylene glycol chosen from the group consisting of diethylene glycol, triethylene glycol and tetraethylene glycol.

In one embodiment, the composition according to the invention is characterized in that the anionic compound is chosen from the compounds of formula IV in which the radical -k-E-(o-)ₜ is an at least divalent radical derived from a mono- or polyethylene glycol amine.

In one embodiment, the composition according to the invention is characterized in that the anionic compound is chosen from the compounds of formula IV in which the radical -k-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound is chosen from the compounds of formula IV in which the radical -k-E-(o-)ₜ is an at least divalent radical derived from a mono- or polyethylene glycol diamine.

In one embodiment, the composition according to the invention is characterized in that the anionic compound is chosen from the compounds of formula IV in which the radical -k-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound is chosen from the compounds of formula IV in which the radical -k-E-(o-)ₜ is an at least divalent radical derived from ethylenediamine.

In one embodiment, the composition according to the invention is characterized in that the anionic compound is chosen from the compounds of formula IV in which the function o is an ester function.

In one embodiment, the composition according to the invention is characterized in that the anionic compound is chosen from the compounds of formula IV in which the function o is an amide function.

In one embodiment, the composition according to the invention is characterized in that the anionic compound is chosen from the compounds of formula IV in which the function o is a carbamate function.

In one embodiment, the composition according to the invention is characterized in that the anionic compound is chosen from the compounds of formula IV in which the function o is a carbonate function.

In one embodiment, the composition according to the invention is characterized in that the anionic compound bearing carboxylate charges and hydrophobic radicals is chosen from the compounds of formula IV in which the radical -g-[B]-(k-[D])ₚ is such that -D is the radical -Hy, and p=1 or 2, -Hy having the definition given above.

In one embodiment, the composition according to the invention is characterized in that the anionic compound is chosen from the compounds of formula IV in which the -Hy group is an alkyl group derived from a branched or unbranched, unsaturated and/or saturated, hydrophobic alcohol comprising from 8 to 30 carbons.

In one embodiment, the composition according to the invention is characterized in that the anionic compound is chosen from the compounds of formula IV in which the -Hy group is an alkyl group derived from a hydrophobic alcohol chosen from the group consisting of octanol, decanol, 3,7-dimethyl-1-octyl, dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound is chosen from the compounds of formula IV in which the -Hy group is a group derived from a sterol.

In one embodiment, the composition according to the invention is characterized in that the anionic compound is chosen from the compounds of formula IV in which the -Hy group is a group derived from a sterol, chosen from the group consisting of cholesterol and derivatives thereof.

In one embodiment, the composition according to the invention is characterized in that the anionic compound is chosen from the compounds of formula IV in which the -Hy group is a group derived from cholesterol.

In one embodiment, the composition according to the invention is characterized in that the anionic compound is chosen from the compounds of formula IV in which the -Hy group is a group derived from a tocopherol.

In one embodiment, the composition according to the invention is characterized in that the anionic compound is chosen from the compounds of formula IV in which the -Hy group is a group derived from a tocopherol derivative, chosen from the racemate, the L isomer or the D isomer of α-tocopherol.

In one embodiment, the composition according to the invention is characterized in that the anionic compound is chosen from the compounds of formula IV in which the -Hy group is a group derived from DL-α-tocopherol.

In one embodiment, the composition according to the invention is characterized in that the anionic compound is chosen from the compounds of formula IV in which the -Hy group is a group derived from a menthol derivative.

In one embodiment, the composition according to the invention is characterized in that the anionic compound is chosen from the compounds of formula IV in which the -Hy group is a group derived from a menthol derivative, chosen from the racemate, the L isomer or the D isomer of menthol.

In one embodiment, the composition according to the invention is characterized in that the anionic compound is chosen from the compounds of formula IV in which the -Hy group is a group derived from an alcohol bearing an aryl group.

In one embodiment, the composition according to the invention is characterized in that the anionic compound is chosen from the compounds of formula IV in which the -Hy group is a group derived from phenethyl alcohol.

In one embodiment, the composition according to the invention is characterized in that the anionic compound is chosen from the compounds of formula IV in which the -Hy group is an alkyl group derived from a hydrophobic acid.

In one embodiment, the composition according to the invention is characterized in that the anionic compound is chosen from the compounds of formula IV in which the -Hy group is an alkyl group derived from a fatty acid.

In one embodiment, the composition according to the invention is characterized in that the anionic compound is chosen from the compounds of formula IV in which the -Hy group is an alkyl group derived from a fatty acid chosen from the group consisting of the acids consisting of a branched or unbranched, unsaturated or saturated, alkyl chain comprising from 8 to 30 carbons.

In one embodiment, the composition according to the invention is characterized in that the anionic compound is chosen from the compounds of formula IV in which the -Hy group is an alkyl group derived from a fatty acid chosen from the group consisting of linear fatty acids.

In one embodiment, the composition according to the invention is characterized in that the anionic compound is chosen from the compounds of formula IV in which the -Hy group is an alkyl group derived from a saturated linear fatty acid chosen from the group consisting of caprylic (octanoic) acid, nonanoic acid, capric (decanoic) acid, undecylic (undecanoic) acid, lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid

In one embodiment, the composition according to the invention is characterized in that the anionic compound is chosen from the compounds of formula IV in which the -Hy group is an alkyl group derived from an unsaturated fatty acid.

In one embodiment, the composition according to the invention is characterized in that the anionic compound is chosen from the compounds of formula IV in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound is chosen from the compounds of formula IV in which the -Hy group is an alkyl group derived from a bile acid and derivatives thereof.

In one embodiment, the composition according to the invention is characterized in that the anionic compound is chosen from the compounds of formula IV in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound is chosen from the compounds of formula IV in which the -D group is -[E]-(o-[Hy])ₜ in which t=1, -k-E-(o-)ₜ is derived from leucine, o is an ester function, and -Hy is derived from cholesterol.

In one embodiment, the composition according to the invention is characterized in that the anionic compound is chosen from the compounds of formula IV in which the radical -g-[B]-(k-[D])ₚ is such that g is an ether function, —B— is a radical —CH₂—, p=1, k is an amide function, -D is the radical -[E]-(o-[Hy])ₜ, t=1, -k-E-(o-)ₜ is derived from leucine, o is an ester function and -Hy is derived from cholesterol.

In one embodiment, the composition according to the invention is characterized in that the anionic compound bearing carboxylate charges and hydrophobic radicals is chosen from the compounds of formula IV in which at most one of —R₂, —R₃, —R₄ and —R₆ is a backbone formed from a discrete number u of between 1 and 7 (1≦u≦7) of identical or different saccharide units, substituted with at least one substituent R′.

In one embodiment, the composition according to the invention is characterized in that the anionic compound bearing carboxylate charges and hydrophobic radicals is such that the at least one substituent R′ is a radical -f-[A]-COOH, -k-[D] and/or a radical -g-[B]-k-[D])ₚ, -A-, —B—, -D-, f, g, k and p being defined as above.

In one embodiment, the composition according to the invention is characterized in that the anionic compound bearing carboxylate charges and hydrophobic radicals is chosen from the compounds of formula IV in which the identical or different saccharide units are chosen from the group consisting of hexoses, uronic acids, hexosamines and N-acylhexosamines.

In one embodiment, the composition according to the invention is characterized in that the identical or different hexoses are in cyclic form or in open reduced form.

In one embodiment, the composition according to the invention is characterized in that the hexoses are identical or different cyclic hexoses and are chosen from the group consisting of fructose, sorbose, tagatose, psicose, glucose, mannose, galactose, allose, altrose, talose, idose, gulose, fucose, fuculose and rhamnose.

In one embodiment, the composition according to the invention is characterized in that the hexoses are identical or different, open reduced hexoses chosen from the group consisting of mannitol, xylitol, sorbitol and galactitol (dulcitol).

In one embodiment, the composition according to the invention is characterized in that the identical or different uronic acids are in cyclic form or in open oxidized form.

In one embodiment, the composition according to the invention is characterized in that the identical or different uronic acids in cyclic form are chosen from the group consisting of glucuronic acid, iduronic acid and galacturonic acid.

In one embodiment, the composition according to the invention is characterized in that the identical or different uronic acids in open oxidized form are chosen from the group consisting of gluconic acid, glucaric acid and galactonic acid.

In one embodiment, the composition according to the invention is characterized in that the identical or different hexosamines are in cyclic form, in open reduced form or in open oxidized form.

In one embodiment, the composition according to the invention is characterized in that the identical or different hexosamines in cyclic form are chosen from the group consisting of glucosamine, galactosamine and mannosamine.

In one embodiment, the composition according to the invention is characterized in that the hexosamine in open reduced form is meglumine.

In one embodiment, the composition according to the invention is characterized in that the hexosamine in open oxidized form is glucosaminic acid.

In one embodiment, the composition according to the invention is characterized in that the identical or different N-acetylhexosamines are in cyclic form or in open reduced form.

In one embodiment, the composition according to the invention is characterized in that the identical or different N-acetylhexosamines in cyclic form are chosen from the group consisting of N-acetylglucosamine, N-acetylgalactosamine and N-acetylmannosamine.

In one embodiment, the composition according to the invention is characterized in that the anionic compound bearing carboxylate charges and hydrophobic radicals is chosen from the compounds of formula IV in which u=7.

In one embodiment, the composition according to the invention is characterized in that the anionic compound bearing carboxylate charges and hydrophobic radicals is chosen from the compounds of formula IV in which u=6.

In one embodiment, the composition according to the invention is characterized in that the anionic compound bearing carboxylate charges and hydrophobic radicals is chosen from the compounds of formula IV in which u=5.

In one embodiment, the composition according to the invention is characterized in that the anionic compound bearing carboxylate charges and hydrophobic radicals is chosen from the compounds of formula IV in which u=4.

In one embodiment, the composition according to the invention is characterized in that the anionic compound bearing carboxylate charges and hydrophobic radicals is chosen from the compounds of formula IV in which u=3.

In one embodiment, the composition according to the invention is characterized in that the anionic compound bearing carboxylate charges and hydrophobic radicals is chosen from the compounds of formula IV in which u=2.

In one embodiment, the composition according to the invention is characterized in that the anionic compound bearing carboxylate charges and hydrophobic radicals is chosen from the compounds of formula IV in which u=1.

In one embodiment, the composition according to the invention is characterized in that the anionic compound bearing carboxylic charges and hydrophobic radicals is chosen from the compounds of formula IV in which the glucosidic linkages of the radical formed from a discrete number u of saccharide units of the anionic compound are of 1,2, 1,3, 1, 4 or 1,6 type, which may be identical or different.

In one embodiment, the composition according to the invention is characterized in that the anionic compound bearing carboxylic charges and hydrophobic radicals is chosen from the compounds of formula IV in which the glucosidic linkages of the radical formed from a discrete number u of saccharide units of the anionic compound are of α and/or β type, which may be identical or different.

In one embodiment, the composition according to the invention is characterized in that the anionic compound bearing carboxylate charges and hydrophobic radicals is chosen from the compounds of formula IV in which the radical —R₄ is a saccharide sequence such that u=2 and the glucosidic linkages are of 1,4 type.

In one embodiment, the composition according to the invention is characterized in that the anionic compound bearing carboxylate charges and hydrophobic radicals is chosen from the compounds of formula IV in which the radical —R₄ is a saccharide sequence such that u=3 and the glucosidic linkages are of 1,4 type.

In one embodiment, the composition according to the invention is characterized in that the anionic compound bearing carboxylate charges and hydrophobic radicals is chosen from the compounds of formula IV in which the radical —R₄ is a saccharide sequence such that u=4 and the glucosidic linkages are of 1,4 type.

In one embodiment, the composition according to the invention is characterized in that the anionic compound bearing carboxylate charges and hydrophobic radicals is chosen from the compounds of formula IV in which the radical —R₄ is a saccharide sequence such that u=5 and the glucosidic linkages are of 1,4 type.

In one embodiment, the composition according to the invention is characterized in that the anionic compound bearing carboxylate charges and hydrophobic radicals is chosen from the compounds of formula IV in which the radical —R₄ is a saccharide sequence such that u=6 and the glucosidic linkages are of 1,4 type.

In one embodiment, the composition according to the invention is characterized in that the anionic compound bearing carboxylate charges and hydrophobic radicals is chosen from the compounds of formula IV in which the radical —R₄ is a saccharide sequence such that u=7 and the glucosidic linkages are of 1,4 type.

In one embodiment, the composition according to the invention is characterized in that the anionic compound bearing carboxylate charges and hydrophobic radicals is chosen from the compounds of formula IV in which —Z— is the radical —CH₂—, f is an ether bond, -A- is the radical CH₂, g is an ether function, —B— is the radical —CH₂—, p=1, k is an amide function, -k-E-(o-)ₜ is derived from leucine, o is an ester function, -Hy is derived from cholesterol, the radical —R₄ is a saccharide sequence such that u=2 and the glucosicidic linkages are of 1,4 type.

In one embodiment, the composition according to the invention is characterized in that the anionic compound bearing carboxylate charges and hydrophobic radicals is chosen from the compounds of formula IV in which —Z— is the radical —CH₂—, f is an ether bond, -A- is the radical —CH₂—, g is an ether function, —B— is the radical —CH₂—, p=1, k is an amide function, -k-E-(o-)ₜ is derived from leucine, o is an ester function, -Hy is derived from cholesterol, the radical —R₄ is a saccharide sequence such that u=4 and the saccharide linkages are of 1,4 type.

In one embodiment, the composition according to the invention is characterized in that the anionic compound bearing carboxylate charges and hydrophobic radicals is chosen from the compounds of formula IV in which —Z— is the radical —CH₂—, f is an ether bond, -A- is the radical CH₂, g is an ether function, —B— is the radical —CH₂—, p=1, k is an amide function, -k-E-(o-)ₜ is derived from leucine, o is an ester function, -Hy is derived from cholesterol, the radical —R₄ is a saccharide sequence such that u=7 and the saccharide linkages are of 1,4 type.

In one embodiment, the composition according to the invention is characterized in that the backbone before substitution with radicals bearing carboxylate charges and hydrophobic radicals is composed of 3 saccharide units, the glucosidic linkages of which are of a 1,4 type, and is maltotriose.

In one embodiment, the composition according to the invention is characterized in that the backbone before substitution with radicals bearing carboxylate charges and hydrophobic radicals is composed of 5 saccharide units, the glucosidic linkages of which are of a 1,4 type, and is maltopentaose.

In one embodiment, the composition according to the invention is characterized in that the backbone before substitution with radicals bearing carboxylate charges and hydrophobic radicals is composed of 8 saccharide units, the glucosidic linkages of which are of a 1,4 type, and is maltooctaose.

In one embodiment, the composition according to the invention is characterized in that the anionic compound bearing carboxylate charges and hydrophobic radicals of formula IV, in which Z═CH₂, p=1 and t=1, is a compound chosen from the compounds of formula IX:

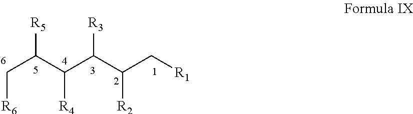

in which:
1) R₁, R₂, R₃, R₄, R₅ and R₆ are defined as above,
2) -A-, —B—, f, g and k are defined as above,
3) D is a radical -[E]-o-[Hy],
4) -E- and o are defined as above,
5) -Hy is a C₂₀ to C₃₀ linear or cyclic alkyl group or a C₂₀ to C₃₀ alkylaryl or arylalkyl, optionally substituted with one or more C₁ to C₃ alkyl groups, which is derived from a hydrophobic compound,
6) f, g, k and o being identical or different,
7) the free acid functions being in the form of salts of alkali metal cations chosen from the group consisting of Na⁺ and K⁺,
8) the degree of substitution with carboxylate charges per saccharide unit is greater than or equal to 0.4,
9) the degree of substitution with hydrophobic radicals per saccharide unit is less than or equal to 0.5.

In one embodiment, the composition according to the invention is characterized in that the anionic compound is chosen from the compounds of formula IX in which the -Hy group is an alkyl group derived from a branched or unbranched, unsaturated and/or saturated, hydrophobic alcohol comprising from 20 to 30 carbons.

In one embodiment, the composition according to the invention is characterized in that the anionic compound is chosen from the compounds of formula IX in which the -Hy group is a group derived from a sterol.

In one embodiment, the composition according to the invention is characterized in that the anionic compound is chosen from the compounds of formula IX in which the -Hy group is a group derived from a sterol, chosen from the group consisting of cholesterol and derivatives thereof.

In one embodiment, the composition according to the invention is characterized in that the anionic compound is chosen from the compounds of formula IX in which the -Hy group is a group derived from cholesterol.

In one embodiment, the composition according to the invention is characterized in that the anionic compound is chosen from the compounds of formula IV in which the -Hy group is a group derived from a tocopherol.

In one embodiment, the composition according to the invention is characterized in that the anionic compound is chosen from the compounds of formula IX in which the -Hy group is a group derived from a tocopherol derivative, chosen from the racemate, the L isomer or the D isomer of α-tocopherol.

In one embodiment, the composition according to the invention is characterized in that the anionic compound is chosen from the compounds of formula IX in which the -Hy group is a group derived from DL-α-tocopherol.

In one embodiment, the composition according to the invention is characterized in that the anionic compound is chosen from the compounds of formula IX in which the -Hy group is an alkyl group derived from a hydrophobic acid.

In one embodiment, the composition according to the invention is characterized in that the anionic compound is chosen from the compounds of formula IX in which the -Hy group is an alkyl group derived from a fatty acid.

In one embodiment, the composition according to the invention is characterized in that the anionic compound is chosen from the compounds of formula IX in which the -Hy group is an alkyl group derived from a fatty acid chosen from the group consisting of the acids consisting of a branched or unbranched, unsaturated or saturated, alkyl chain comprising from 20 to 30 carbons.

In one embodiment, the composition according to the invention is characterized in that the anionic compound is chosen from the compounds of formula IX in which the -Hy group is an alkyl group derived from a fatty acid chosen from the group consisting of linear fatty acids.

In one embodiment, the composition according to the invention is characterized in that the anionic compound is chosen from the compounds of formula IX in which the -Hy group is an alkyl group derived from a saturated linear fatty acid chosen from the group consisting of arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound is chosen from the compounds of formula IX in which the -Hy group is an alkyl group derived from an unsaturated fatty acid.

In one embodiment, the composition according to the invention is characterized in that the anionic compound is chosen from the compounds of formula IX in which the -Hy group is an alkyl group derived from an unsaturated fatty acid chosen from the group consisting of arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound is chosen from the compounds of formula IX in which the -Hy group is an alkyl group derived from a bile acid and derivatives thereof.

In one embodiment, the composition according to the invention is characterized in that the anionic compound is chosen from the compounds of formula IX in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound is chosen from the compounds of formula IX in which the -D group is -[E]-(o-[Hy])ₜ in which t=1, -k-E-(o-)ₜ is derived from leucine, o is an ester function and -Hy is derived from cholesterol.

In one embodiment, the composition according to the invention is characterized in that the anionic compound is chosen from the compounds of formula IX in which the radical -g-[B]-(k-[D])ₚ is such that g is an ether function, —B— is a radical —CH₂—, p=1, k is an amide function, -D is the radical -[E]-(o[Hy])ₜ, t=1, -k-E-(o-)ₜ is derived from leucine, o is an ester function and -Hy is derived from cholesterol.

In one embodiment, the composition according to the invention is characterized in that the anionic compound bearing carboxylate charges and hydrophobic radicals of formula IV, in which Z═CH₂, p=1 and t=2, is a compound chosen from the compounds of formula X:

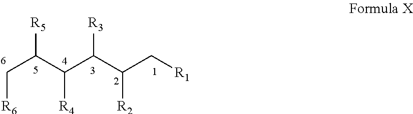

in which:
1) R₁, R₂, R₃, R₄, R₅ and R₆ are defined as above,
2) -A-, —B—, f, g and k are defined as above,
3) D is a radical -[E]-(o-[Hy])₂,
4) -k-E-(o-)₂, comprising 2 to 6 carbon atoms, are derived from a trivalent amino acid,
5) -Hy is a C₈ to C₂₀ linear or cyclic alkyl group or a C₆ to C₂₀ alkylaryl or arylalkyl, optionally substituted with one or more C₁ to C₃ alkyl groups, which is derived from a hydrophobic alcohol,
6) f, g and k being identical or different,
7) o is an ester function,
8) the free acid functions being in the form of salts of alkali metal cations chosen from the group consisting of Na⁺ and K⁺,
9) the degree of substitution with carboxylate charges per saccharide unit is greater than or equal to 0.4,
10) the degree of substitution with hydrophobic radicals per saccharide unit is less than or equal to 0.5.

In one embodiment, the composition according to the invention is characterized in that the anionic compound bearing carboxylate charges and hydrophobic radicals is chosen from the compounds of formula X in which the radical -k-E-(o-)₂ is an at least divalent radical derived from a trivalent alpha amino acid.

In one embodiment, the composition according to the invention is characterized in that the anionic compound is chosen from the compounds of formula X in which the radical -k-E-(o-)₂ is a trivalent radical derived from a natural alpha amino acid chosen from the group consisting of aspartic acid and glutamic acid, in their L, D or racemic forms.

In one embodiment, the composition according to the invention is characterized in that the anionic compound is chosen from the compounds of formula X in which the -Hy group is an alkyl group derived from a branched or unbranched, unsaturated and/or saturated, hydrophobic alcohol comprising from 8 to 20 carbons.

In one embodiment, the composition according to the invention is characterized in that the anionic compound is chosen from the compounds of formula X in which the -Hy group is an alkyl group derived from a hydrophobic alcohol chosen from the group consisting of octanol, decanol, 3,7-dimethyl-1-octyl, dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound is chosen from the compounds of formula X in which the -Hy group is an alkyl group derived from a branched or unbranched, unsaturated and/or saturated, hydrophobic alcohol comprising from 10 to 18 carbons.

In one embodiment, the composition according to the invention is characterized in that the anionic compound is chosen from the compounds of formula X in which the -Hy group is an alkyl group derived from a hydrophobic alcohol chosen from the group consisting of decanol, 3,7-dimethyl-1-octyl, dodecanol (lauryl alcohol), tetradecanol (myristyl alcohol), hexadecanol (cetyl alcohol) and octadecanol (stearyl alcohol).

In one embodiment, the composition according to the invention is characterized in that the anionic compound is chosen from the compounds of formula X in which the -Hy group is an alkyl group derived from a branched or unbranched, unsaturated and/or saturated, hydrophobic alcohol comprising from 12 to 18 carbons.

In one embodiment, the composition according to the invention is characterized in that the anionic compound is chosen from the compounds of formula X in which the -Hy group is an alkyl group derived from a hydrophobic alcohol chosen from the group consisting of dodecanol (lauryl alcohol), tetradecanol (myristyl alcohol), hexadecanol (cetyl alcohol) and octadecanol (stearyl alcohol).

In one embodiment, the composition according to the invention is characterized in that the anionic compound bearing carboxylate charges and hydrophobic radicals of formula IV, in which Z═CH₂, p=1 and t=1, is a compound chosen from the compounds of formula XI:

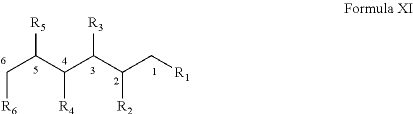

in which:
1) R₁, R₂, R₃, R₄, R₅ and R₆ are as defined above,
2) -A-, f, g and k are as defined above,
3) D is a radical -[E]-o-[Hy],
4) -k-E-o- are derived from a hydrophobic amino acid chosen from the group consisting of leucine, phenylalanine, isoleucine and valine, in their L, D or racemic forms,
5) -Hy is a C₆ to C₂₀ linear or cyclic alkyl group or a C₆ to C₂₀ alkylaryl or arylalkyl, optionally substituted with one or more C₁ to C₃ alkyl groups, which is derived from a hydrophobic alcohol,
6) f, g and k being identical or different,
7) o is an ester function,
8) the free acid functions being in the form of salts of alkali metal cations chosen from the group consisting of Na⁺ and K⁺,
9) the degree of substitution with carboxylate charges per saccharide unit is greater than or equal to 0.4,
10) the degree of substitution with hydrophobic radicals per saccharide unit is less than or equal to 0.5.

In one embodiment, the composition according to the invention is characterized in that the anionic compound is chosen from the compounds of formula XI in which the radical -k-E-(o-)₂ is a divalent radical derived from phenylalanine, in its L, D or racemic form.

In one embodiment, the composition according to the invention is characterized in that the anionic compound is chosen from the compounds of formula XI in which the -Hy group is an alkyl group derived from a branched or unbranched, unsaturated and/or saturated, hydrophobic alcohol comprising from 8 to 20 carbons.

In one embodiment, the composition according to the invention is characterized in that the anionic compound is chosen from the compounds of formula XI in which the -Hy group is an alkyl group derived from a hydrophobic alcohol chosen from the group consisting of octanol, decanol, 3,7-dimethyl-1-octyl, dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound is chosen from the compounds of formula XI in which the -Hy group is an alkyl group derived from a branched or unbranched, unsaturated and/or saturated, hydrophobic alcohol comprising from 10 to 18 carbons.

In one embodiment, the composition according to the invention is characterized in that the anionic compound is chosen from the compounds of formula XI in which the -Hy group is an alkyl group derived from a hydrophobic alcohol chosen from the group consisting of decanol, 3,7-dimethyl-1-octyl, dodecanol (lauryl alcohol), tetradecanol (myristyl alcohol), hexadecanol (cetyl alcohol) and octadecanol (stearyl alcohol).

In one embodiment, the composition according to the invention is characterized in that the anionic compound is chosen from the compounds of formula XI in which the -Hy group is an alkyl group derived from a branched or unbranched, unsaturated and/or saturated, hydrophobic alcohol comprising from 12 to 18 carbons.

In one embodiment, the composition according to the invention is characterized in that the anionic compound is chosen from the compounds of formula XI in which the -Hy group is an alkyl group derived from a hydrophobic alcohol chosen from the group consisting of dodecanol (lauryl alcohol), tetradecanol (myristyl alcohol), hexadecanol (cetyl alcohol) and octadecanol (stearyl alcohol).

In one embodiment, the composition according to the invention is characterized in that the anionic compound bearing carboxylate charges and hydrophobic radicals is chosen from the compounds of formula I in which a=0, b=1 and R″ is a hydrogen atom.

In one embodiment, the composition according to the invention is characterized in that the anionic compound bearing carboxylate charges and hydrophobic radicals is chosen from the compounds of formula I in which a=0, b=1 and R″ is a hydrogen atom, chosen from the compounds of formula V:

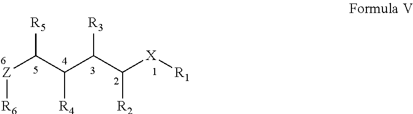

in which:
1) R₁ is either a radical —NH-[E]-(o-[Hy])ₜ, or a radical —N(L)_{z}-[E]-(o-[Hy])ₜ,
2) R₂, R₃, R₄ and R₆, which may be identical or different, are chosen from the group consisting of the radicals —OH and -f-[A]-COOH,
   and/or at most one of R₂, R₃, R₄ and R₅ is a backbone formed from a discrete number u of between 1 and 7 (1≦u≦7) of identical or different saccharide units substituted with at least one substituent R′=-f-[A]-COOH,
3) R₅, which may be identical or different, are either a radical —OH, or a radical -f-[A]-COOH,
4) -A-, —B—, -D-, L, -E-, -Hy, —X—, —Z—, t, f and o are defined as above,
5) the degree of substitution with carboxylate charges per saccharide unit is greater than or equal to 0.4,
6) f and o being identical or different,
7) the free acid functions being in the form of salts of alkali metal cations chosen from the group consisting of Na⁺ and K⁺.

In one embodiment, the anionic compound bearing carboxylate charges and hydrophobic radicals of formula V is in the isolated state or as a mixture.

In one embodiment, the composition according to the invention is characterized in that the anionic compound bearing carboxylate charges and hydrophobic radicals is chosen from the compounds of formula III in which —X—═—C═O—.

In one embodiment, the composition according to the invention is characterized in that the anionic compound bearing carboxylate charges and hydrophobic radicals is chosen from the compounds of formula V in which —X—═—CH₂—.

In one embodiment, the composition according to the invention is characterized in that the anionic compound bearing carboxylate charges and hydrophobic radicals is chosen from the compounds of formula V in which —Z—═—C═O—.

In one embodiment, the composition according to the invention is characterized in that the anionic compound bearing carboxylate charges and hydrophobic radicals is chosen from the compounds of formula V in which —Z—═—CH₂—.

In one embodiment, the composition according to the invention is characterized in that the anionic compound bearing carboxylate charges and hydrophobic radicals is chosen from the compounds of formula V in which R₁ is a radical —NH-[E]-(o-[Hy])ₜ.

In one embodiment, the composition according to the invention is characterized in that the anionic compound bearing carboxylate charges and hydrophobic radicals is chosen from the compounds of formula V in which R₁ is a radical —N(L)_{z}-[E]-(o-[Hy])ₜ.

In one embodiment, the composition according to the invention is characterized in that the anionic compound bearing carboxylate charges and hydrophobic radicals is chosen from the compounds of formula V in which the radical —NH-E-(o-)ₜ is an at least divalent radical, comprising from 2 to 9 carbon atoms, is derived from an amino acid, from a dialcohol, from a diamine, from a diacid or from an amine alcohol.

In one embodiment, the composition according to the invention is characterized in that the anionic compound bearing carboxylate charges and hydrophobic radicals is chosen from the compounds of formula V in which the radical —NH-E-(o-)ₜ is an at least divalent radical derived from an amino acid.

In one embodiment, the composition according to the invention is characterized in that the anionic compound bearing carboxylate charges and hydrophobic radicals is chosen from the compounds of formula V in which the radical —NH-E-(o-)ₜ is an at least divalent radical derived from an alpha amino acid.

In one embodiment, the composition according to the invention is characterized in that the anionic compound is chosen from the compounds of formula V in which the radical —NH-E-(o-)ₜ is an at least divalent radical derived from a natural alpha amino acid chosen from the group consisting of glycine, leucine, phenylalanine, lysine, isoleucine, alanine, valine, aspartic acid and glutamic acid, in their L, D or racemic forms.

In one embodiment, the composition according to the invention is characterized in that the anionic compound bearing carboxylate charges and hydrophobic radicals is chosen from the compounds of formula V in which the radical —NH-E-(o-)ₜ is an at least divalent radical derived from a beta amino acid.

In one embodiment, the composition according to the invention is characterized in that the anionic compound is chosen from the compounds of formula V in which the at least divalent radical —NH-E-(o-)ₜ, derived from a beta amino acid, is β-alanine.

In one embodiment, the composition according to the invention is characterized in that the anionic compound is chosen from the compounds of formula V in which the radical —X—═CH₂ and —NH-E-(o-)ₜ is an at least divalent radical derived from a mono- or polyethylene glycol.

In one embodiment, the composition according to the invention is characterized in that the anionic compound is chosen from the compounds of formula V in which the radical —X—═CH₂ and —NH-E-(o-)ₜ is an at least divalent radical derived from ethylene glycol.

In one embodiment, the composition according to the invention is characterized in that the anionic compound is chosen from the compounds of formula V in which the radical —X—═CH₂ and —NH-E-(o-)ₜ is an at least divalent radical derived from a polyethylene glycol chosen from the group consisting of diethylene glycol and triethylene glycol.

In one embodiment, the composition according to the invention is characterized in that the anionic compound is chosen from the compounds of formula V in which the radical —NH-E-(o-)ₜ is an at least divalent radical derived from a mono- or polyethylene glycol amine.

In one embodiment, the composition according to the invention is characterized in that the anionic compound is chosen from the compounds of formula V in which the radical —NH-E-(o-)ₜ is an at least divalent radical derived from a mono- or polyethylene glycol amine chosen from the group consisting of ethanolamine, diethylene glycol amine and triethylene glycol amine.

In one embodiment, the composition according to the invention is characterized in that the anionic compound is chosen from the compounds of formula V in which the radical —NH-E-(o-)ₜ is an at least divalent radical derived from a mono- or polyethylene glycol diamine.

In one embodiment, the composition according to the invention is characterized in that the anionic compound is chosen from the compounds of formula V in which the radical —NH-E-(o-)ₜ is an at least divalent radical derived from a mono- or polyethylene glycol diamine chosen from the group consisting of diethylene glycol diamine and triethylene glycol diamine.

In one embodiment, the composition according to the invention is characterized in that the anionic compound is chosen from the compounds of formula V in which the radical —NH-E-(o-)ₜ is an at least divalent radical derived from ethylenediamine.

In one embodiment, the composition according to the invention is characterized in that the anionic compound is chosen from the compounds of formula V in which the function o is an ester function.

In one embodiment, the composition according to the invention is characterized in that the anionic compound is chosen from the compounds of formula V in which the function o is an amide function.

In one embodiment, the composition according to the invention is characterized in that the anionic compound is chosen from the compounds of formula V in which the function o is a carbamate function.

In one embodiment, the composition according to the invention is characterized in that the anionic compound is chosen from the compounds of formula V in which the function o is a carbonate function.

In one embodiment, the composition according to the invention is characterized in that the anionic compound is chosen from the compounds of formula V in which the -Hy group is an alkyl group derived from a branched or unbranched, unsaturated and/or saturated, hydrophobic alcohol comprising from 8 to 30 carbons.

In one embodiment, the composition according to the invention is characterized in that the anionic compound is chosen from the compounds of formula V in which the -Hy group is an alkyl group derived from a hydrophobic alcohol chosen from the group consisting of octanol, decanol, 3,7-dimethyl-1-octyl, dodecanol (lauryl alcohol), tetradecanol (myristyl alcohol), hexadecanol (cetyl alcohol), octadecanol (stearyl alcohol), cetearyl alcohol and oleyl alcohol.

In one embodiment, the composition according to the invention is characterized in that the anionic compound is chosen from the compounds of formula V in which the -Hy group is a group derived from a menthol derivative.

In one embodiment, the composition according to the invention is characterized in that the anionic compound is chosen from the compounds of formula V in which the -Hy group is a group derived from a menthol derivative, chosen from the racemate, the L isomer or the D isomer of menthol.

In one embodiment, the composition according to the invention is characterized in that the anionic compound is chosen from the compounds of formula V in which the -Hy group is a group derived from an alcohol bearing an aryl group.

In one embodiment, the composition according to the invention is characterized in that the anionic compound is chosen from the compounds of formula V in which the -Hy group is a group derived from phenethyl alcohol.

In one embodiment, the composition according to the invention is characterized in that the anionic compound is chosen from the compounds of formula V in which the -Hy group is an alkyl group derived from a branched or unbranched, unsaturated and/or saturated, hydrophobic alcohol comprising from 12 to 30 carbons.

In one embodiment, the composition according to the invention is characterized in that the anionic compound is chosen from the compounds of formula V in which the -Hy group is a group derived from a sterol.

In one embodiment, the composition according to the invention is characterized in that the anionic compound is chosen from the compounds of formula V in which the -Hy group is a group derived from a sterol, chosen from the group consisting of cholesterol and derivatives thereof.

In one embodiment, the composition according to the invention is characterized in that the anionic compound is chosen from the compounds of formula V in which the -Hy group is a group derived from cholesterol.

In one embodiment, the composition according to the invention is characterized in that the anionic compound is chosen from the compounds of formula V in which the -Hy group is a group derived from a tocopherol.

In one embodiment, the composition according to the invention is characterized in that the anionic compound is chosen from the compounds of formula V in which the -Hy group is a group derived from a tocopherol derivative, chosen from the racemate, the L isomer or the D isomer of α-tocopherol.

In one embodiment, the composition according to the invention is characterized in that the anionic compound is chosen from the compounds of formula V in which the -Hy group is a group derived from DL-α-tocopherol.

In one embodiment, the composition according to the invention is characterized in that the anionic compound is chosen from the compounds of formula V in which the -Hy group is an alkyl group derived from a hydrophobic acid.

In one embodiment, the composition according to the invention is characterized in that the anionic compound is chosen from the compounds of formula V in which the -Hy group is an alkyl group derived from a fatty acid.

In one embodiment, the composition according to the invention is characterized in that the anionic compound is chosen from the compounds of formula V in which the -Hy group is an alkyl group derived from a fatty acid chosen from the group consisting of the acids consisting of a branched or unbranched, unsaturated or saturated, alkyl chain comprising from 12 to 30 carbons.

In one embodiment, the composition according to the invention is characterized in that the anionic compound is chosen from the compounds of formula V in which the -Hy group is an alkyl group derived from a fatty acid chosen from the group consisting of linear fatty acids.

In one embodiment, the composition according to the invention is characterized in that the anionic compound is chosen from the compounds of formula V in which the -Hy group is an alkyl group derived from a saturated linear fatty acid chosen from the group consisting of lauric (dodecanoic) acid, myristic (tetradecanoic) acid, palmitic (hexadecanoic) acid, stearic (octadecanoic) acid, arachidic (eicosanoic) acid, behenic (docosanoic) acid, tricosanoic acid, lignoceric (tetracosanoic) acid, heptacosanoic acid, octacosanoic acid and melissic (tricontanoic) acid.

In one embodiment, the composition according to the invention is characterized in that the anionic compound is chosen from the compounds of formula V in which the -Hy group is an alkyl group derived from an unsaturated fatty acid.

In one embodiment, the composition according to the invention is characterized in that the anionic compound is chosen from the compounds of formula V in which the -Hy group is an alkyl group derived from an unsaturated fatty acid chosen from the group consisting of myristoleic ((Z)-tetradec-9-enoic) acid, palmitoleic ((Z)-hexadec-9-enoic) acid, oleic ((Z)-octadec-9-enoic) acid, elaidic ((E)-octadec-9-enoic) acid, linoleic ((9Z,12Z)-octadeca-9,12-dienoic) acid, alpha-linoleic ((9Z,12Z,15Z)-octadeca-9,12,15-trienoic) acid, arachidonic ((5Z,8Z,11Z,14Z)-octadeca-5,8,11,14-tetraenoic) acid, eicosapentaenoic ((5Z,8Z,11Z,14Z,17Z)-eicosa-5,8,11,14,17-pentaenoic) acid, erucic (13-docoenoic) acid and docosahexaenoic ((4Z,7Z,10Z,13Z,16Z,19Z)-docosa-4,7,10,13,16,19-hexaenoic) acid.

In one embodiment, the composition according to the invention is characterized in that the anionic compound is chosen from the compounds of formula V in which the -Hy group is an alkyl group derived from a bile acid and derivatives thereof.

In one embodiment, the composition according to the invention is characterized in that the anionic compound is chosen from the compounds of formula V in which the -Hy group is an alkyl group derived from a bile acid and derivatives thereof, chosen from the group consisting of cholic acid, dehydrocholic acid, deoxycholic acid and chenodeoxycholic acid.

In one embodiment, the composition according to the invention is characterized in that the anionic compound is chosen from the compounds of formula V in which the radical —NH-[E]-(o-[Hy])ₜ is such that t=1, —NH-E-(o-)ₜ is derived from ethylenediamine, o is a carbamate function and -Hy is derived from cholesterol.

In one embodiment, the composition according to the invention is characterized in that the anionic compound bearing carboxylate charges and hydrophobic radicals is chosen from the compounds of formula V in which at most one of —R₂, —R₃, —R₄ and —R₆ is a backbone formed from a discrete number u of between 1 and 7 (1≦u≦7) of identical or different saccharide units, substituted with at least one substituent R′.

In one embodiment, the composition according to the invention is characterized in that the anionic compound bearing carboxylate charges and hydrophobic radicals is chosen from the compounds of formula V in which at most one of —R₂, —R₃, —R₄ and —R₆ is a backbone formed from a discrete number u of between 1 and 7 (1≦u≦7) of identical or different saccharide units, substituted with at least one substituent R′, and the at least one substituent R′ is a radical -f-[A]-COOH, -A- and f being as defined above.

In one embodiment, the composition according to the invention is characterized in that the anionic compound bearing carboxylate charges and hydrophobic radicals is chosen from the compounds of formula V in which the identical or different saccharide units are chosen from the group consisting of hexoses, uronic acids, hexosamines and N-acylhexosamines.

In one embodiment, the composition according to the invention is characterized in that the identical or different hexoses are in cyclic form or in open reduced form.

In one embodiment, the composition according to the invention is characterized in that the hexoses are identical or different cyclic hexoses and are chosen from the group consisting of fructose, sorbose, tagatose, psicose, glucose, mannose, galactose, allose, altrose, talose, idose, gulose, fucose, fuculose and rhamnose.

In one embodiment, the composition according to the invention is characterized in that the hexoses are identical or different, open reduced hexoses chosen from the group consisting of mannitol, xylitol, sorbitol and galactitol (dulcitol).

In one embodiment, the composition according to the invention is characterized in that the identical or different uronic acids are in cyclic form or in open oxidized form.

In one embodiment, the composition according to the invention is characterized in that the identical or different uronic acids in cyclic form are chosen from the group consisting of glucuronic acid, iduronic acid and galacturonic acid.

In one embodiment, the composition according to the invention is characterized in that the identical or different uronic acids in open oxidized form are chosen from the group consisting of gluconic acid, glucaric acid and galactonic acid.

In one embodiment, the composition according to the invention is characterized in that the identical or different hexosamines are in cyclic form, in open reduced form or in open oxidized form.

In one embodiment, the composition according to the invention is characterized in that the identical or different hexosamines in cyclic form are chosen from the group consisting of glucosamine, galactosamine and mannosamine.

In one embodiment, the composition according to the invention is characterized in that the hexosamine in open reduced form is meglumine.

In one embodiment, the composition according to the invention is characterized in that the hexosamine in open oxidized form is glucosaminic acid.

In one embodiment, the composition according to the invention is characterized in that the identical or different N-acetylhexosamines are in cyclic form or in open reduced form.

In one embodiment, the composition according to the invention is characterized in that the identical or different N-acetylhexosamines in cyclic form are chosen from the group consisting of N-acetylglucosamine, N-acetylgalactosamine and N-acetylmannosamine.

In one embodiment, the composition according to the invention is characterized in that the anionic compound bearing carboxylate charges and hydrophobic radicals is chosen from the compounds of formula V in which u=7.

In one embodiment, the composition according to the invention is characterized in that the anionic compound bearing carboxylate charges and hydrophobic radicals is chosen from the compounds of formula V in which u=6.

In one embodiment, the composition according to the invention is characterized in that the anionic compound bearing carboxylate charges and hydrophobic radicals is chosen from the compounds of formula V in which u=5.

In one embodiment, the composition according to the invention is characterized in that the anionic compound bearing carboxylate charges and hydrophobic radicals is chosen from the compounds of formula V in which u=4.

In one embodiment, the composition according to the invention is characterized in that the anionic compound bearing carboxylate changes and hydrophobic radicals is chosen from the compounds of formula V in which u=3.

In one embodiment, the composition according to the invention is characterized in that the anionic compound bearing carboxylate charges and hydrophobic radicals is chosen from the compounds of formula V in which u=2.

In one embodiment, the composition according to the invention is characterized in that the anionic compound bearing carboxylate charges and hydrophobic radicals is chosen from the compounds of formula V in which u=1,

In one embodiment, the composition according to the invention is characterized in that the anionic compound bearing carboxylic changes and hydrophobic radicals is chosen from the compounds of formula V in which the glucosidic linkages of the radical formed from a discrete number u of saccharide units of the anionic compound are of 1,2, 1,3, 1,4 or 1,6 type, which may be identical or different.

In one embodiment, the composition according to the invention is characterized in that the anionic compound bearing carboxylic changes and hydrophobic radicals is chosen from the compounds of formula V in which the glucosidic linkages of the radical formed from a discrete number u of saccharide units of the anionic compound are of α and/or β type, which may be identical or different.

In one embodiment, the composition according to the invention is characterized in that the anionic compound bearing carboxylate charges and hydrophobic radicals is chosen from the compounds of formula V in which the radical —R₄ is a saccharide sequence such that u=2 end the glucosidic linkages are of 1,4 type.

In one embodiment, the composition according to the invention is characterized in that the anionic compound bearing carboxylate charges and hydrophobic radicals is chosen from the compounds of formula V in which the radical —R₄ is a saccharide sequence such that u=3 and the glucosidic linkages are 1,4 type.

In one embodiment, the composition according to the invention is characterized in that the anionic compound bearing carboxylate charges and hydrophobic radicals is chosen from the compounds of formula V in which the radical —R₄ is a saccharide sequence such that u=4 and the glucosidic linkages are of 1.4 type.

In one embodiment, the composition according to the invention is characterized in that the anionic compound bearing carboxylate charges and hydrophobic radicals is chosen from the compounds of formula V in which the radical —R₄ is a saccharide sequence such that u=5 and the glucosidic linkages are of 1,4 type.

In one embodiment, the composition according to the invention is characterized in that the anionic compound bearing carboxylate charges and hydrophobic radicals is chosen from the compounds of formula V in which the radical —R₄ is a saccharide sequence such that u=6 and the glucosidic linkages are of 1,4 type.

In one embodiment, the composition according to the invention is characterized in that the anionic compound bearing carboxylate charges and hydrophobic radicals is chosen from the compounds of formula V in which the radical —R₄ is a saccharide sequence such that u=7 and the glucosidic linkages are of 1,4 type.

In one embodiment, the composition according to the invention is characterized in that the backbone before substitution with radicals bearing carboxylate charges and hydrophobic radicals is composed of 3 saccharide units, the glucosidic linkages of which are of a 1,4 type, and is maltotriose.

In one embodiment, the composition according to the invention is characterized in that the backbone before substitution with radicals bearing carboxylate charges and hydrophobic radicals is composed of 5 saccharide units, the glucosidic linkages of which are of a 1,4 type, and is maltopentaose.

In one embodiment, the composition according to the invention is characterized in that the backbone before substitution with radicals bearing carboxylate charges and hydrophobic radicals is composed of 8 saccharide units, the glucosidic linkages of which are of a 1,4 type, and is maltooctaose.

In one embodiment, the composition according to the invention is characterized in that the anionic compound is chosen from the compounds of formula V in which —X— is the radical —CH₂—, —Z— is the radical —CH₂—, the radical —R₄ is a saccharide sequence such that u=7 and the saccharide linkages are of 1,4 type, the radical -A- is —CH₂ and f is an ether function.

In one embodiment, the composition according to the invention is characterized in that the anionic compound is chosen from the compounds of formula V in which —X— is the radical —CH₂—, —Z— is the radical —CH₂—, the radical —R₄ is a saccharide sequence such that u=7 and the glucosidic linkages are of 1,4 type, the radical -A- is —CH₂ and f is a function, and the radical —NH-[E]-(o-[Hy])ₜ is such that t=1, —NH-E-(o-)ₜ is derived from ethylenediamine, o is a carbamate function and -Hy is derived from cholesterol.

The anionic compounds bearing carboxylate charges and hydrophobic radicals according to formulae I, IV, V, IX, X and XI can be obtained by various synthesis routes, depending on whether they are in the isolated state or as a mixture.

The compounds can be obtained by random grafting of the carboxylate charges and/or hydrophobic radicals onto the saccharide backbone. Said compounds are then obtained in the form of a mixture.

In one embodiment, the anionic compounds bearing carboxylate charges and hydrophobic radicals are chosen from compounds of formulae I, IV, V, IX, X and XI, and they can be obtained by grafting of the carboxylate charges and/or hydrophobic radicals at precise positions on the saccharide units by means of a process which implements steps for protection/deprotection of the alcohol or carboxylic acid groups naturally borne by the backbone. This strategy results in selective, in particular, regioselective, grafting of the carboxylate charges and/or hydrophobic radicals onto the backbone. The protective groups include, without limitation, those described in the book Wuts, P G M et al., Greene's Protective Groups in Organic Synthesis 2007. Said compounds are then obtained in the isolated state.

The saccharide backbone can also be obtained by formation of glycosidic linkages between monosaccharide or oligosaccharide molecules using a chemical or enzymatic coupling strategy. The coupling strategies include those described in the publication Smoot, J T et al., Advances in Carbohydrate Chemistry and Biochemistry 2009, 62, 162-236 and in the book Lindhorst, T K, Essentials of Carbohydrate Chemistry and Biochemistry 2007, 157-208. The coupling reactions can be carried out in solution or on a solid support. The saccharide molecules before coupling may bear radicals of interest and/or be functionalized once randomly or regioselectively coupled to one another. Said compounds are then, respectively, obtained either in the form of a mixture, or in the isolated state.

Thus, by way of examples, the compounds according to the invention can be obtained according to one of the following processes:
random grafting of the carboxylate charges and/or hydrophobic radicals onto a saccharide backbone;
one or more steps of glycosylation between monosaccharide or oligo-saccharide molecules bearing carboxylate charges and/or hydrophobic radicals;
one or more steps of glycosylation between one or more monosaccharide or oligosaccharide molecules bearing carboxylate charges and/or hydrophobic radicals and one or more monosaccharide or oligosaccharide molecules;
one or more steps of introduction of protective groups onto alcohols or acids naturally borne by the saccharide backbone, followed by one or more reactions for grafting of the carboxylate charges and/or hydrophobic radicals and, finally, a step of removal of the protective groups;
one or more steps of glycosylation between one or more monosaccharide or oligosaccharide molecules bearing protective groups on alcohols or acids naturally borne by the saccharide backbone, one or more steps for grafting of carboxylate charges and/or hydrophobic radicals onto the backbone obtained, and then a step of removal of the protective groups;
one or more steps of glycosylation between one or more monosaccharide or oligosaccharide molecules bearing protective groups on alcohols or acids naturally borne by the saccharide backbone, and one or more monosaccharide or oligosaccharide molecules, one or more steps for grafting of carboxylate charges and/or hydrophobic radicals, and then a step of removal of the protective groups.

The compounds according to the invention, isolated or as a mixture, can be separated and/or purified in various ways, in particular after they have been obtained by means of the processes described above.

Mention may in particular be made of chromatographic methods, in particular those termed “preparative”, for instance:
flash chromatography, in particular on silica, and
chromatography of HPLC (high performance liquid chromatography) type, in particular RP-HPLC (reverse-phase HPLC).

Selected precipitation methods can also be used.

The expression “basal insulin, the isoelectric point of which is between 5.8 and 8.5” is intended to mean an insulin which is insoluble at pH 7 and the duration of action of which is between 8 and 24 hours or more in the standard diabetes models.

These basal insulins, the isoelectric point of which is between 5.8 and 8.5, are recombinant insulins of which the primary structure has been modified mainly by introducing basic amino acids such as arginine or lysine. They are described, for example, in the following patents, patent applications or publications WO 2003/053339, WO 2004/096854, U.S. Pat. No. 5,656,722 and U.S. Pat. No. 6,100,376, the content of which is incorporated by way of reference.

In one embodiment, the basal insulin, the isoelectric point of which is between 5.8 and 8.5, is insulin glargine.

In one embodiment, the compositions according to the invention comprise between 40 and 500 IU/ml of basal insulin, the isoelectric point of which is between 5.8 and 8.5.

In one embodiment, the compositions according to the invention comprise between 100 and 350 IU/ml of basal insulin, the isoelectric point of which is between 5.8 and 8.5.

In one embodiment, the compositions according to the invention comprise 40 IU/ml of basal insulin, the isoelectric point of which is between 5.8 and 8.5.

In one embodiment, the compositions according to the invention comprise 100 IU/ml (i.e. approximately 3.6 mg/ml) of basal insulin, the isoelectric point of which is between 5.8 and 8.5.

In one embodiment, the compositions according to the invention comprise 200 IU/ml of basal insulin, the isoelectric point of which is between 5.8 and 8.5.

In one embodiment, the compositions according to the invention comprise 300 IU/ml of basal insulin, the isoelectric point of which is between 5.8 and 8.5.

In one embodiment, the compositions according to the invention comprise 400 IU/ml of basal insulin, the isoelectric point of which is between 5.8 and 8.5.

In one embodiment, the compositions according to the invention comprise 500 IU/ml of basal insulin, the isoelectric point of which is between 5.8 and 8.5.

In one embodiment, the weight ratio between the basal insulin, the isoelectric point of which is between 5.8 and 8.5, and the anionic compound bearing carboxylate charges and hydrophobic radicals, that is to say anionic compound bearing carboxylate charges and hydrophobic radicals/basal insulin, is between 0.2 and 30.

In one embodiment, the weight ratio is between 0.2 and 15.

In one embodiment, the weight ratio is between 0.2 and 10.

In one embodiment, the weight ratio is between 0.2 and 4.

In one embodiment, the weight ratio is between 0.2 and 3.

In one embodiment, the weight ratio is between 0.2 and 2.

In one embodiment, the weight ratio is between 0.2 and 1.7.

In one embodiment, the weight ratio is between 0.6 and 1.7.

In one embodiment, the weight ratio is between 0.5 and 3.

In one embodiment, the concentration of anionic compound bearing carboxylate charges and hydrophobic radicals is at most 100 mg/ml.

In one embodiment, the concentration of anionic compound bearing carboxylate charges and hydrophobic radicals is at most 80 mg/ml.

In one embodiment, the concentration of anionic compound bearing carboxylate charges and hydrophobic radicals is at most 60 mg/ml.

In one embodiment, the concentration of anionic compound bearing carboxylate charges and hydrophobic radicals is at most 40 mg/ml.

In one embodiment, the concentration of anionic compound bearing carboxylate charges and hydrophobic radicals is at most 20 mg/ml.

In one embodiment, the concentration of anionic compound bearing carboxylate charges and hydrophobic radicals is at most 10 mg/ml.

In one embodiment, the concentration of anionic compound bearing carboxylate charges and hydrophobic radicals is at most 5 mg/ml.

In one embodiment, the compositions according to the invention also comprise a prandial insulin. The prandial insulins are soluble at pH 7.

The term “prandial insulin” is intended to mean an insulin termed fast-acting or “regular”.

The prandial insulins termed fast-acting insulins are insulins which must respond to the needs caused by the ingestion of proteins and carbohydrates during a meal; they must act in less than 30 minutes.

In one embodiment, the prandial insulin termed “regular” is human insulin.

In one embodiment, the insulin is a recombinant human insulin as described in the European Pharmacopeia and the American Pharmacopeia.

Human insulin is, for example, sold under the brand names Humulin® (ELI LILLY) and Novolin® (NOVO NORDISK).

The prandial insulins termed fast-acting are insulins which are obtained by recombination and the primary structure of which has been modified so as to reduce their action time.

In one embodiment, the prandial insulins termed fast-acting are chosen from the group comprising insulin lispro (Humalog®), insulin glulisine (Apidra®) and insulin aspart (NovoLog®).

In one embodiment, the prandial insulin is insulin lispro.

In one embodiment, the prandial insulin is insulin glulisine.

In one embodiment, the prandial insulin is insulin aspart.

In one embodiment, the compositions according to the invention comprise in total between 40 and 800 IU/ml of insulin with a combination of prandial insulin and basal insulin, the isoelectric point of which is between 5.8 and 8.5.

In one embodiment, the compositions according to the invention comprise in total between 40 and 500 IU/ml of insulin with a combination of prandial insulin and basal insulin, the isoelectric point of which is between 5.8 and 8.5.

In one embodiment, the compositions according to the invention comprise in total 800 IU/ml of insulin with a combination of prandial insulin and basal insulin, the isoelectric point of which is between 5.8 and 8.5.

In one embodiment, the compositions according to the invention comprise in total 700 IU/ml of insulin with a combination of prandial insulin and basal insulin, the isoelectric point of which is between 5.8 and 8.5.

In one embodiment, the compositions according to the invention comprise in total 600 IU/ml of insulin with a combination of prandial insulin and basal insulin, the isoelectric point of which is between 5.8 and 8.5.

In one embodiment, the compositions according to the invention comprise in total 500 IU/ml of insulin with a combination of prandial insulin and basal insulin, the isoelectric point of which is between 5.8 and 8.5.

In one embodiment, the compositions according to the invention comprise in total 400 IU/ml of insulin with a combination of prandial insulin and basal insulin, the isoelectric point of which is between 5.8 and 8.5.

In one embodiment, the compositions according to the invention comprise in total 300 IU/ml of insulin with a combination of prandial insulin and basal insulin, the isoelectric point of which is between 5.8 and 8.5.

In one embodiment, the compositions according to the invention comprise in total 200 IU/ml of insulin with a combination of prandial insulin and basal insulin, the isoelectric point of which is between 5.8 and 8.5.

In one embodiment, the compositions according to the invention comprise in total 100 IU/ml of insulin with a combination of prandial insulin and basal insulin, the isoelectric point of which is between 5.8 and 8.5.

In one embodiment, the compositions according to the invention comprise in total 40 IU/ml of insulin with a combination of prandial insulin and basal insulin, the isoelectric point of which is between 5.8 and 8.5.

The proportions between the basal insulin, the isoelectric point of which is between 5.8 and 8.5, and the prandial insulin are, for example, as a percentage, 25/75, 30/70, 40/60, 50/50, 60/40, 63/37, 70/30, 75/25, 80/20, 83/17, 90/10 for formulations as described above comprising from 40 to 800 IU/ml. However, any other proportion can be used.

In one embodiment, the proportions between the basal insulin, the isoelectric point of which is between 5.8 and 8.5, and the prandial insulin are 75/25.

In one embodiment, the proportions between the basal insulin, the isoelectric point of which is between 5.8 and 8.5, and the prandial insulin are 63/37.

In one embodiment, the compositions according to the invention also comprise a gut hormone.

The term “gut hormones” is intended to mean the hormones chosen from the group consisting of GLP-1 (Glucagon like peptide-1) and GIP (Glucose-dependent insulinotropic peptide), oxyntomodulin (a proglucagon derivative), peptide YY, amylin, cholecystokinin, pancreatic polypeptide (PP), ghrelin and enterostatin, analogs or derivatives thereof and/or pharmaceutically acceptable salts thereof.

In one embodiment, the gut hormones are GLP-1 analogs or derivatives chosen from the group consisting of exenatide or Byetta®, developed by ELI LILLY & CO and AMYLIN PHARMACEUTICALS, liraglutide or Victoza® developed by NOVO NORDISK, or lixisenatide or Lyxumia® developed by SANOFI-AVENTIS, analogs or derivatives thereof and pharmaceutically acceptable salts thereof.

In one embodiment, the gut hormone is exenatide or Byetta®, analogs or derivatives thereof and pharmaceutically acceptable salts thereof.

In one embodiment, the gut hormone is liraglutide or Victoza®, analogs or derivatives thereof and pharmaceutically acceptable salts thereof.

In one embodiment, the gut hormone is lixisenatide or Lyxumia®, analogs or derivatives thereof and pharmaceutically acceptable salts thereof.

The term “analog”, when it is used with reference to a peptide or a protein, is intended to mean a peptide or protein in which one or more constituent amino acid residues have been substituted with other amino acid residues and/or in which one or more constituent amino acid residues have been deleted and/or in which one or more constituent amino acid residues have been added. The percentage homology accepted for the present definition of an analog is 50%.

The term “derivative”, when it is used with reference to a peptide or a protein, is intended to mean a peptide or a protein or an analog chemically modified with a substituent which is not present in the reference peptide, protein or analog, i.e. a peptide or a protein which has been modified by creating covalent bonds, so as to introduce substituents.

In one embodiment, the substituent is chosen from the group consisting of fatty chains.

In one embodiment, the compositions according to the invention also comprise zinc salts at a concentration of between 0 and 5000 μM.

In one embodiment, the compositions according to the invention also comprise zinc salts at a concentration of between 0 and 4000 μM.

In one embodiment, the compositions according to the invention also comprise zinc salts at a concentration of between 0 and 3000 μM.

In one embodiment, the compositions according to the invention also comprise zinc salts at a concentration of between 0 and 2000 μM.

In one embodiment, the compositions according to the invention also comprise zinc salts at a concentration of between 0 and 1000 μM.

In one embodiment, the compositions according to the invention also comprise zinc salts at a concentration of between 50 and 600 μM.

In one embodiment, the compositions according to the invention also comprise zinc salts at a concentration of between 100 and 500 μM.

In one embodiment, the compositions according to the invention also comprise zinc salts at a concentration of between 200 and 500 μM.

In one embodiment, the compositions according to the invention also comprise buffers.

In one embodiment, the compositions according to the invention comprise buffers at concentrations of between 0 and 100 mM.

In one embodiment, the compositions according to the invention comprise buffers at concentrations of between 15 and 50 mM.

In one embodiment, the compositions according to the invention comprise a buffer chosen from the group consisting of a phosphate buffer, Tris(trishydroxymethylaminomethane) and sodium citrate.

In one embodiment, the buffer is sodium phosphate.

In one embodiment, the buffer is Tris(trishydroxymethylaminomethane).

In one embodiment, the buffer is sodium citrate.

In one embodiment, the compositions according to the invention also comprise preserving agents.

In one embodiment, the preserving agents are chosen from the group consisting of m-cresol and phenol, alone or as a mixture.

In one embodiment, the concentration of preserving agents is between 10 and 50 mM.

In one embodiment, the concentration of preserving agents is between 10 and 40 mM.

In one embodiment, the compositions according to the invention also comprise a surfactant.

In one embodiment, the surfactant is chosen from the group consisting of propylene glycol and polysorbate.

The compositions according to the invention may also comprise additives, such as tonicity agents.

In one embodiment, the tonicity agents are chosen from the group consisting of glycerol, sodium chloride, mannitol and glycine.

The compositions according to the invention may also comprise any excipients in accordance with the pharmacopeias which are compatible with the insulins used at the working concentrations.

The invention also relates to a pharmaceutical formulation as claimed in the invention, which is obtained by drying and/or lyophilization.

In the case of local and systemic release, the envisioned modes of administration are intravenous, subcutaneous, intradermal or intramuscular.

Transdermal, oral, nasal, vaginal, ocular, buccal and pulmonary administration routes are also envisioned.

The invention also relates to single-dose formulations at a pH of between 6.6 and 7.8 comprising a basal insulin, the isoelectric point of which is between 5.8 and 8.5.

The invention also relates to single-dose formulations at a pH of between 6.6 and 7.8 comprising a basal insulin, the isoelectric point of which is between 5.8 and 8.5, and a prandial insulin.

The invention also relates to single-dose formulations at a pH of between 6.6 and 7.8 comprising a basal insulin, the isoelectric point of which is between 5.8 and 8.5, and a gut hormone, as previously defined.

The invention also relates to single-dose formulations at a pH of between 6.6 and 7.8 comprising a basal insulin, the isoelectric point of which is between 5.8 and 8.5, a prandial insulin and a gut hormone, as previously defined.

The invention also relates to single-dose formulations at a pH of between 7 and 7.8 comprising a basal insulin, the isoelectric point of which is between 5.8 and 8.5.

The invention also relates to single-dose formulations at a pH of between 7 and 7.8 comprising a basal insulin, the isoelectric point of which is between 5.8 and 8.5, and a prandial insulin.

The invention also relates to single-dose formulations at a pH of between 7 and 7.8 comprising a basal insulin, the isoelectric point of which is between 5.8 and 8.5, and a gut hormone, as previously defined.

The invention also relates to single-dose formulations at a pH of between 7 and 7.8 comprising a basal insulin, the isoelectric point of which is between 5.8 and 8.5, a prandial insulin and a gut hormone, as previously defined.

In one embodiment, the single-dose formulations also comprise an anionic compound bearing carboxylate charges and hydrophobic radicals, as previously defined.

In one embodiment, the formulations are in the form of an injectable solution.

In one embodiment, the basal insulin, the isoelectric point of which is between 5.8 and 8.5, is insulin glargine.

In one embodiment, the prandial insulin is human insulin.

In one embodiment, the insulin is a recombinant human insulin as described in the European Pharmacopeia and the American Pharmacopeia.

In one embodiment, the prandial insulin is chosen from the group comprising insulin lispro (Humalog®), insulin glulisine (Apidra®) and insulin aspart (NovoLog).

In one embodiment, the prandial insulin is insulin lispro.

In one embodiment, the prandial insulin is insulin glulisine.

In one embodiment, the prandial insulin is insulin aspart.

In one embodiment, the GLP-1 or GLP-1 analog or derivative is chosen from the group comprising exenatide (Byetta®), liraglutide (Victoza®) and lixisenatide (Lyxumia®), or a derivative thereof.

In one embodiment, the gut hormone is exenatide.

In one embodiment, the gut hormone is liraglutide.

In one embodiment, the gut hormone is lixisenatide.

The solubilization, at a pH of between 6.6 and 7.8, of the basal insulins, the isoelectric point of which is between 5.8 and 8.5, by the anionic compounds bearing carboxylate charges and hydrophobic radicals of formulae I to V and VIII to XI, can be simply observed and controlled, with the naked eye, through a change in appearance of the solution.

The solubilization, at a pH of between 7 and 7.8, of the basal insulins, the isoelectric point of which is between 5.8 and 8.5, by the anionic compounds bearing carboxylate charges and hydrophobic radicals of formulae I to V and VIII to XI, can be simply observed and controlled, with the naked eye, through a change in appearance of the solution.

Moreover and just as importantly, the applicant has been able to verify that a basal insulin, the isoelectric point of which is between 5.8 and 8.5, solubilized at a pH of between 6.6 and 7.8, in the presence of an anionic compound bearing carboxylate charges and hydrophobic radicals of formulae I to V and VIII to XI, retains a slow insulin action, whether alone or in combination with a prandial insulin or a gut hormone.

The applicant has also been able to verify that a prandial insulin mixed at a pH of between 6.6 and 7.8 in the presence of an anionic compound bearing carboxylate charges and hydrophobic radicals of formulae I to V and VIII to XI and of a basal insulin, the isoelectric point of which is between 5.8 and 8.5, retains a fast insulin action.

The preparation of a composition according to the invention has the advantage of being able to be carried out by simply mixing an aqueous solution of basal insulin, the isoelectric point of which is between 5.8 and 8.5, and an anionic compound bearing carboxylate charges and hydrophobic radicals of formulae I to V and VIII to XI, in aqueous solution or in lyophilized form. If necessary, the pH of the preparation is adjusted to pH 7.

The preparation of a composition according to the invention has the advantage of being able to be carried out by simply mixing an aqueous solution of basal insulin, the isoelectric point of which is between 5.8 and 8.5, a solution of prandial insulin, and an anionic compound bearing carboxylate charges and hydrophobic radicals of formulae I to V and VIII to XI, in aqueous solution or in lyophilized form. If necessary, the pH of the preparation is adjusted to pH 7.

The preparation of a composition according to the invention has the advantage of being able to be carried out by simply mixing an aqueous solution of basal insulin, the isoelectric point of which is between 5.8 and 8.5, a solution of GLP-1 or a GLP-1 analog or derivative, and an anionic compound bearing carboxylate charges and hydrophobic radicals of formulae I to V and VIII to XI, in aqueous solution or in lyophilized form. If necessary, the pH of the preparation is adjusted to pH 7.

The preparation of a composition according to the invention has the advantage of being able to be carried out by simply mixing an aqueous solution of basal insulin, the isoelectric point of which is between 5.8 and 8.5, a solution of prandial insulin, a solution of GLP-1 or a GLP-1 analog or derivative, and an anionic compound bearing carboxylate charges and hydrophobic radicals of formulae I to V and VIII to XI, in aqueous solution or in lyophilized form. If necessary, the pH of the preparation is adjusted to pH 7.

In one embodiment, the mixture of basal insulin and anionic compound bearing carboxylate charges and hydrophobic radicals is concentrated by ultrafiltration before mixing with the prandial insulin in aqueous solution or in lyophilized form.

### DESCRIPTION OF THE FIGURES

FIG. 1: Curves of mean blood glucose level±standard error of the mean for the simultaneous administrations of Humalog® (100 IU/ml, 0.05 IU/kg) and Lantus® (100 IU/ml, 0.15 IU/kg) in comparison with the administration of a formulation as claimed in the invention, described in example B33 (400 IU/ml, 0.4 IU/kg).

FIG. 2: Curves of mean blood glucose level±standard error of the mean for the simultaneous administrations of Humalog® (100 IU/ml, 0.05 IU/kg) and Lantus® (100 IU/ml, 0.15 IU/kg) in comparison with the administration of a formulation as claimed in the invention, described in example B40 (400 IU/ml, 0.4 IU/kg).

FIG. 3: Curves of mean blood glucose level±standard error of the mean for the simultaneous administrations of Humalog® (100 IU/ml, 0.075 IU/kg) and Lantus® (100 IU/ml, 0.225 IU/kg) in comparison with the administration of a formulation as claimed in the invention, described in example B48 (400 IU/ml, 0.3 IU/kg).

FIG. 4: Curves of mean blood glucose level±standard error of the mean for the simultaneous administrations of Humalog® (100 IU/ml, 0.075 IU/kg) and Lantus® (100 IU/ml, 0.225 IU/kg) in comparison with the administration of a formulation as claimed in the invention, described in example B47 (400 IU/ml, 0.3 IU/kg).

Examples:
Formula IV

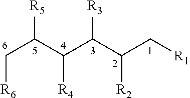

| | | R₁, R₂, R₃, R₅, R₆ and R′ | | |
|---|---|---|---|---|
| | | Radicals R₁, R₂, | | |
| | | R₃, R₅, R₆ and R′ | | |
| | | different from: | Radical -f-[A]-COOH (at | |
| Com- | | -f-[A]-COOH, or | least one of R₁, R₂, R₃, | |
| pound | R₄ | -g-[B]-(k-[D])ₚ) | R₅, R₆ and R′) | Radical -g-[B]-(k-[D])ₚ (at least one of R₁, R₂, R₃, R₅, R₆ and R′) |
| AA1 | 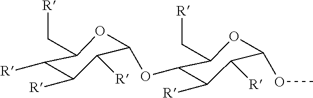 | —OH | 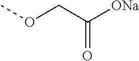 | 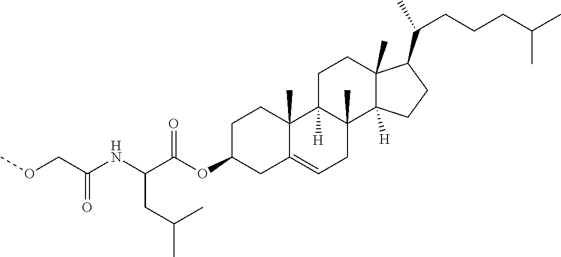 |
| AA2 | 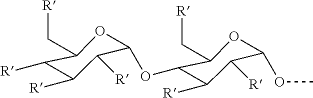 | —OH | 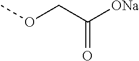 | 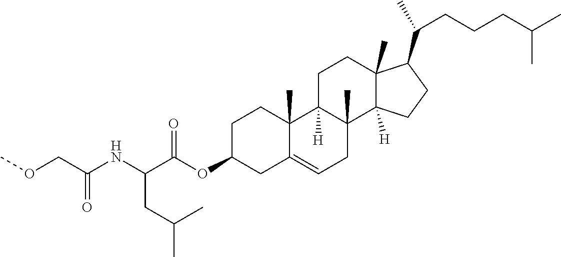 |
| AA3 | 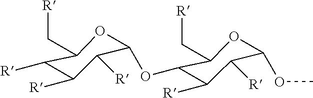 | —OH | 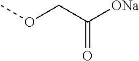 | 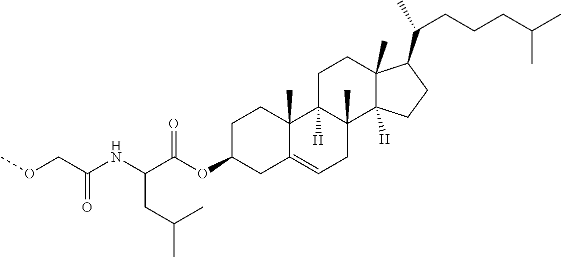 |
| AA4 | 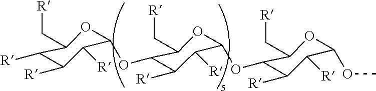 | —OH | 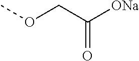 | 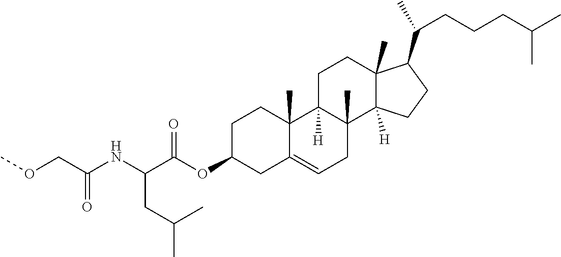 |
| AA5 | 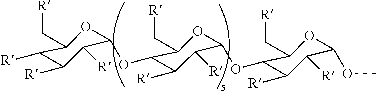 | —OH | 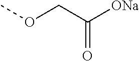 | 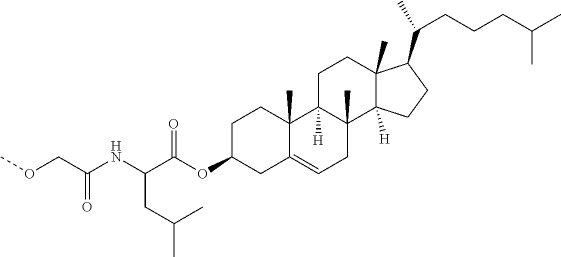 |
| AA6 | 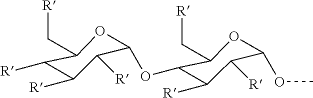 | —OH | 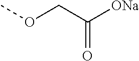 | 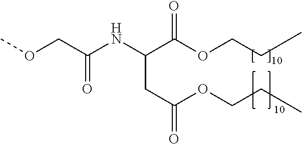 |
| AA7 | 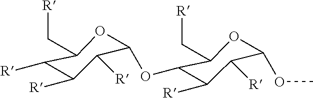 | —OH | 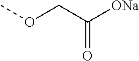 | 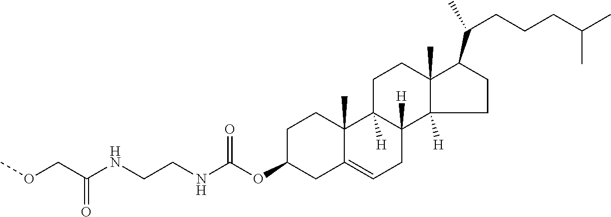 |
| AA8 | 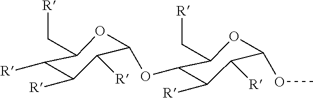 | —OH | 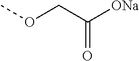 | 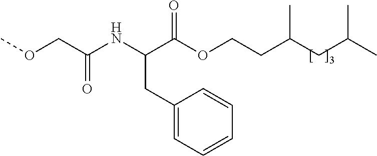 |
| AA9 | 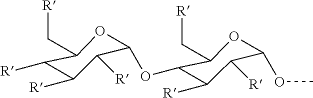 | —OH | 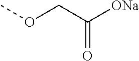 | 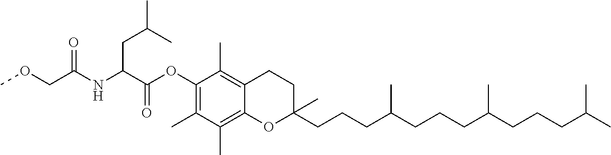 |

Formula II

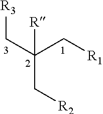

| Com- | | | |
|---|---|---|---|
| pound | Backbone | Radicals R₁, R₂ and R₃ | Radical R″ |
| AB1 | 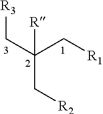 | 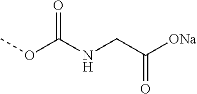 | 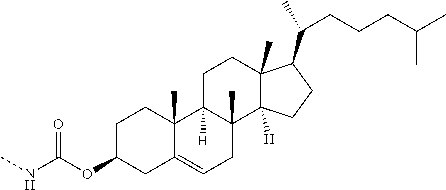 |
| AB2 | 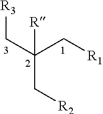 | 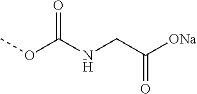 | 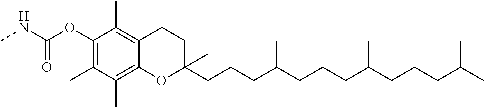 |
| AB3 | 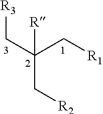 | 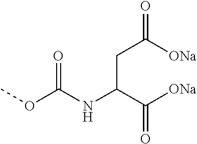 | 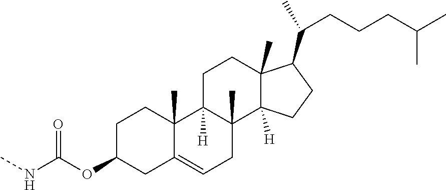 |
| AB4 | 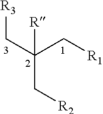 | 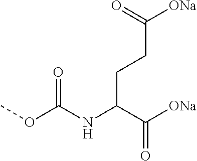 | 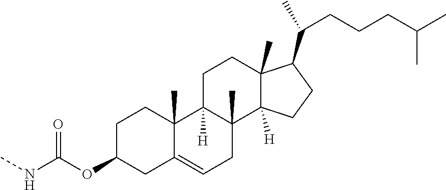 |

Formula III

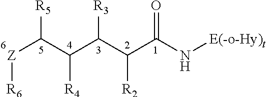

| Com- | | | | |
|---|---|---|---|---|
| pound | Backbone | Z | Radicals R₂, R₃, R₄, R₅ and R₆ | Radical E-(o-Hy)ₜ |
| AC1 | 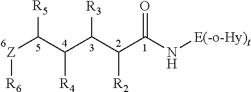 | CH₂ | 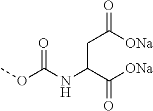 | 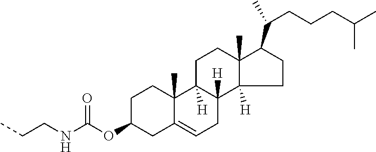 |

### Part A

### Synthesis of Hydrophobized Anionic Molecules

### Example AA1

### Sodium Maltotriosemethylcarboxylate Functionalized with Cholesteryl Leucinate

### Hydrophobized Anionic Molecule AA1

To 8 g (143 mmol of hydroxyl functions) of maltotriose (CarboSynth) dissolved in water at 65° C. is added 0.6 g (16 mmol) of sodium borohydride. After stirring for 30 minutes, 28 g (238 mmol) of sodium chloroacetate are added. To this solution are then added dropwise 24 mL of 10 N NaOH (240 mmol), and the mixture is then heated at 65° C. for 90 minutes. 16.6 g (143 mmol) of sodium chloroacetate are then added to the reaction mixture, along with dropwise addition of 14 mL of 10N

NaOH (140 mmol). After heating for 1 hour, the mixture is diluted with water, neutralized with acetic acid and then purified by ultrafiltration on a 1 kDa PES membrane against water. The hydrophobized molecule concentration of the final solution is determined on the dry extract, and an acid/base titration in a 50/50 (V/V) water/acetone mixture is then performed to determine the degree of substitution with sodium methylcarboxylate.

According to the dry extract: [compound]=32.9 mg/g

According to the acid/base titration, the degree of substitution with sodium methylcarboxylate is 1.84 per saccharide unit.

The sodium maltotriosemethylcarboxylate solution is acidified on a Purolite resin (anionic) to obtain maltotriosemethylcarboxylic acid, which is then lyophilized for 18 hours.

The cholesteryl leucinate, para-toluenesulfonic acid salt is prepared from cholesterol and leucine according to the process described in U.S. Pat. No. 4,826,818 (Kenji M., et al.).

10 g of maltotriosemethylcarboxylic acid (63 mmol of methylcarboxylic acid functions) are dissolved in DMF (40 g/l) and then cooled to 0° C. A mixture of cholesteryl leucinate, para-toluenesulfonic acid salt (2.3 g; 3 mmol) in DMF is prepared. 0.4 g of triethylamine (3 mmol) is added to this mixture. Once the mixture is at 0° C., a solution of NMM (1.9 g, 19 mmol) and of EtOCOCl (2.1 g; 19 mmol) is added. After 10 min, the cholesteryl leucinate solution is added and the mixture is stirred at 10° C. The mixture is then heated to 50° C. An aqueous solution of imidazole (150 g/l) is added and the medium is diluted with water. The resulting solution is purified by ultrafiltration on 1 kDa PES membranes against 0.01 N NaOH, 0.9% NaCl and water. The hydrophobized molecule concentration of the final solution is determined on the dry extract. A sample of solution is lyophilized and analyzed by ¹H NMR in D₂O/NaOD to determine the degree of substitution with methylcarboxylates grafted with cholesteryl leucinate.

According to the dry extract: [Hydrophobized anionic molecule AA1]=10.1 mg/g

According to the ¹H NMR: the degree of substitution with methylcarboxylates grafted with cholesteryl leucinate is 0.08.

### Example AA2

### Sodium Maltotriosemethylcarboxylate Functionalized with Cholesteryl Leucinate

### Hydrophobized Anionic Molecule AA2

Via a process similar to that described in the preparation of the hydrophobized anionic molecule AA1, a sodium maltotriosemethylcarboxylate characterized by a degree of substitution with sodium methylcarboxylate of 1.62 is functionalized with cholesteryl leucinate.

According to the dry extract: [Hydrophobized anionic molecule AA2]=29.4 mg/g

According to the ¹H NMR: the degree of substitution with methylcarboxylates grafted with cholesteryl leucinate is 0.29.

### Example AA3

### Sodium Maltotriosemethylcarboxylate Functionalized with Cholesteryl Leucinate

### Hydrophobized Anionic Molecule AA3

Via a process similar to that described in the preparation of the hydrophobized molecule AA1, 8 g of sodium maltotriosemethylcarboxylate characterized by a degree of substitution with sodium methylcarboxylate of 1.76 are synthesized and lyophilized. 8 g (58 mmol of hydroxyl functions) of the lyophilizate and 15 g (129 mmol) of sodium chloroacetate are dissolved in water at 65° C. To this solution are added dropwise 13 ml of 10 N NaOH (13 mmol) and the mixture is then heated at 65° C. for 90 minutes. 9 g (78 mmol) of chloroacetate are than added to the reaction medium, along with dropwise addition of 8 ml of 10N NaOH (80 mmol). After heating for 1 hour, the mixture is diluted with water, neutralized with acetic acid and then purified by ultrafiltration on a 1 kDa PES membrane against water. The compound concentration of the final solution is determined on the dry extract, and an acid/base titration in a 50/50 (V/V) water/acetone mixture is then performed to determine the degree of substitution with sodium methylcarboxylate.

According to the dry extract: [compound]=11.7 mg/g

According to the acid/base titration, the degree of substitution with sodium methylcarboxylate is 3.30.

The sodium maltotriosemethylcarboxylate solution is acidified on a Purolite resin (anionic) to obtain maltotriosemethylcarboxylic acid, which is then lyophilized for 18 hours.

Via a process similar to that described for the preparation of the hydrophobized molecule AA1, a sodium maltotriosemethylcarboxylate functionalized with cholesteryl leucinate is obtained.

According to the dry extract: [Hydrophobized anionic molecule AA3]=13.1 mg/g

According to the ¹H NMR: the degree of substitution with methylcarboxylates grafted with cholesteryl leucinate is 0.29.

### Example AA4

### Sodium Maltopentaosemethylcarboxylate Functionalized with Cholesteryl Leucinate

### Hydrophobized Anionic Molecule AA4

Via a process similar to that described in the preparation of the hydrophobized molecule AA1 from maltopentaose (CarboSynth), 10 g of maltopentaosemethylcarboxylic acid characterized by a degree of substitution with sodium methylcarboxylate of 1.75 are synthesized and lyophilized.

Via a process similar to that described in the preparation of the hydrophobized molecule AA1, a sodium maltopentaosemethylcarboxylate functionalized with cholesteryl leucinate is obtained.

According to the dry extract: [Hydrophobized anionic molecule AA4]=10.9 mg/g

According to the ¹H NMR: the degree of substitution with methylcarboxylates grafted with cholesteryl leucinate is 0.14.

Hydrophobized Molecule AA5: Sodium Maltooctaosemethylcarboxylate Functionalized with Cholesteryl Leucinate

### Hydrophobized Anionic Molecule AA5

Via a process inspired by that described in the preparation of the hydrophobized molecule AA1, 10 g of maltooctaosemethylcarboxylic acid characterized by a degree of substitution with sodium methylcarboxylate of 1.2 are synthesized and lyophilized.

Via a process similar to that described in the preparation of the hydrophobized molecule AA1, a sodium maltooctaosemethylcarboxylate functionalized with cholesteryl leucinate is obtained.

According to the dry extract: [Hydrophobized anionic molecule AA5]=14.7 mg/g

According to the ¹H NMR: the degree of substitution with methylcarboxylates grafted with cholesteryl leucinate is 0.09.

### Example AA6

### Sodium Maltotriosemethylcarboxylate Functionalized with Dilauryl Aspartate

### Hydrophobized Anionic Molecule AA6

Dilauryl aspartate, para-toluenesulphonic acid salt, is prepared from dodecanol and aspartic acid according to the process described in U.S. Pat. No. 4,826,818 (Kenji M. et al.).

Via a process inspired by that described in the preparation of the hydrophobized molecule AA3, 10 g of maltotriosemethylcarboxylic acid having a degree of substitution with methylcarboxylic acid of 2.73 per glucoside unit are obtained and then lyophilized.

Via a process similar to that described in the preparation of the hydrophobized anionic molecule AA1, a sodium maltotriosemethylcarboxylate characterized by a degree of substitution with sodium methylcarboxylate of 2.73 is functionalized with dilauryl aspartate in DMF. The medium is diluted with water and the solution obtained is then purified by dialysis on a 3.5 kDa cellulose membrane against a 150 mM NaHCO₃/Na₂CO₃ buffer, pH 10.4, 0.9% NaCl and water. The compound concentration of the final solution is determined by dry extract. A sample of solution is lyophilized and analyzed by ¹H NMR in D₂O to determine the degree of substitution with methyl-carboxylates functionalized with dilauryl aspartate.

According to the dry extract: [hydrophobized anionic molecule AA6]=3.4 mg/g

According to the ¹H NMR: the degree of substitution with methylcarboxylates functionalized with dilauryl aspartate is 0.36.

The degree of substitution with sodium methylcarboxylates per glucoside unit is 2.37.

### Example AA7

### Sodium Maltotriosemethylcarboxylate Functionalized with Cholesteryl 2-aminoethylcarbamate

### Hydrophobized Anionic Molecule AA7

Cholesteryl 2-aminoethylcarbamate, hydrochloric acid salt, is prepared according to the process described in patent WO 2010/053140 (Akiyoshi, K et al.). Via a process similar to that described in the preparation of the hydrophobized anionic molecule AA6, a sodium maltotriosemethylcarboxylate, characterized by a degree of substitution with sodium methylcarboxylate of 2.73, is functionalized with cholesteryl 2-aminoethylcarbamate.

According to the dry extract: [hydrophobized anionic molecule AA7]=2.9 mg/g

According to the ¹H NMR: the degree of substitution with methylcarboxylates functionalized with cholesteryl 2-aminoethylcarbamate is 0.28.

The degree of substitution with sodium methylcarboxylates per glucoside unit is 2.45.

### Example AA8

### Sodium Maltotriosemethylcarboxylate Functionalized with 3,7-dimethyl-octanoyl Phenylalaninate

### Hydrophobized Anionic Molecule AA8

3,7-Dimethyloctanoyl phenylalaninate, para-toluenesulphonic acid salt, is prepared from 3,7-dimethyloctan-1-ol and L-phenylalanine according to the process described in U.S. Pat. No. 4,826,818 (Kenji et al.).

Via a process similar to that described in the preparation of the hydrophobized anionic molecule AA1, a sodium maltotriosemethylcarboxylate, characterized by a degree of substitution with sodium methylcarboxylate of 1.64, is functionalized with 3,7-dimethyloctanoyl phenylalaninate.

According to the dry extract: [hydrophobized anionic molecule AA8]=3.3 mg/g

According to the ¹H NMR: the degree of substitution with methylcarboxylates functionalized with 3,7-dimethyloctanoyl phenylalaninate is 0.39.

The degree of substitution with sodium methylcarboxylates per glucoside unit is 1.25.

### Example AA9

### Sodium Maltotriosemethylcarboxylate Functionalized with (±)-α-tocopheryl Leucinate

### Hydrophobized Anionic Molecule AA9

(±)-α-Tocopheryl leucinate, hydrochloric acid salt, is obtained according to the process described in the publication Takata, J et al., Journal of Pharmaceutical Sciences 1995, 84(1), 96-100.

Via a process similar to that described in the preparation of the hydrophobized anionic molecule AA3, a sodium maltotriosemethylcarboxylate, characterized by a degree of substitution with sodium methylcarboxylate of 1.76, is functionalized with (±)-α-tocopheryl leucinate.

According to the dry extract: [hydrophobized anionic molecule AA9]=12.9 mg/g

According to the ¹H NMR: the degree of substitution with methylcarboxylates functionalized with (±)-α-tocopheryl leucinate is 0.26.

The degree of substitution with sodium methylcarboxylates per glucoside unit is 1.50.

### Example AB1

### Hydrophobized Anionic Molecule AB1

### Molecule 1: Product Obtained by Reaction Between tris(hydroxymethyl)aminomethane and Cholesteryl Chloroformate

Under argon, cholesteryl chloroformate (Alfa-Aesar) (20 g; 44.5 mmol) is added portionwise, over the course of 20 minutes, to a suspension of tris(hydroxymethyl)aminomethane (Tris, Sigma-Aldrich) (10.8 g; 89 mmol) in dimethylacetamide (180 ml). After stirring for 2 h, the medium is poured into 900 ml of water at pH 2-3. The white precipitate obtained is filtered off on a sinter funnel, rinsed with water and dried under vacuum in the presence of P₂O₅ for 3 days to give a white solid.

Yield: 23 g (97%)

¹H NMR (DMSO-d₆, ppm): 0.65 (3H); 0.80-1.50 (34H); 1.70-2.40 (6H); 3.52 (6H); 4.27 (1H); 5.33 (1H); 6.03 (1H).

### Molecule 2: Product Obtained by Reaction Between Molecule 1 and ethyl isocyanoacetate

Under argon, ethyl isocyanoacetate (TCI) (9.6 ml; 83.8 mmol) and 1,4-diazabicyclo[2.2.2]octane (DABCO) (1.2 g; 10.5 mmol) are added to a suspension of molecule 1 (14 g; 26.2 mmol) in toluene (525 ml). After heating at 110° C. for 5 h, the medium is concentrated under vacuum. The mixture obtained is taken up in dichloromethane and washed with 1 N HCl. After drying over Na₂SO₄, the organic phase is concentrated under vacuum to give a yellow oil which is purified by flash chromatography (dichloromethane/ethyl acetate). The desired product is obtained in the form of a white foam.

Yield: 20.2 g (84%)

¹H NMR (CDCl₃, ppm): 0.67 (3H); 0.80-1.70 (44H); 1.75-2.10 (4H); 2.20-2.40 (2H); 3.90-3.95 (6H); 4.20-4.25 (6H); 4.44 (6H); 5.17 (0.5H); 5.37 (1H); 5.78 (0.5H); 5.8-6.0 (3H).

LC/MS (ESI): 938.8 ([M+NH₄]⁺); ([M+NH₄]⁺ calculated: 939.1).

### Hydrophobized Anionic Molecule AB1

A suspension of molecule 2 (10.1 g; 10.96 mmol) in an ethanol/water mixture (110 ml/90 ml) is treated with 2 N NaOH (22 ml; 44 mmol) and then stirred at ambient temperature overnight. The ethanol is then evaporated off under vacuum and then 30 ml of 2 N HCl are gradually added to the medium, causing the formation of a white precipitate. The mixture is then extracted with ethyl acetate and the organic phase is then dried over Na₂SO₄, filtered and concentrated under vacuum to give a whitish solid. After trituration from pentane, filtration and drying under vacuum, a white solid corresponding to the hydrophobized molecule AB1 in acid form is obtained.

Yield: 7.4 g (81%)

¹H NMR (DMSO-d₆, ppm): 0.65 (3H); 0.80-1.70 (35H); 1.75-2.10 (4H); 2.10-2.35 (2H); 3.64 (6H); 4.15-4.45 (6H); 5.35 (1H); 6.9-7.2 (1.5H); 7.49 (2.5H); 11.75-13.5 (sh, 1.6H).

LC/MS (ESI): 835.8; (calculated: 836.9).

The hydrophobized molecule AB1 in acid form is dissolved in water and the solution is neutralized by gradually adding 10 N sodium hydroxide to give an aqueous solution of hydrophobized molecule AB1 which is then lyophilized.

¹H NMR (MeOD, ppm): 0.70 (3H); 0.80-1.70 (35H); 1.77-2.15 (4H); 2.25-2.40 (2H); 3.64 (6H); 4.42 (6H); 5.35 (1H).

### Example AB2

### Hydrophobized Anionic Molecule AB2

### Molecule 3: Carbamic Acid N-[2-hydroxy-1,1-bis(hydroxymethyl)ethyl]-1,1-dimethylethyl ester

Molecule 3 is obtained according to the process described in patent WO2008/30119, starting from 20 g of Tris.

Yield: 33.2 g (88%)

¹H NMR (DMSO-D₆, ppm): 1.37 (9H); 3.50 (6H); 4.48 (3H); 5.76 (1H).

### Molecule 4: Product Obtained by Reaction Between Molecule 3 and ethyl isocyanoacetate

Under argon, ethyl isocyanoacetate (9.0 ml; 79 mmol) and DABCO (1.1 g; 9.9 mmol) are added to a suspension of molecule 3 (5.46 g; 24.7 mmol) in toluene (210 ml). After heating at 110° C. for 5 h, the medium is concentrated under vacuum. The residue obtained is taken up in dichloromethane and washed with 1 N HCl. After drying over Na₂SO₄, the organic phase is concentrated under vacuum to give a translucent paste.

Yield: 16 g (quantitative)

¹H NMR (CDCl₃, ppm): 1.28 (9H); 1.42 (9H); 3.92 (6H); 4.17 (6H); 4.42 (6H); 5.20 (0.5H); 5.52 (0.5H); 5.90 (3H).

### Molecule 5: Hydrochloride Salt of the Product Obtained by Deprotection of the Boc Group of Molecule 4

Under argon, a solution of molecule 4 (15 g; 24.7 mmol) in dichloromethane (82 ml) at 0° C. is treated with HCl (4 M) in dioxane (62 ml). The reaction medium is stirred at 0° C. and then overnight at ambient temperature. The precipitate is recovered by filtration, triturated from diethyl ether and than cyclohexane, filtered and dried under vacuum to give a whitish solid.

Yield: 13.4 g (quantitative)

¹H NMR (CDCl₃, ppm): 1.26 (9H); 3.94 (6H); 4.13 (6H); 4.37 (6H); 6.57 (3H); 8.92 (3H).

### Molecule 6: Isocyanate Derived from Molecule 5

Triphosgene (2.34 g; 7.9 mmol) is added in one step to a solution of molecule 5 (12.9 g; 23.7 mmol) in dichloromethane (130 ml) and a saturated aqueous solution of NaHCO₃ (130 ml) at 0° c. After stirring at 0° C. for 1 h, the medium is transferred into a separating funnel and the organic phase is extracted with dichloromethane then dried over Na₂SO₄. After concentration under vacuum, a colorless oil is obtained.

Yield: 12.04 g (95%)

¹H NMR (CDCl₃, ppm): 1.28 (9H); 3.96 (6H); 4.20-4.40 (12H); 5.70 (3H).

### Molecule 7: Product Obtained by Reaction Between Molecule 6 and D-alpha-tocopherol

DABCO (1 g; 9 mmol) is added to a solution, under argon, of D-alpha-tocopherol (racemic Sigma-Aldrich) (9.7 g; 22.5 mmol) and molecule 6 (12 g; 22.5 mmol) in toluene (210 ml). The mixture a reused for 6 h and then left at ambient temperature overnight. After evaporation of the toluene, the oil obtained is purified by flash chromatography (dichloromethane/ethyl acetate).

Yield; 10.3 g (47%)

¹H NMR (CDCl₃, ppm): 0.75-0.90 (12H); 0.95-1.48 (30H) 1.45-1.65 (3H); 1.70-1.80 (2H); 1.90-2.15 (9H); 2.57 (2H); 3.94 (6H); 4.15-4.25 (6H); 4.50 (6H); 6.23 (3H); 6.60 (1H).

### Hydrophobized Anionic Molecule AB2

2 N sodium hydroxide (21 ml) is added to a solution of molecule 7 (10.22 g; 10.5 mmol) dissolved in a methanol/water mixture (110 ml/90 ml). After stirring at ambient temperature for 4 h, the medium is precipitated by adding 2 N HCl. The precipitate is filtered off and washed with water. The hydrophobized anionic molecule AB2 acid form obtained is taken up in water and the solution obtained is lyophilized.

Yield: 10 g (quantitative)

¹NMR (DMSO-d₆, ppm): 0.75-0.90 (12H); 0.95-1.60 (24H); 1.74 (2H); 1.90-2.05 (9H); 2.57 (2H); 3.65 (6H); 4.23 (6H); 7.11 (0.5H); 7.49 (2.5H); 7.72 (1H); 12.60 (3H).

LC/MS (ESI): $898.7 ([M+NH₄]⁺); ([M+NH₄]⁺ calculated: 899.2).

The hydrophobized anionic molecule AB2 in acid form is dissolved in water and the solution is neutralized by gradually adding 10 N sodium hydroxide to give an aqueous solution of hydrophobized anionic molecule AB2 which is then lyophilized.

¹H NMR (MeOD, ppm): 0.75-0.90 (12H); 1.00-1.70 (24H); 1.75-1.90 (2H); 1.95-2.20 (9H); 2.65 (2H); 3.75 (6H); 4.48 (6H).

### Example AB3

### Hydrophobized Anionic Molecule AB3

### Molecule 8: Hydrochloride Salt of L-aspartic Acid 1,4-dimethyl Ester

Thionyl chloride (32.7 ml) is added dropwise, for 1 h, to a solution of methanol (100 ml) at −8° C. without the temperature of the medium exceeding 0° C. After stirring of the medium while cold for 15 min, the cooling bath is removed and L-aspartic acid (Sigma-Aldrich) (30 g; 225 mmol) is added at ambient temperature. After stirring of the medium at ambient temperature overnight, the medium is concentrated under vacuum and co-evaporated with toluene (3 times) to give a beige solid.

Yield: 44.5 g (quantitative)

¹H NMR (DMSO-d₆; ppm): 3.05 (2H); 3.65 (3H); 3.73 (3H); 4.32 (1H); 8.81 (3H).

### Molecule 9: L-aspartic Acid 1,4-dimethyl ester isocyanate

Triphosgene (22.3 g; 75.3 mmol) is added in one step to a solution of molecule 8 (44.5 g; 225 mmol) in dichloromethane (900 ml) and a saturated aqueous solution of NaHCO₃ (900 ml) at 0° C. After stirring at 0° C. for 1 h, the medium is transferred into a separating funnel and the organic phase is extracted with dichloromethane then dried over Na₂SO₄. After concentration under vacuum and then distillation under vacuum, a colorless oil is obtained.

Yield: 39.4 g (93%)

¹H NMR (CDCl₃, ppm): 2.85 (2H); 3.74 (3H); 3.84 (3H); 4.41 (1H).

### Molecule 10: Product Obtained by Reaction Between Molecule 3 and Molecule 9

A suspension of molecule 3 (6 g; 27.1 mmol) in toluene (120 ml) is treated successively with DABCO (1.2 g) 10.84 mmol) and molecule 9 (18.3 g; 97.6 mmol). The mixture is added at 90° C. for 45 min and then concentrated under vacuum. The residue is purified by DCVC (Dry Column Vacuum Chromatography) (dichloromethane/ethyl acetate) to obtain a very pale yellow oil.

Yield: 21.2 g (quantitative)

¹H NMR (CDCl₃, ppm): 1.42 (9H); 2.75-3.20 (6H); 3.70 (9H); 3.75 (9H); 4.25-4.50 (6H); 4.55-4.70 (3H); 5.25 (1H); 5.58 (0.5H); 5.96 (2.5H).

LC/MS (ESI): 800.5 ([M+NH₄]⁺); ([M+NH₄]⁺ calculated: 800.7).

### Molecule 11: Hydrochloride Salt of the Product Obtained by the Protection of the Boc Group of Molecule 10

Via a process similar to that used to prepare molecule 5, molecule 11 is obtained in the form of a pale yellow oil.

Yield: quantitative

¹H NMR (DMSO-d₆, ppm): 2.70-2.90 (6H); 3.50-3.75 (18H); 4.15-4.30 (6H); 4.45-4.55 (3H); 4.85 (0.5H); 6.77 (0.5H); 7.44 (0.5H); 7.67 (2.5H); 8.80 (3H).

### Molecule 12: Product Obtained by Reaction Between Molecule 11 and Cholesteryl Chloroformate

Cholesteryl chloroformate (8.7 g; 19.2 mmol) and N,N-diisopropylethylamine

(DIPEA) (3.3 ml; 19.2 mmol) are added to a solution of molecule 11 (11 g; 16 mmol) in dichloromethane (60 ml). After stirring for 5 h at ambient temperature, a further addition of cholesteryl chloroformate (2.2 g) and of DIPEA (1.1 ml) is carried out and stirring is continued overnight. The medium diluted with dichloromethane is washed with 1 N HCl and then dried over Na₂SO₄, filtered and concentrated under vacuum to give an oil. After purification by column flash chromatography (dichloromethane/ethyl acetate), a solid is obtained.

Yield: 12.2 g (70%)

¹H NMR (CDCl₃, ppm): 0.67 (3H); 0.80-1.70 (29H); 1.75-2.10 (4H); 2.20-2.40 (2H); 2.75-3.15 (6H); 3.70 (9H); 3.75 (9H); 4.30-4.55 (6H); 4.55-4.65 (3H); 5.37 (1H); 5.52 (1H); 5.98 (3H).

### Hydrophobized Anionic Molecule AB3

2 N sodium hydroxide (48 ml) is added to a solution of molecule 12 (15 g; 13.7 mmol) in THF (85 ml), methanol (15 ml) and water (38 ml), and the mixture is stirred for 2 h at ambient temperature. The organic solvents are evaporated off and the medium is acidified by adding 2 N HCl. The precipitate obtained is washed with water, taken up in water, and the solution of hydrophobized anionic molecule AB3 in acid form is lyophilized.

Yield: 10.6 g (76%)

¹H NMR (DMSO-d₆, ppm): 0.67 (3H); 0.80-1.70 (29H); 1.70-2.10 (4H); 2.15-2.35 (2H); 2.50-2.75 (6H); 4.20-4.40 (9H); 5.35 (1H); 6.85-7.05 (1H); 7.53 (3H); 12.60 (6H).

LC/MS (ESI): 1028.8 ([M+NH₄]⁺); ([M+NH₄]⁺ calculated: 1029.1).

The hydrophobized anionic molecule AB3 in acid form is dissolved in water and the solution is neutralized by gradually adding 10 N sodium hydroxide to give an aqueous solution of hydrophobized anionic molecule AB3 which is then lyophilized.

¹H NMR (D₂O, ppm): 0.50-2.75 (44H); 4.15-4.60 (9H); 5.47 (1H).

### Example AB4

### Hydrophobized Anionic Molecule AB4

### Molecule 13: L-glutamic Acid 1,5-dimethyl ester isocyanate

Via a process similar to that used to prepare molecule 9, using the hydrochloride salt of L-glutamic acid 1,5-dimethyl ester (Sigma-Aldrich), molecule 13 is obtained in the form of a colorless oil.

Yield: 14.8 g (77%)

¹H NMR (CDCl₃, ppm): 1.94-2.20 (2H); 2.46 (2H); 3.60 (3H); 3.82 (3H); 4.15 (1H).

### Molecule 14: Product Obtained by Reaction Between Molecule 3 and Molecule 13

A suspension of molecule 3 (6 g; 27.1 mmol) in toluene (120 ml) is treated successively with DABCO (1.2 g; 10.84 mmol) and molecule 13 (18.3 g; 97.6 mmol). The mixture is added at 90° C. for 45 min and then concentrated under vacuum. The residue is purified by DCVC (Dry Column Vacuum Chromatography) (dichloromethane/ethyl acetate) to obtain a very pale yellow oil.

Yield: 21.2 g (quantitative)

¹H NMR (CDCl₃, ppm): 1.41 (9H); 1.80-2.05 (3H); 2.10-2.30 (3H); 2.40-2.50 (6H); 3.67 (9H); 3.75 (9H); 4.20-4.65 (9H); 5.25-5.60 (1.5H); 6.02 (2.5H).

LC/MS (ESI): 842.5 ([M+NH₄]⁺); ([M+NH₄]⁺ calculated: 842.8).

### Molecule 15: Hydrochloride Salt of the Product Obtained by Deprotection of the Boc Group of Molecule 14

Via a process similar to that used to prepare molecule 5, molecule 15 is obtained in the form of a colorless oil which solidifies over time.

Yield: 3.2 g (88%)

¹H NMR (DMSO-d₆, ppm): 1.70-2.10 (6H); 2.25-2.40 (6H); 3.50-3.75 (18H); 3.90-4.30 (10H); 7.50 (0.5H); 7.70 (2.5H); 8.70 (3H).

LC/MS (ESI): 725.6 ([M+NH₄]⁺); ([M+NH₄]⁺ calculated: 725.8).

### Molecule 16: Product Obtained by Reaction Between Molecule 15 and Cholesteryl Chloroformate

Via a process similar to that used to prepare molecule 12, molecule 16 is obtained in the form of a white solid.

Yield: 3.1 g (65%)

¹H NMR (DMSO-d₆, ppm): 0.50-2.45 (54H); 3.50-3.70 (18H); 4.00-4.35 (10H); 5.33 (1H); 6.90-7.10 (1H); 7.37 (0.5H); 7.70 (2.5H).

### Hydrophobized Anionic Molecule AB4

Via a process similar to that used to prepare the hydrophobized anionic molecule AB3, the hydrophobized anionic molecule AB4 in acid form is obtained, and taken up in water, and the solution is lyophilized.

Yield: 1.77 g (68%)

¹H NMR (DMSO-d₆, ppm): 0.50-2.30 (54H); 3.95-4.35 (10H); 5.34 (1H); 6.80-7.20 (1H); 7.54 (3H); 12.38 (6H).

LC/ms (ESI): 1070.9 ([M+NH₄]⁺); ([M+NH₄]⁺ calculated: 1071.15).

The hydrophobized anionic molecule AB4 in acid form is dissolved in water and the solution is neutralized by gradually adding 10 N sodium hydroxide to give an aqueous solution of hydrophobized anionic molecule AB4 which is then lyophilized.

¹H NMR (D₂O, ppm) 0.50-2.30 (54H); 3.75-4.75 (10H); 5.47 (1H).

### Example AC1

### Hydrophobized Anionic Molecule AC1

### Molecule 17: Carbamic Acid N-(2-aminoethyl)-1,1-dimethyl Ester

Under argon, a solution of di-tert-butyl dicarbonate (16.4 g; 75 mmol, Alfa Aesar) in dichloromethane (1.275 l), placed in a reservoir provided with a bubbler and an air inlet with a CaCl₂ trap, is added, over the course of 36 hours, to a solution of ethylenediamine (30 ml; 450 mmol, Sigma Aldrich) in dichloromethane (0.22 l). Once the addition has finished, the mixture is concentrated under vacuum. The residual oil obtained is taken up in a solution of Na₂CO₃ (2 M) which is extracted 3 times with dichloromethane. The organic phase is dried over Na₂SO₄ and concentrated under vacuum. The product obtained is distilled under vacuum to give a colourless oil.

Yield: 10.1 g (84%)

¹H NMR (CDCl₃, ppm): 1.00-1.23 (2H); 1.44 (9H); 2.78 (2H); 3.15 (2H); 4.8-5.4 (1H).

### Molecule 18: Product Obtained by Reaction Between D-gluconolactone and Molecule 17

A solution of molecule 17 in methanol (0.25 l) is added to a solution of D-gluconolactone (22.2 g; 125 mmol, Sigma Aldrich) in methanol (1 l) at 55° C. The medium is stirred at 55° C. for 2 hours and then left at ambient temperature overnight. After concentration under vacuum, the white solid obtained is taken up in diethyl ether, isolated by filtration and then dried under vacuum.

Yield: 32.7 g (77%)

¹H NMR (DMSO-d₆, ppm): 1.38 (9H); 3.00 (2H); 3.16 (2H); 3.25-3.65 (4H); 3.92 (1H); 4.08 (1H); 4.25-4.65 (4H); 5.36 (1H); 6.81 (1H); 7.75 (1H).

LC/MS (ESI): 339.5 ([M+H]⁺); ([M+H]⁺ calculated: 339.3).

### Molecule 19: Product Obtained by Reaction Between Molecule 18 and Molecule 9

Under argon, a mixture a molecule 18 (10.5 g; 31 mmol) and molecule 9 (42.1 g; 225 mmol) in pyridine is brought to 50° C. and heated a 50° C. overnight. After concentration under vacuum, the residue is taken up in ethyl acetate end washed 3 time with a 1N aqueous HCl solution. After drying over Na₂SO₄, the organic phase is filtered and concentrated to give a beige/orange solid after drying.

Yield: quantitative

LC/MS (ESI): 1291.5 ([M+NH₄]⁺); ([M+NH₄]⁺ calculated: 1291.1).

### Molecule 20: Hydrochloride Salt of the Product Obtained by Deprotection of the Boc Group of Molecule 19

A solution of molecule 19 (39.5 g; 31 mmol) in dichloromethane (155 ml) under argon is treated a 0° C. with a 4N solution a HCl in dioxane (38.8 ml). After stirring at 0° C. overnight, a further addition of 4N HCl in dioxane (15.5 ml) is carried out and the mixture is stirred at 0° C. for 1 hour. After concentration under vacuum, the residue is taken up in ethyl acetate and washed with water. The aqueous phase is then extracted twice with ethyl acetate and is then concentrated under vacuum with coevaporation with toluene. Dichloromethane is added to the oily residue and the organic phase obtained is dried over Na₂SO₄, filtered and concentrated to give viscous orange oil.

Yield: 33 g (88%)

¹H NMR (DMSO-d₆, ppm): 2.55-2.80 (12H); 3.25-3.50 (4H); 3.60-3.70 (30H); 4.00-4.25 (2H); 4.30-4.55 (5H); 4.92 (1H); 5.03 (1H); 5.17 (1H); 5.34 (1H); 7.50-8.00 (7H); 8.37 (1H).

LC/MS (ESI): 1175.3 ([M-Cl]); ([M-Cl] calculated: 1175).

### Molecule 21: Product Obtained by Reaction Between Molecule 20 and Cholesteryl Chloroformate

A solution of molecule 20 (33 g; 27.3 mmol) in dichloromethane (225 ml) at 0° C. is treated with triethylamine (7.9 ml). After 20 min, a solution of cholesteryl chloroformate (10.2 g; 22.7 mmol, Alfa Aesar) in dichloromethane (60 ml) is added dropwise over the course of 50 min and the mixture is then stirred from 0° C. to ambient temperature for 90 min. The medium is transferred into a separating funnel and washed twice with a 1N HCl solution. After drying over Na₂SO₄, the organic phase is filtered and concentrated to give an orange solid which is purified by flash chromatography (dichloromethane/acetone) to give molecule 21 in the form of a white solid.

Yield: 16.2 g (45%)

¹H NMR (DMSO-d₆, ppm): 0.65 (3H); 0.75-1.60 (33H); 1.75-2.00 (5H); 2.25 (2H); 2.55-2.75 (10H); 2.90-3.25 (4H); 3.60-3.70 (30H); 4.00-4.50 (8H); 4.80-5.40 (5H); 6.87 (1H); 7.25-7.80 (5H); 8.03 (1H).

LC/MS (ESI): 1604.4 ([M+NHₐ]⁺); ([M+NH₄]⁺ calculated: 1603.6).

### Hydrophobized Anionic Molecule AC1

A 2N NaOH solution (24.8 ml) is added dropwise, over the course of 20 min, to a solution of molecule 21 (7.5 g; 4.73 mmol) in a THF/water mixture (37.5 ml/9.5 ml) at 2° C. After addition of the sodium hydroxide solution, a precipitate forms. The medium is stirred for a few minutes while cold and then the ice bath is removed. After stirring for 30 min, the mixture is filtered and the solid is washed with a THF/water mixture (50/50 vol.), taken up in water and then lyophilized. The solid is taken up in water and acidified using an organic acid supported on resin (MP-TsOH) washed beforehand with water. The solution is filtered on a sintered glass filter (P3) and the filtrate is lyophilized to give a white solid of hydrophobized anionic molecule AC1 in acid form.

Yield: 4 g (58%)

¹H NMR (DMSO-d₆; ppm: 0.50-160 (36H); 1.75-2.00 (5H); 2.25 (2H); 2.55-2.75 (10H); 2.90-3.25 (4H); 4.00-4.50 (8H); 4.90-5.40 (5H); 6.75-7.50 (6H); 7.98 (1H); 12.60 (10H).

LC/ms (ESI): 1463.7 ([M+NH₄]⁺); ([M+NH₄]⁺ calculated: 1463.4).

The hydrophobized anionic molecule AC1 in acid form is dissolved in water and the solution is neutralized by gradually adding 10N sodium hydroxide to give an aqueous solution of hydrophobized anionic molecule AC1 which is then lyophilized.

¹H NMR (D₂O, ppm): 0.50-3.00 (53H); 3.15-3.50 (4H); 4.00-4.50 (8H); 5.00-5.60 (5H).

### Part B

### Demonstration of the Properties of the Compositions as Claimed in the Invention

### Example B1

### 100 IU/ml Solution of Fast-Acting Insulin Analog (NovoLog®)

This solution is a commercial solution of insulin aspart sold by the company NOVO NORDISK under the name NovoLog® in the United States of America and Novorapid® in Europe. This product is a fast-acting insulin analog.

### Example B2

### 100 IU/ml Solution of Fast-Acting Insulin Analog (Humalog®)

This solution is a commercial solution of insulin lispro sold by the company ELI LILLY under the name Humalog®. This product is a fast-acting insulin analog.

### Example B3

### 100 IU/ml Solution of Fast-Acting Insulin Analog (Adipra®)

This solution is a commercial solution of insulin glulisine sold by the company SANOFI-AVENTIS under the name Apidra®. This product is a fast-acting insulin analog.

### Example B4

### 100 IU/ml Solution of Slow-Acting Insulin Analog (Lantus®)

This solution is a commercial solution of insulin glargine sold by the company SANOFI-AVENTIS under the name Lantus®. This product is a slow-acting insulin analog.

### Example B5

### 100 IU/ml Solution of Human Insulin (ActRapid®)

This solution is a commercial solution of human insulin from NOVO NORDISK sold under the name ActRapid®. This product is a human insulin.

### Example B6

### Solubilization of Insulin Glargine at 100 IU/ml and at pH 7 Using a Hydrophobized Anionic Molecule at the Concentration of 10 mg/ml

20 mg of a hydrophobized anionic molecule chosen from those described in table 1 are accurately weighed out. This lyophilisate is taken up with 2 ml of the insulin glargine solution of example 84 in order to obtain a solution of which the hydrophobized anionic molecule concentration is equal to 10 mg/ml as described in table 2. After mechanical stirring on rollers at ambient temperature, the solution becomes clear. The pH of this solution is below 7. The pH is adjusted to 7 with a 0.1 N sodium hydroxide solution. This clear solution is filtered through a membrane (0.22 μm) and is then placed at +4° C.

The solubilization test according to the protocol above was carried out with various hydrophobized anionic molecules. These solutions are referenced in table 2.

TABLE 2
Solutions according to example B6 with the hydrophobized
anionic molecules having a concentration of 10 mg/ml

| | | Concentration of |
|---|---|---|
| Solution | Hydrophobized | hydrophobized anionic |
| example B6 | anionic molecule | molecule |
| B6(a) | AA1 | 10 mg/ml |
| B6(b) | AA2 | 10 mg/ml |
| B6(c) | AA3 | 10 mg/ml |
| B6(d) | AA4 | 10 mg/ml |
| B6(e) | AA5 | 10 mg/ml |
| B6(f) | AB1 | 10 mg/ml |
| B6(g) | AB2 | 10 mg/ml |
| B6(h) | AB3 | 10 mg/ml |
| B6(i) | AB4 | 10 mg/ml |
| B6(j) | AC1 | 10 mg/ml |
| B6(k) | AA6 | 10 mg/ml |
| B6(l) | AA7 | 10 mg/ml |
| B6(m) | AA8 | 10 mg/ml |
| B6(n) | AA9 | 10 mg/ml |

Generalyzation:

Clear solutions of insulin glargine at 100 IU/ml and at pH 7 were also obtained with hydrophobized anionic molecule concentrations of 20, 40 or 60 mg/ml according to the same protocol as that described in example B6. Thus, a weight of a lyophilized hydrophobized anionic molecule among those described in table 1 is accurately weighed out. This lyophilisate is taken up with 2 ml of the insulin glargine solution of example B4 so as to obtain a solution of which the hydrophobized anionic molecule concentration is 20, 40 or 60 mg/ml as described in table 3. After mechanical stirring on rollers at ambient temperature, the solution becomes clear. The pH of this solution is below 7. The pH is then adjusted to 7 with a 0.1 N sodium hydroxide solution. This clear final solution is filtered through a membrane (0.22 μm) and is then placed at +4° C.

TABLE 3
Preparation of a solution of insulin glargine at 100
IU/ml and at pH 7 using a hydrophobized anionic molecule
at the concentration of 20, 40 or 60 mg/ml

| Final concentration | | Volume of the insulin |
|---|---|---|
| of hydrophobized | Weight of hydrophobized | glargine solution of |
| anionic molecule | anionic molecule weighed | example B4 added |
| (mg/ml) | out (mg) | (ml) |
| 20 | 40 | 2 |
| 40 | 80 | 2 |
| 60 | 120 | 2 |

### Example B7

### Preparation of a Hydrophobized Anionic Molecule AA1/Insulin Glargine/Insulin Glulisine Composition with a 75/25 Insulin Glargine/Insulin Glulisine Ratio at pH 7

0.25 ml of the insulin glulisine solution of example B3 is added to 0.75 ml of the solution of hydrophobized anionic molecule AA1/insulin glargine prepared according to the protocol described in example B6(a), so as to form 1 ml of a composition at pH 7. The composition is clear, attesting to the good solubility of the insulin glargine and of the insulin glulisine under these formulation conditions. This clear solution is filtered through a 0.22 μm filter and then placed at +4° C.

### Example B8

### Preparation of a Hydrophobized Anionic Molecule AA1/Insulin Glargine/Insulin Lispro Composition with a 75/25 Insulin Glargine/Insulin Lispro Ratio at pH 7

0.25 ml of the insulin lispro solution of example B2 is added to 0.75 ml of the solution of hydrophobized anionic molecule AA1/insulin glargine prepared according to the protocol described in example B6(a), so as to form 1 ml of a composition at pH 7. The composition is clear, attesting to the good solubility of the insulin glargine and of the insulin lispro under these formulation conditions. This clear solution is filtered through a 0.22 μm filter and then placed at +4° C.

### Example B9

### Preparation of a Hydrophobized Anionic Molecule AA1/Insulin Glargine/Insulin Aspart Composition with a 75/25 Insulin Glargine/Insulin Aspart Ratio at pH 7

0.25 ml of the insulin aspart solution of example B1 is added to 0.75 ml of the solution of hydrophobized anionic molecule AA1/insulin glargine prepared in example B6(a), so as to form 1 ml of a composition at pH 7. The composition is clear, attesting to the good solubility of the insulin glargine and of the insulin aspart under these formulation conditions. This clear solution is filtered through a 0.22 μm filter and then placed at +4° C.

### Example B10

### Preparation of a Hydrophobized Anionic Molecule AA1/Insulin Glargine/Human Insulin Composition with a 75/25 Insulin Glargine/Human Insulin Ratio at pH 7

0.25 ml of the human insulin solution of example B5 is added to 0.75 ml of the solution of hydrophobized anionic molecule AA1/insulin glargine prepared in example B6(a), so as to form 1 ml of a composition at pH 7. The composition is clear, attesting to the good solubility of the insulin glargine and of the human insulin under these formulation conditions. This clear solution is filtered through a 0.22 μm filter and than placed at +4° C.

### Example 811

### Preparation of a Hydrophobized Anionic Molecule AA1/Insulin Glargine/Insulin Lispro Composition with a 60/40 Insulin Glargine/Insulin Lispro Ratio at pH 7

0.4 ml of the insulin lispro solution of example B2 is added to 0.6 ml of the solution of hydrophobized anionic molecule AA1/insulin glargine prepared in example 86(a), so as to form 1 ml of a composition at pH 7. The composition is clear, attesting to the good solubility of the insulin glargine and of the insulin lispro under these formulation conditions. This clear solution is filtered through a 0.22 μm filter and then placed at +4° C.

### Example B12

### Preparation of a Hydrophobized Anionic Molecule AA1/Insulin Glargine/Insulin Lispro Composition with a 40/60 Insulin Glargine/Insulin Lispro Ratio at pH 7

0.6 ml of the insulin lispro solution of example B2 is added to 0.4 ml of the solution of hydrophobized anionic molecule AA1/insulin glargine prepared in example B6(a), so as to form 1 ml of a composition at pH 7. The composition is clear, attesting to the good solubility of the insulin glargine and of the insulin lispro under these formulation conditions. This clear solution is filtered through a 0.22 μm filter and then placed at +4° C.

### Example B13

### Insulin Glargine Precipitation

1 ml of the insulin glargine solution of example B4 is added to 2 ml of a solution of PBS (phosphate buffered saline) containing 20 mg/ml of BSA (bovine serum albumin). The PBS/BSA mixture simulates the composition of the subcutaneous medium. A precipitate appears, which is in good agreement with the mechanism via which insulin glargine works (precipitation upon injection due to increased pH).

Centrifugation at 4000 rpm is carried out in order to separate the precipitate from the supernatant. The insulin glargine is then assayed in the supernatant by reverse-phase liquid chromatography (RP-HPLC). The result is that the insulin glargine is predominantly in a precipitated form.

### Example B14

### Precipitation of an Anionic Molecule AA1/Insulin Glargine Composition

1 ml of hydrophobized anionic molecule AA1/insulin glargine solution prepared in example B6(a) is added to 2 ml of a solution of PBS containing 20 mg/ml of BSA. The PBS/BSA mixture simulates the composition of the subcutaneous medium. A precipitate appears.

Centrifugation at 4000 rpm is carried out in order to separate the precipitate from the supernatant. The insulin glargine is then assayed in the supernatant by RP-HPLC. The result is that the insulin glargine is predominantly in a precipitated form.

Solubilization and precipitation tests identical to those described in examples B6 and B14 were carried out with other hydrophobized anionic molecules at the same concentration of 10 mg/ml of hydrophobized anionic molecule for 100 IU/ml of insulin glargine solution. The result is that, for all the compositions B6(b) to B6(n), the insulin glargine is predominantly in a precipitated form after the addition of 1 ml of the composition to 2 ml of a solution of PBS containing 20 mg/ml of BSA. The results are summarized in table 4.

TABLE 4
Tests for solubilization and precipitation of a hydrophobized
anionic molecule/insulin glargine composition

| Hydrophobized anionic | Insulin glargine | Insulin glargine |
|---|---|---|
| molecule (10 mg/ml) | solubilization | precipitation |
| AA1 | Yes | Yes |
| AA2 | Yes | Yes |
| AA3 | Yes | Yes |
| AA4 | Yes | Yes |
| AA5 | Yes | Yes |
| AA6 | Yes | Yes |
| AA7 | Yes | Yes |
| AA8 | Yes | Yes |
| AA9 | Yes | Yes |
| AB1 | Yes | Yes |
| AB2 | Yes | Yes |
| AB3 | Yes | Yes |
| AB4 | Yes | Yes |
| AC1 | Yes | Yes |

### Example B15

### Precipitation of a Hydrophobized Anionic Molecule AA1/Insulin Glargine/Insulin Lispro Composition with a 75/25 Insulin Glargine/Insulin Lispro Ratio at pH 7

1 ml of the hydrophobized anionic molecule AA1/insulin glargine/insulin lispro 75/25 composition prepared according to the protocol of example 88 is added to 2 ml of a solution of PBS containing 20 mg/ml of BSA. The PBS/BSA mixture simulates the composition of the subcutaneous medium. A precipitate appears.

Centrifugation at 4000 rpm is carried out in order to separate the precipitate from the supernatant. The insulin glargine is then assayed in the supernatant by RP-HPLC. The result is that the insulin glargine is predominantly in a precipitated form.

### Example B16

### Precipitation of Various Compositions while Varying the Nature of the Hydrophobized Anionic Molecule

Other insulin glargine precipitation tests under the same conditions as those of example B15 were carried out in the presence of other hydrophobized anionic molecules.

The results are collated in the following table 5, and it is observed that the solubilization and also the precipitation of the insulin glargine are preserved.

TABLE 5
Tests for solubilization and precipitation of a hydrophobized anionic
molecule/insulin glargine/insulin lispro 75/25 composition at pH 7

| | Solubilization of | |
|---|---|---|
| Hydrophobized | insulin glargine/ | Insulin glargine |
| anionic molecule | insulin lispro 75/25 | precipitation |
| AA1 | Yes | Yes |
| AA2 | Yes | Yes |
| AA3 | Yes | Yes |
| AA4 | Yes | Yes |
| AA5 | Yes | Yes |
| AA6 | Yes | Yes |
| AA7 | Yes | Yes |
| AA8 | Yes | Yes |
| AA9 | Yes | Yes |
| AB1 | Yes | Yes |
| AB2 | Yes | Yes |
| AB3 | Yes | Yes |
| AB4 | Yes | Yes |
| AC1 | Yes | Yes |

### Example B17

### Precipitation of Various Compositions while Varying the Nature of the Prandial Insulin

Compositions are prepared by mixing 0.75 ml of the hydrophobized anionic molecule AA1/insulin glargine solution prepared according to the protocol of example B6(a) with 0.25 ml of a prandial insulin, so as to form 1 ml of hydrophobized anionic molecule AA1/insulin glargine/prandial insulin composition (containing 7.5 mg/ml of hydrophobized anionic molecule AA1, 75 IU/ml of insulin glargine and 25 IU/ml of prandial insulin).

This composition is added to 2 ml of PBS containing 20 mg/ml of BSA. The PBS/BSA mixture simulates the composition of the subcutaneous medium. A precipitate appears. Centrifugation at 4000 rpm is carried out in order to separate the precipitate from the supernatant. The insulin glargine is then assayed in the supernatant by RP-HPLC. The result is that the insulin glargine is predominantly in a precipitated form. In the presence of the 4 prandial insulins tested, the insulin glargine precipitates from the PBS/BSA mixture. The results are collated in table 6.

TABLE 6
Tests for solubilization and precipitation of a hydrophobized anionic
molecule AA1/insulin glargine/prandial insulin 75/25 composition

| | Solubilization of | |
|---|---|---|
| | insulin glargine/ | Insulin glargine |
| Nature of the prandial insulin | prandial insulin 75/25 | precipitation |
| Insulin glulisine (Apidra ®) | Yes | Yes |
| Insulin aspart (NovoLog ®) | Yes | Yes |
| Insulin lispro (Humalog ®) | Yes | Yes |
| Human insulin (ActRapid ®) | Yes | Yes |

### Example B18

### Preparation of a Concentrated Solution of Slow-Acting Insulin Analog (Insulin Glargine)

A commercial solution of insulin glargine sold by the company SANOFI-AVENTIS under the name Lantus®, of example B4, is concentrated by ultrafiltration on a 3 kDa regenerated cellulose membrane (Amicon® Ultra-15 sold by the company Millipore). At the end of this ultrafiltration step, the insulin glargine concentration is assayed in the retentate by reverse-phase liquid chromatography (RP-HPLC). The final concentration of insulin glargine is then adjusted by adding commercial insulin glargine solution at 100 IU/ml so as to obtain the desired final concentration. This progress makes it possible to obtain concentrated solutions a insulin glargine, denoted C_{insulin glargine} at various concentrations greater than 100 IU/ml, such as C_{insulin glargine}=200, 250, 300 and 333 IU/ml. The concentration solutions are filtered through a 0.22 μm filter and then stored at +4° C.

### Example B19

### Dialysis of a Commercial Solution of Fast-Acting Insulin Analog (Insulin Lispro)

A commercial solution of insulin lispro sold by the company ELI LILLY under the name Humalog® is dialyzed by ultrafiltration on a 3 kDa regenerated cellulose membrane (Amicon® Ultra-15 sold by the company Millipore). The dialysis is carried out in a 1 mM phosphate buffer at pH 7. At the end of this dialysis step, the concentration C_{insulin lispro dialyzed} in the retentate is determined by reverse-phase liquid chromatography (RP-HPCL). The solution dialyzed is stored in a freezer at −80° C.

### Example B20

### Lyophilization of a Solution of Fast-Acting Insulin Analog (Insulin Lispro) in its Commercial Form

A volume V_{Humalog} of a solution of fast-acting insulin lispro at a concentration at 100 IU/ml in its commercial form is placed in a Iyogard® tray sterilized beforehand in an autoclave. The Iyogard® tray is placed in a freezer at −80° C. for approximately 1 h and then lyophilzation is carried out with the parameters of temperature 20° C. and pressure 0.31 mbar.

The resulting sterile lyophilisate is stored at ambient temperature.

### Example B21

### Lyophilization of a Commercial Solution of Fast-Acting Insulin Analog (Insulin Lispro) which has been Dialyzed

A volume V_{Humalog dialyzed} of a solution of fast-acting insulin lispro obtained according to example B19, at a concentration C_{lispro dialyzed}, is placed in a Iyogard® tray sterilized beforehand in an autoclave. The Iyogard® tray is placed in a freezer at −80° C. for approximately 1 h and then lyophilization is carried out with the parameters of temperature 20° C. and pressure 0.31 mbar.

The resulting sterile lyophilisate is stored at ambient temperature.

### Example 822

### Preparation of a Concentrated Hydrophobized Anionic Molecule/Insulin Glargine Composition at pH 7 Using the Hydrophobized Anionic Molecule According to a Process Using Insulin Glargine in Liquid Form (in Solution) and a Hydrophobized Anionic Molecule in Solid Form (Lyophilized)

A weight w_{hydrophobized anionic molecule} of hydrophobized anionic molecule is accurately weighed out. This lyophilisate is taken up with a volume V_{insulin glargine} of a concentrated solution of insulin glargine prepared according to example B18 so as to obtain a composition having a hydrophobized anionic molecule concentration C_{hydrophobized anionic molecule} (mg/ml)=w_{hydrophobized anionic molecule}/V_{insulin glargine} and an insulin glargine concentration C_{insulin glargine} (IU/ml). The solution is opalescent. The pH of this solution is approximately 6.3. The pH is adjusted to 7 by adding concentrated NaOH and then the solution is placed statically in an incubator at 37° C. for approximately 1 hour until complete solubilization is obtained. A volume V_{hydrophobized anionic molecule/insulin glargine} of this visually clear solution is placed at +4° C.

### Example B23

### Preparation of a Hydrophobized Anionic Molecule/Insulin Glargine Composition at pH 7 Using a Hydrophobized Anionic Molecule, According to a Process Using Insulin Glargine in Liquid Form (in Solution) and a Hydrophobized Anionic Molecule in Liquid Form (in Solution)

Concentrated solutions of m-cresol, glycerol and Tween® 20 are added to a stock solution of hydrophobized anionic molecule at pH 7 which has a concentration C_{stock hydrophobized anionic molecule}, so as to obtain a solution of hydrophobized anionic molecule having a concentration C_{stock hydrophobized anionic molecule/excipients} (mg/ml) in the presence of these excipients at contents equivalent to those described in the commercial solution Lantus® in a 10 ml bottle.

In a sterile pot, a volume V_{Lantus} of a commercial solution of slow-acting insulin glargine sold under the name Lantus® at a concentration of 100 IU/ml is added to a volume V_{stock hydrophobized anionic molecule/excipients} of a solution of hydrophobized anionic molecule at the concentration C_{stock hydrophobized anionic molecule/excipients} (mg/ml). A cloudiness appears. The pH is adjusted to 7 by adding concentrated NaOH and the solution is placed statically in an incubator at 37° C. for approximately 1 hour until complete solubilization is obtained. This visually clear solution is placed at +4° C.

### Example B24

### Preparation of a Concentrated Hydrophobized Anionic Molecule/Insulin Glargine Composition at pH=7 Using a Hydrophobized Anionic Molecule, According to a Process for Concentrating a Dilute Composition

A dilute hydrophobized anionic molecule/insulin glargine composition at pH 7 described in example B23 is concentrated by ultrafiltration on a 3 kDa regenerated cellulose membrane (Amicon® Ultra-15 sold by the company Millipore). At the end of this ultrafiltration step, the retentate is clear and the insulin glargine concentration in the composition is determined by RP-HPLC. If necessary, the insulin glargine concentration in the composition is then adjusted to the desired value by dilution in a solution excipients m-cresol/glycerol/Tween®20 having, for each entity, a concentration equivalent to that described in the commercial solution Lantus® (in a 10 ml bottle). This solution at pH 7, which is visually clear, and which has an insulin glargine concentration C_{insulin glargine} (IU/ml) and a hydrophobized anionic molecule concentration C_{hydrophilized anionic molecule} (mg/ml), is placed at +4° C.

### Example B25

### Preparation of a Hydrophobized Anionic Molecule/Insulin Glargine/Insulin Lispro Composition at pH 7, from a Lyophilisate of a Fast-Acting Insulin Lispro to its Commercial form (Humalog®)

A volume V_{hydrophilized anionic molecule/insulin glargine} of hydrophobized anionic molecule/insulin glargine solution, pH 7, having an insulin glargine concentration C_{insulin glargine} (IU/ml) and a hydrophobized anionic molecule concentration C_{hydrophobized anionic molecule} (mg/ml) prepared according to example B22 is added to a lyophilisate of insulin lispro obtained by lyophilization of a volume V_{insulin lispro} of which the preparation is described in example B20, such that the ratio V_{hydrophobized anionic molecule/insulin glargine}/V_{insulin lispro}=100/C_{insulin lispro} where C_{insulin lispro} is the concentration of insulin lispro (IU/ml) targeted in the composition. The solution is clear. The zinc content of the formulation is adjusted to the desired concentration C_{zinc} (μM) by adding a concentrated solution of zinc chloride. The final pH is adjusted to 7 by adding concentrated NaOH or HCl.

The formulation is clear, attesting to the good solubility of insulin glargine and insulin lispro tinder these formulation conditions. This solution is filtered through a 0.22 μm filter and placed at +4° C.

### Example B26

### Preparation of a Hydrophobized Anionic Molecule/Insulin Glargine/Insulin Lispro Composition at pH 7, from a Lyophilisate of a Fast-Acting Insulin Lispro Obtained by Dialysis of a Commercial Solution (Humalog®)

A volume V_{hydrophobized anionic molecule/insulin glargine} of hydrophobized anionic molecule/insulin glargine solution, pH 7, having an insulin glargine concentration C_{insulin glargine} (IU/ml) and a hydrophobized anionic molecule concentration C_{hydrophobized anionic molecule} (mg/ml) prepared according to example B22 is added to a lyophilisate of insulin lispro obtained by lyophilzation of a volume V_{insulin lispro dialyzed} of which the preparation is described in example B21, such that the ratio V_{hydrophobized anionic molecule/insulin glargine}/V_{insulin lispro dialyzed}=C_{insulin lispro dialyzed}/C_{insulin lispro} where C_{insulin lispro dialyzed} is the concentration of insulin lispro (IU/ml) obtained at the end of the dialysis of the commercial solution, the step, described in example B19, and C_{insulin lispro} is the concentration of insulin lispro (IU/ml) targeted in the composition. The solution is clear. The zinc content of the formulation is adjusted to the desired concentration C_{zinc} (μM) by adding a concentrated solution of zinc chloride. The final pH is adjusted to 7 by adding concentrated NaOH or HCl.

The formulation is clear, attesting to the good solubility of the insulins glargine and lispro under these formulation conditions. This solution is filtered through a 0.22 μm filter and placed at +4° C.

### Example B27

### Preparation of a Hydrophobized Anionic Molecule AB1/Insulin Glargine/Insulin Lispro Composition at pH 7 Having an Insulin Glargine Concentration of 200 IU/ml and an Insulin Lispro Concentration of 66 IU/ml (Percentage Proportion of Insulin: Insulin Glargine/Insulin Lispro 75/25)

A concentrated insulin glargine solution at 200 IU/ml is prepared according to example B18. A hydrophobized anionic molecule AB1 (12 mg/ml)/insulin glargine 200 IU/ml composition, pH 7, is prepared from a hydrophobized anionic molecule AB1 and according to the preparation method described in example B22. This hydrophobized anionic molecule AB1/insulin glargine 200 IU/ml composition is added to a lyophilisate of insulin lispro obtained by lyophilization of the solution of fast-acting analog derived from the dialysis of a commercial solution, according to the preparation method described in example B26. The solution is clear. The zinc content of the formulation is adjusted to the desired concentration by adding a concentrated solution of zinc chloride. The final pH is adjusted to 7 by adding concentrated NaOH or HCl.

The formulation is clear, attesting to the good solubility of the insulins glargine and lispro under these formulation conditions. This solution is filtered through a 0.22 μm filter and placed at +4° C.

This composition is described in table 11.

Hydrophobized anionic molecule/insulin glargine/insulin lispro 200/66 compositions at pH 7 were also prepared with other hydrophobized anionic molecules according to a preparation method identical to that described in example B27 with a hydrophobized anionic molecule concentration of at 25 mg/ml. These formulations are clear, attesting to the good solubility of the insulins glargine and lispro under these formulation conditions. These compositions result in the examples listed in table 7.

TABLE 7

| | | | | | Cᵢₙₛᵤₗᵢₙ |
|---|---|---|---|---|---|
| | Hydro- | Concentration of | | | _{glargine}/ |
| | phobized | hydrophobized | Cᵢₙₛᵤₗᵢₙ | Cᵢₙₛᵤₗᵢₙ | Cᵢₙₛᵤₗᵢₙ |
| Exam- | anionic | anionic molecule | _{glargine} | ₗᵢₛₚᵣₒ | ₗᵢₛₚᵣₒ |
| ple | molecule | (mg/ml) | (UI/mL) | (UI/mL) | (%/%) |
| B27 | AB1 | 12 | 200 | 66 | 75/25 |
| B28 | AA3 | 25 | 200 | 66 | 75/25 |
| B29 | AB2 | 25 | 200 | 66 | 75/25 |
| B30 | AB3 | 25 | 200 | 66 | 75/25 |
| B31 | AB4 | 25 | 200 | 66 | 75/25 |
| B32 | AC1 | 25 | 200 | 66 | 75/25 |

These compositions are described in table 11.

### Example B33

### Preparation of a Hydrophobized Anionic Molecule AB1/Insulin Glargine/Insulin Lispro Composition at pH 7 Having an Insulin Glargine Concentration of 300 IU/ml and an Insulin Lispro Concentration of 100 IU/ml (Percentage Proportion of Insulin: Insulin Glargine/Insulin Lispro 75/25)

A concentrated insulin glargine solution at 300 IU/ml is prepared according to example B18. A hydrophobized anionic molecule AB1 (17 mg/ml)/insulin glargine 300 IU/ml composition, pH 7, is prepared from the hydrophobized anionic molecule AB1 and according to the preparation method described in example B22. This hydrophobized anionic molecule AB1/insulin glargine 300 IU/ml composition is added to a lyophilisate of insulin lispro obtained by lyophilization of the solution of fast-acting analog derived from the dialysis of a commercial solution, according to the preparation method described in example B26. The solution is clear. The zinc content of the formulation is adjusted to the desired concentration by adding a concentrated solution of zinc chloride. The final pH is adjusted to 7 by adding concentrated NaOH or HCl.

The formulation is clear, attesting to the good solubility of the insulins glargine and lispro under these formulation conditions. This solution is filtered through a 0.22 μm filter and placed at +4° C. This composition is described in table 11.

Hydrophobized anionic molecule/insulin glargine/insulin lispro 300/100 compositions at pH 7 were also prepared with other hydrophobized anionic molecules according to a preparation method identical to that described in example 833 with a hydrophobized anionic molecule concentration of 40 mg/ml. These formulations are clear, attesting to the good solubility of the insulins glargine and lispro under these formulation conditions. These compositions result in the examples listed in table 8.

TABLE 8

| | | | | | Cᵢₙₛᵤₗᵢₙ |
|---|---|---|---|---|---|
| | Hydro- | Concentration of | | | _{glargine}/ |
| | phobized | hydrophobized | Cᵢₙₛᵤₗᵢₙ | Cᵢₙₛᵤₗᵢₙ | Cᵢₙₛᵤₗᵢₙ |
| Exam- | anionic | anionic molecule | _{glargine} | ₗᵢₛₚᵣₒ | ₗᵢₛₚᵣₒ |
| ple | molecule | (mg/ml) | (UI/mL) | (UI/mL) | (%/%) |
| B33 | AB1 | 17 | 300 | 100 | 75/25 |
| B34 | AA3 | 40 | 300 | 100 | 75/25 |
| B35 | AA8 | 40 | 300 | 100 | 75/25 |
| B36 | AB2 | 40 | 300 | 100 | 75/25 |
| B37 | AB3 | 40 | 300 | 100 | 75/25 |
| B38 | AB4 | 40 | 300 | 100 | 75/25 |
| B39 | AC1 | 40 | 300 | 100 | 75/25 |

These compositions are described in table 11.

### Example B40

### Preparation of a Hydrophobized Anionic Molecule AB1/Insulin Glargine/Insulin Lispro Composition at pH 7 Having an Insulin Glargine Concentration of 250 IU/ml and an Insulin Lispro Concentration of 150 IU/ml (Percentage Proportion of Insulin: Insulin Glargine/Insulin Lispro 63/37)

A concentrated insulin glargine solution at 250 IU/ml is prepared according to example B18. A hydrophobized anionic molecule AB1 (14 mg/ml)/insulin glargine 250 IU/ml composition, pH 7, is prepared from the hydrophobized anionic molecule AB1 and according to the preparation method described in example B22. This hydrophobized anionic molecule AB1/insulin glargine 250 IU/ml composition is added to a lyophilisate of insulin lispro obtained by lyophilization of the solution of fast-acting analog derived from the dialysis of a commercial solution, according to the preparation method described in example B26. The solution is clear. The zinc content of the formulation is adjusted to the desired concentration by adding a concentrated solution of zinc chloride. The final pH is adjusted to 7 by adding concentrated NaOH or HCl.

The formulation is clear, attesting to the good solubility of the insulins glargine and lispro under these formulation conditions. This solution is filtered through a 0.22 μm filter and placed at +4° C.

This composition is described in table 11.

Hydrophobized anionic molecule/insulin glargine/insulin lispro 250/150 compositions at pH 7 were also prepared according to a preparation method identical to that described in example B40 with a hydrophobized anionic molecule concentration of 35 mg/ml. These formulations are clear, attesting to the good solubility of the insulins glargine and lispro under these formulation conditions. These compositions result in the examples listed in table 9.

TABLE 9

| | | | | | Cᵢₙₛᵤₗᵢₙ |
|---|---|---|---|---|---|
| | Hydro- | Concentration of | | | _{glargine}/ |
| | phobized | hydrophobized | Cᵢₙₛᵤₗᵢₙ | Cᵢₙₛᵤₗᵢₙ | Cᵢₙₛᵤₗᵢₙ |
| Exam- | anionic | anionic molecule | _{glargine} | ₗᵢₛₚᵣₒ | ₗᵢₛₚᵣₒ |
| ple | molecule | (mg/ml) | (UI/mL) | (UI/mL) | (%/%) |
| B41 | AA3 | 35 | 250 | 150 | 63/37 |
| B42 | AB2 | 35 | 250 | 150 | 63/37 |
| B43 | AB3 | 35 | 250 | 150 | 63/37 |
| B44 | AB4 | 35 | 250 | 150 | 75/25 |
| B45 | AC1 | 35 | 250 | 150 | 75/25 |

These compositions are described in table 11.

### Example B46

### Preparation of a Hydrophobized Anionic Molecule AA4/Insulin Glargine/Insulin Lispro Composition at pH 7 Having an Insulin Glargine Concentration of 300 IU/ml and an Insulin Lispro Concentration of 100 IU/ml (Percentage Proportion of Insulin: Insulin Glargine/Insulin Lispro 75/25)

A hydrophobized anionic molecule AA4 (12 mg/ml)/insulin glargine 300 IU/ml composition, pH 7, is prepared from a hydrophobized anionic molecule AA4 and according to the preparation method described in example B23 and B24. This hydrophobized anionic molecule AA4/insulin glargine 300 IU/ml composition is added to a lyophilisate of insulin lispro obtained by lyophilization of the solution of fast-acting analog derived from the dialysis of a commercial solution, according to the preparation method described in example B26. The solution is clear. The zinc content of the formulation is adjusted to the desired concentration by adding a concentrated solution of zinc chloride. The final pH is adjusted to 7 by adding concentrated NaOH or HCl.

The formulation is clear, attesting to the good solubility of the insulins glargine and lispro under these formulation conditions. This solution is filtered through a 0.22 μm filter and placed at +4° C.

This composition is described in table 11.

Hydrophobized anionic molecule/insulin glargine/insulin lispro 300/100 compositions at pH 7 were also prepared with other hydrophobized anionic molecules according to a preparation method identical to that described in example B46 with a hydrophobized anionic molecule concentration of at most 40 mg/ml. These formulations are clear, attesting to the good solubility of the insulins glargine and lispro under these formulation conditions. These compositions result in the examples listed in table 10.

TABLE 10

| | | | | | Cᵢₙₛᵤₗᵢₙ |
|---|---|---|---|---|---|
| | Hydro- | Concentration of | | | _{glargine}/ |
| | phobized | hydrophobized | Cᵢₙₛᵤₗᵢₙ | Cᵢₙₛᵤₗᵢₙ | Cᵢₙₛᵤₗᵢₙ |
| Exam- | anionic | anionic molecule | _{glargine} | ₗᵢₛₚᵣₒ | ₗᵢₛₚᵣₒ |
| ple | molecule | (mg/ml) | (UI/mL) | (UI/mL) | (%/%) |
| B47 | AA3 | 10 | 300 | 100 | 75/25 |
| B48 | AA5 | 15 | 300 | 100 | 75/25 |
| B49 | AA6 | 40 | 300 | 100 | 75/25 |
| B50 | AA7 | 40 | 300 | 100 | 75/25 |
| B51 | AA9 | 40 | 300 | 100 | 75/25 |

These compositions are described in table 11.

### Example B52

### Precipitation of Various Hydrophobized Anionic Molecule/Insulin Glargine/Insulin Lispro Compositions at pH 7 Having Various Insulin Glargine and Insulin Lispro Concentrations

1 ml of hydrophobized anionic molecule/insulin glargine/insulin lispro composition prepared in examples 827 to B51 is added to 2 ml of a solution of PBS containing 20 mg/ml of BSA. The PBS/BSA mixture simulates the composition of the subcutaneous medium. A precipitate appears. Centrifugation at 4000 rpm is carried out in order to separate the precipitate from the supernatant. The insulin glargine is then assayed in the supernatant by RP-HPLC. The result is that the insulin glargine is predominantly in a precipitated form.

The solubilization and precipitation results are summarized in table 11.

TABLE 11
Tests for solubilization and precipitation of various hydrophobized
anionic molecule/insulin glargine/insulin lispro compositions at
pH 7 having various insulin glargine and insulin lispro concentrations
and various relative proportions of the 2 insulins

| | | Cᵢₙₛᵤₗᵢₙ | | |
|---|---|---|---|---|
| | | _{glargine}/ | Solubilization | |
| | Hydrophobized | Cᵢₙₛᵤₗᵢₙ | insulin glargine | Insulin |
| | anionic | _{glargine} | and insulin | glargine |
| Example | molecule | (%/%) | lispro at pH 7 | precipitation |
| B27 | AB1 | 75/25 | YES | YES |
| B28 | AA3 | 75/25 | YES | YES |
| B29 | AB2 | 75/25 | YES | YES |
| B30 | AB3 | 75/25 | YES | YES |
| B31 | AB4 | 75/25 | YES | YES |
| B32 | AC1 | 75/25 | YES | YES |
| B33 | AB1 | 75/25 | YES | YES |
| B34 | AA3 | 75/25 | YES | YES |
| B35 | AA8 | 75/25 | YES | YES |
| B36 | AB2 | 75/25 | YES | YES |
| B37 | AB3 | 75/25 | YES | YES |
| B38 | AB4 | 75/25 | YES | YES |
| B39 | AC1 | 75/25 | YES | YES |
| B40 | AB1 | 63/37 | YES | YES |
| B41 | AA3 | 63/37 | YES | YES |
| B42 | AB2 | 63/37 | YES | YES |
| B43 | AB3 | 63/37 | YES | YES |
| B44 | AB4 | 75/25 | YES | YES |
| B45 | AC1 | 75/25 | YES | YES |
| B46 | AA4 | 75/25 | YES | YES |
| B47 | AA3 | 75/25 | YES | YES |
| B48 | AA5 | 75/25 | YES | YES |
| B49 | AA6 | 75/25 | YES | YES |
| B50 | AA7 | 75/25 | YES | YES |
| B51 | AA9 | 75/25 | YES | YES |

### C Pharmacodynamics

### C0. Protocol for Measuring the Pharmacodynamics of the Insulin Solutions

Preclinical studies were carried out on pigs with a view to evaluating four compositions according to the invention:

Insulin glargine/insulin lispro (75/25), formulated with the compound AB1 (17 mg/ml) described in example B33.

Insulin glargine/insulin lispro (62.5/37.5), formulated with the compound AB1 (14 mg/ml) described in example B40.

Insulin glargine/insulin lispro (75/25), formulated with the compound AA5 (15 mg/ml) described in example B48.

Insulin glargine/insulin lispro (75/25), formulated with the compound AA3 (10 mg/ml) described in example B47.

The hypoglycemic effects of these compositions were compared to those obtained after simultaneous but separate injections of Lantus® (pH 4) and of a prandial insulin Humalog® (insulin lispro) in the proportions 75% of Lantus®/25% of Humalog®.

Ten domestic pigs weighing approximately 50 kg, previously catheterized at the level of the jugular, are deprived of food for 2.5 hours before the beginning of the experiment. In the hour preceding the injection of insulin, 3 blood samples are taken in order to determine the basal level of glucose.

The injection of insulin at a dose of 0.2, 0.3 or 0.4 IU/kg, according to the study, is carried out by subcutaneous injection in the neck, under the animal's ear, using the Novopen® junior insulin pen fitted with a 31 G needle.

Blood samples are then taken after 10, 20, 30, 40 and 50 minutes and 1, 1.5, 2, 3, 4, 5, 6, 7, 8, 9, 10, 11, 12, 13, 14, 15 and 16 hours. After taking each sample, the catheter is rinsed with a dilute heparin solution.

A drop of blood is taken to determine the blood glucose level by means of a glucometer.

The curves of mean glucose pharmacodynamics expressed as percentages of the basal level are represented in FIGS. 1, 2, 3 and 4.

FIG. 1: Curves of mean blood glucose level±standard error of the mean for the simultaneous administrations of Humalog® (100 IU/ml, 0.05 IU/kg) and Lantus® (100 IU/ml, 0.15 IU/kg) in comparison with the administration of a formulation according to the invention described in example B33 (400 IU/ml, 0.4 IU/kg).

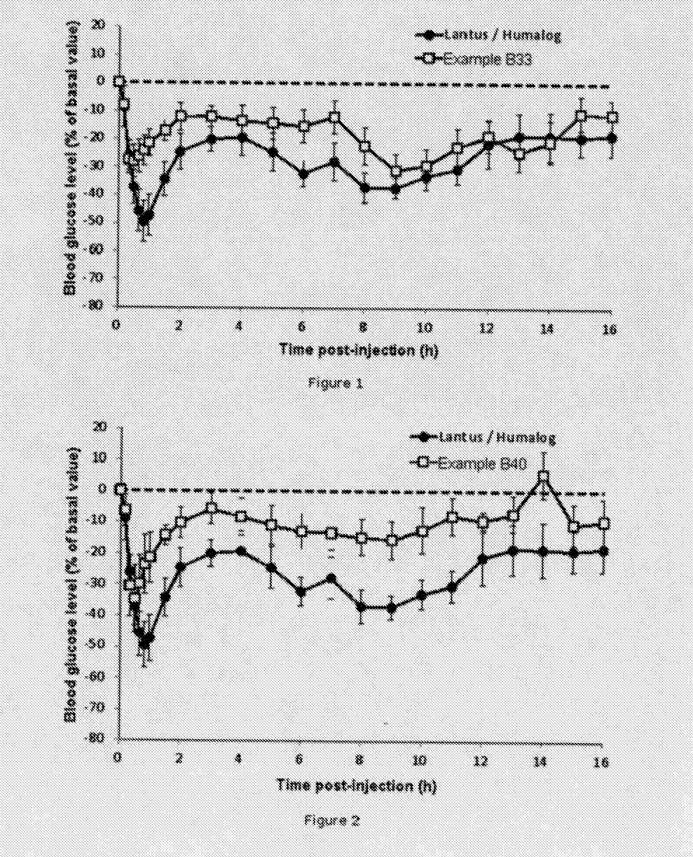

FIG. 2: Curves of mean blood glucose level±standard error of the mean for the simultaneous administrations of Humalog® (100 IU/ml, 0.05 IU/kg) and Lantus® (100 IU/ml, 0.15 IU/kg) in comparison with the administration of a formulation according to the invention described in example B40 (400 IU/ml, 0.4 IU/kg).

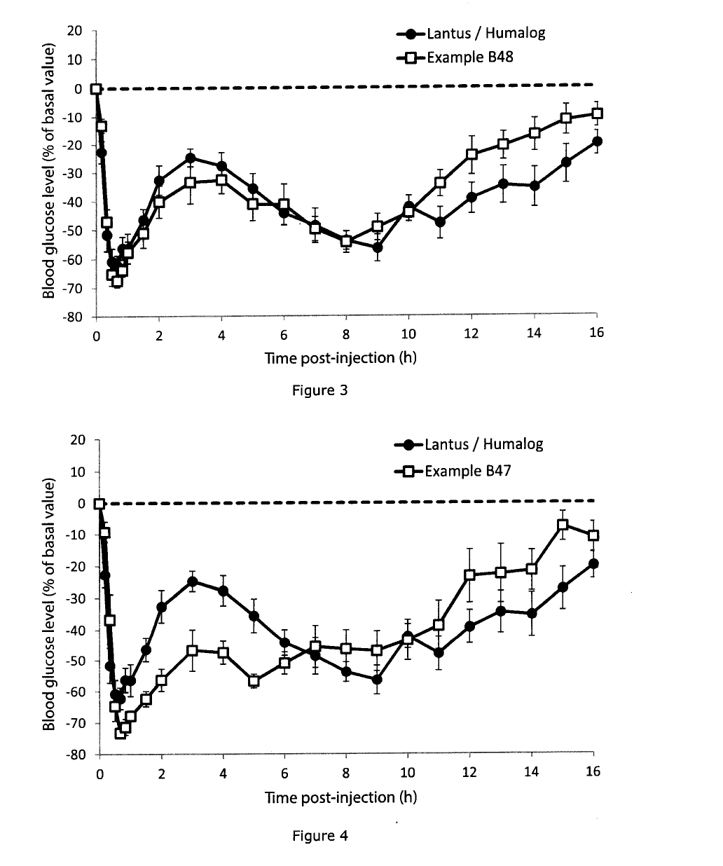

FIG. 3: Curves of mean blood glucose level±standard error of the mean for the simultaneous administrations of Humalog® (100 IU/ml, 0.075 IU/kg) and Lantus® (100 IU/ml, 0.225 IU/kg) in comparison with the administration of a formulation according to the invention described in example B48 (400 IU/ml, 0.3 IU/kg).

FIG. 4: Curves of mean blood glucose level±standard error of the mean for the simultaneous administrations of Humalog® (100 IU/ml, 0.075 IU/kg) and Lantus® (100 IU/ml, 0.225 IU/kg) in comparison with the administration of a formulation according to the invention described in example B47 (400 IU/ml, 0.3 IU/kg).

### C1. Results Regarding the Pharmacodynamics of the Solution of Insulin in Example B33

The pharmacodynamics results obtained with the simultaneous administrations of Humalog® and Lantus® in comparison with the formulation described in example B33 are presented in FIG. 1. The hypoglycemic activity of the formulation described in example B33 is two-phase. The rapid first phase is defined by a marked decrease in blood glucose level during the first 30 minutes, characteristic of the rapid effect of insulin lispro, indicating that the presence of the compound AB1 does not disrupt the fast-acting nature of Humalog®. This first phase is also visible on the Lantus®/Humalog® double injection. After 30 minutes, the blood glucose level increases again up to 2 hours, before a slower second phase, characterized by a hypo-glycemic activity which is less marked and sustained until 16 hours post-injection. This second phase is characteristic of the basal effect of insulin glargine, also visible on the double injection, indicating that it is indeed preserved with the formulation as claimed in the invention, described in example B33.

### C2. Results Regarding the Pharmacodynamics of the Solution of Insulin of Example B40

The pharmacodynamics results obtained with the simultaneous administrations of Humalog® and Lantus® in comparison with the formulation described in example B40 are presented in FIG. 2. The hypoglycemic activity of the formulation described in example B40 is two-phase. The rapid first phase is defined by a marked decrease in blood glucose level during the first 30 minutes, characteristic of the rapid effect of insulin lispro, indicating that the presence of the compound AB1 does not disrupt the fast-acting nature of Humalog®. This first phase is also visible on the Lantus®/Humalog® double injection. After 30 minutes, the blood glucose level increases again up to 2 hours, before a slower second phase, characterized by a hypo-glycemic activity which is less marked and sustained until 16 hours post-injection. This basal second phase is characteristic of the basal effect of insulin glargine, also visible on the double injection, indicating that it is indeed preserved with the formulation as claimed in the invention, described in example B40.

### C3. Results Regarding the Pharmacodynamics of the Solution of Insulin of Example B48

The pharmacodynamics results obtained with the simultaneous administrations of Humalog® and Lantus® in comparison with the formulation described in example B48 are presented in FIG. 3. The hypoglycemic activity of the formulation described in example B48 is two-phase. The rapid first phase is characterized by a marked decrease in blood glucose level during the first 30 to 45 minutes, similar to that induced by the Lantus®/Humalog® double injection, indicating that the presence of the compound AA5 does not disrupt the fast-acting nature of Humalog®. After 30 to 45 minutes, the blood glucose level increases again up to 2 to 3 hours, before a second phase, characterized by a hypoglycemic activity which is less marked and sustained up to 16 hours post-injection. This second phase is similar for the two formulations, indicating that the basal effect of insulin glargine is indeed preserved in the formulation as claimed in the invention, described in example B48.

### C4. Results Regarding the Pharmacodynamics of the Solution of Insulin of Example B47

The pharmacodynamics results obtained with the simultaneous administrations of Humalog® and Lantus® in comparison with the formulation described in example B47 are presented in FIG. 4. The hypoglycemic activity of the formulation described in example B47 is two-phase. The rapid first phase is characterized by a marked decrease in blood glucose level during the first 30 to 45 minutes (similar to that induced by the Lantus®/Humalog® double injection), indicating that the presence of the compound AA3 does not disrupt the fast-acting nature of Humalog®. After 30 to 45 minutes, the blood glucose level increases again up to 2 to 3 hours, before a second phase, characterized by a hypoglycemic activity which is less marked and sustained up to 16 hours post-injection for the two formulations, indicating that the basal effect of insulin glargine is indeed preserved in the formulation as claimed in the invention, described in example B47.

1. A composition in the form of an injectable aqueous solution, the pH of which is between 6.6 and 7.8, comprising at least:
a) a basal insulin, the isoelectric point pI of which is between 5.8 and 8.5;
b) an anionic compound bearing carboxylate charges and hydrophobic radicals, of formula I: 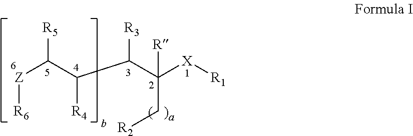
in which:
1) a=0 or 1;
2) b=0 or 1;
3) —X— is either a —C═O— radical, or a —CH₂— radical;
4) —Z— is either a —C═O— radical, or a —CH₂— radical;
5) —R₁ is chosen from the group consisting of the radicals:
i. —OH;
ii. -ƒ-[A]-COOH, then —X—═—CH₂—;
in which,
-A- is an at least divalent radical comprising from 1 to 15 carbon atoms comprising at least one heteroatom chosen from O, N and S, optionally bearing carboxyl or amine functions and/or -ƒ-[A]-COOH, comprising from 2 to 16 carbon atoms, is derived from an amino acid, from a diacid or from an alcohol acid and is bonded to the backbone of the molecule via a function ƒ;
ƒ is chosen from the group consisting of ether, ester, carbamate, amide or carbonate functions;
iii. -g-[B]-(k-[D])ₚ, then —X—═—CH₂—;
in which,
—B— is an at least divalent radical comprising from 1 to 15 carbon atoms comprising at least one heteroatom chosen from O, N and S, optionally bearing carboxyl or amine functions and/or -g-[B]-(k-)ₚ, comprising from 2 to 16 carbon atoms, is derived from an amino acid, from a diacid, from a dialcohol, from an alcohol acid, from a diamine or from an amine alcohol and is bonded to the backbone of the molecule via a function g and is bonded to at least one radical -D via a function k;
g is chosen from the group consisting of ether, ester, carbamate, amide and carbonate functions;
k is chosen from the group consisting of ester, amide, carbamate and carbonate functions;
p is a positive integer equal to 1 or 2;
said radical -D being a radical -[Hy] if p=2 or -[E]-(o-[Hy])ₜ if p=1; in which,
-E- is an at least divalent radical comprising from 1 to 15 carbon atoms comprising at least one heteroatom chosen from O, N and S, optionally bearing carboxyl or amine functions and/or k-[E]-(o-)ₜ, comprising from 2 to 16 carbon atoms, is derived from an amino acid, from a dialcohol, from a diamine, from a diacid or from an amine alcohol;
-Hy is a C₈ to C₃₀ linear or cyclic alkyl group or a C₈ to C₃₀ alkylaryl or arylalkyl, optionally substituted with one or more C₁ to C₃ alkyl groups, derived from a hydrophobic compound;
o is an ester, amide, carbamate or carbonate function;
t is a positive integer equal to 1 or 2;
k and o are identical or different;
iv. —NH-[E]-(o-[Hy])ₜ if —R′, —R₂, —R₃, —R₄, —R₅ and —R₆ are different from
-g-[B]-k-[D], —NH-[D] and —O-[D], in which
-E-, -Hy, o and t have the definitions given above;
v. —N(L)z-[E]-(o-[Hy])t if —R′, —R2, —R3, —R4, —R5 and —R6 are different from
-g-[B]-k-[D], —NH-[D] and —O-[D], and if —X—═—CH₂—, in which,
-D-, -Hy, -E-, o and t have the definitions given above;
z is equal to 1 or 2;
-L is chosen from the group consisting of:
—H and z is equal to 1, and/or
-[A]-COOH if ƒ is an ether function,
—CO-[A]-COOH and z is equal to 1, if ƒ is an ester function, and
—CO—NH-[A]-COOH and z is equal to 1 if ƒ is a carbamate function;
A has the meaning given above; and
viii. -A-, —B— or -E- which may be identical or different,
6) —R″ is chosen from the group consisting of the radicals:
i. —H, hydrogen atom, if b=1; and
ii. -k-[Hy] or -k-[E]-(o-[Hy])ₜ if b=0, a=0 and —R₁, —R₂, —R₃ different from -g-[B]-(k-[D])ₚ and —NH-[D];
-E- and t have the definitions given above;
-Hy is a C₁₂ to C₃₀ linear or cyclic alkyl group or a C₁₂ to C₃₀ alkylaryl or arylalkyl, optionally substituted with one or more C₁ to C₃ alkyl groups, derived from a hydrophobic compound;
7) —R₅ is chosen from the group consisting of the radicals:
i. —OH;
ii. -ƒ-[A]-COOH; and
iii. -g-[B]-(k-[D])ₚ;
in which:
-A-, —B—, -D- and p are defined as above,
ƒ, g and k, which may be identical or different, are defined as above;
8) —R₃ or —R₄, which may be identical or different, are chosen from the group consisting of the radicals:
i. —OH;
ii. -ƒ-[A]-COOH; and
iii. -g-[B]-(k-[D])ₚ;
-A-, —B—, -D-, p, ƒ, g and k being defined as above;
and/or
—R₆ is chosen from the group consisting of the radicals:
i. —OH;
ii. -ƒ-[A]-COOH if —Z—═—CH₂—;
iii. -g-[B]-(k-[D])ₚ if —Z—═—CH₂—;
iv. —O-[D] if —Z—═—C═O—; and
v. —NH-[D] if —Z—═—C═O—;
-A-, —B—, -D-, p, ƒ, g and k being defined as above;
and/or
—R₂ is chosen from the group consisting of the radicals:
i. —OH;
ii. -ƒ-[A]-COOH;
iii. -g-[B]-(k-[D])ₚ;
iv. —NH—COCH₃;
v. —NH₂; and
vi. —NH-[D];
-A-, —B—, -D-, p, ƒ, g and k being defined as above;
and/or
at most one of —R₂, —R₃, —R₄ and —R₆ is a backbone formed from a discrete number u of between 1 and 7 (1≦u≦7) of identical or different saccharide units linked via identical or different glycosidic linkages, said saccharide units being chosen from the group consisting of:
i. hexoses in cyclic form or in open reduced form,
ii. uronic acids in cyclic form or in open oxidized form,
iii. hexosamines in cyclic form, in open reduced form or in open oxidized form, and
iv. N-acetylhexosamines in cyclic form or in open reduced form, and
at least one of said saccharide units is substituted with at least one substituent —R′ chosen from:
-ƒ-[A]-COOH and/or
-k-[D] or g-[B]-(k-[D])ₚ, in which
-A-, —B—, -D-, p, ƒ, g and k are defined as above; and
when —R₁ is —NH-[E]-(o-[Hy])ₜ or —N(L)_{z}[E]-(o-[Hy])ₜ then —R₂, —R₃, —R₄, —R₅ and —R₆ are different from -g-[B]-(k-[D])ₚ, —O-[D] or
—NH-[D] and —R′ is different from -k-[D] or -g-[B]-(k-[D])ₚ;
and
9) the asymmetric carbon atoms are of absolute configuration R or S; the free acid functions being in the form of salts of alkali metal cations chosen from the group consisting of Na⁺ and K⁺.

2. The composition according to claim 1, wherein the anionic compound bearing carboxylate charges and hydrophobic radicals is chosen from the compounds of formula I in which the radical -ƒ-[A]-COOH is chosen from the radicals of formula VI below: 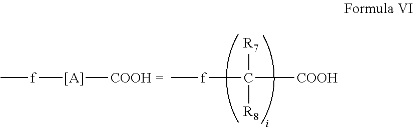
in which:
i is greater than or equal to 1 and less than or equal to 12, and
R₇ and R₈, which may be identical or different, are chosen from the group consisting of a hydrogen atom, a saturated or unsaturated, linear, branched or cyclic C₁ to C₆ alkyl, a benzyl, an alkylaryl, optionally comprising heteroatoms chosen from the group consisting of O, N and/or S, or functions chosen from the group consisting of carboxylic acid, amine, alcohol and thiol functions,
-ƒ-[A]-COOH comprises from 2 to 16 carbon atoms.

3. The composition according to claim 1, wherein the anionic compound bearing carboxylate charges and hydrophobic radicals of formula I, in which a=1, b=0 and —X— is a radical —CH₂—, is a compound chosen from the compounds of formula II: 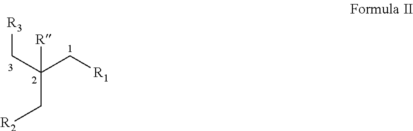
in which:
1) —R1, —R2 and —R3, which may be identical or different, are radicals -ƒ-[A]-COOH,
2) —R″ is either -k-[Hy], or -k-[E]-(o-[Hy])ₜ,
3) ƒ, k and o being identical or different,
the free acid functions being in the form of salts of alkali metal cations chosen from the group consisting of Na⁺ and K⁺.

4. The composition according to claim 1, wherein the anionic compound bearing carboxylate charges and hydrophobic radicals is a compound chosen from the compounds of formula I in which a=0, b=1, —X— is a radical —C═O—, —R″ is a hydrogen atom and —R₁ is a radical —NH-[E]-(o-[Hy])ₜ of formula III: 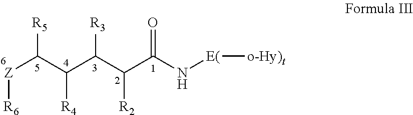
in which:
1) —R₂, —R₃, —R₄ and —R₅, which may be identical or different, are radicals -ƒ-[A]-COOH, and
2) —R₆ is a radical -ƒ-[A]-COOH if —Z—═CH₂ or a radical chosen from the group consisting of —O-[D] and —NH-[D] if —Z—═—C═O—;
3) ƒ and o being identical or different,
the free acid functions being in the form of salts of alkali metal cations chosen from the group consisting of Na⁺ and K⁺.

5. The composition according to claim 4, wherein the anionic compound bearing carboxylate charges and hydrophobic radicals is a compound chosen from the compounds of formula III in which Z═CH₂, of formula VIII: 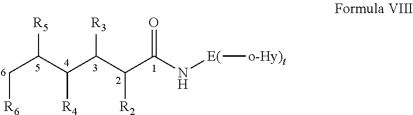
1) —R₂, —R₃, —R₄, —R₅ and —R₆, which may be identical or different, are radicals -ƒ-[A]-COOH,
2) -ƒ-[A]-COOH, comprising from 2 to 6 carbon atoms, is derived from an amino acid,
3) ƒ is a carbamate function,
4) ƒ and o being identical or different,
5) the free acid functions being in the form of salts of alkali metal cations chosen from the group consisting of Na⁺ and K⁺.

6. The composition according to claim 1, wherein the anionic compound bearing carboxylate charges and hydrophobic radicals of formula I, in which a=0, b=1, —X— is a radical —CH₂— and —R″ is a hydrogen atom, is a compound chosen from the compounds of formula IV: 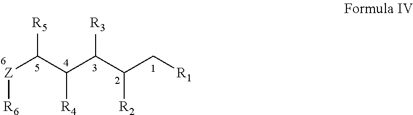
in which:
1) —R₅ is chosen from the group consisting of the radicals —OH, -ƒ-[A]-COOH and -g-[B]-(k-[D])ₚ,
2) —R₁ is chosen from the group consisting of the radicals —OH, -ƒ-[A]-COOH and -g-[B]-(k-[D])ₚ,
3) at most one of —R₂, —R₃, —R₄ and —R₆ is a backbone formed from a discrete number u of between 1 and 7 (1≦u≦7) of identical or different saccharide units linked via identical or different glycosidic linkages, at least one saccharide unit being chosen from the group consisting of:
i. hexoses in cyclic form or in open reduced form,
ii. uronic acids in cyclic form or in open oxidized form,
iii. hexosamines in cyclic form, in open reduced form or in open oxidized form, and
iv. N-acetylhexosamines in cyclic form, or in open reduced form,
at least one of said saccharide units being substituted with at least one substituent —R′=-ƒ-[A]-COOH, and/or at least one substituent —R′, which may be identical or different, chosen from the group consisting of -k-[D] and -g-[B]-(k-[D])ₚ;
-A-, —B—, -D-, ƒ, g and k being defined as above;
and/or
—R₃ and —R₄, which may be identical or different, are chosen from the group consisting of the radicals:
i. —OH;
ii. -ƒ-[A]-COOH; and
iii. -g-[B]-(k-[D])ₚ;
and/or
—R₂ is chosen from the group consisting of the radicals:
i. —OH;
ii. -ƒ-[A]-COOH;
iii. -g-[B]-(k-[D])ₚ;
iv. —NH—COCH₃;
v. —NH₂; and
vi. —NH-[D];
and/or
—R₆ is chosen from the group consisting of the radicals:
i. —OH;
ii. -ƒ-[A]-COOH if —Z—═—CH₂—;
iii. -g-[B]-(k-[D])ₚ if —Z—═—CH₂—;
iv. —O-[D], if —Z—═—C═O—; and
v. —NH-[D], if —Z—═—C═O—;
4) ƒ, g, k and o being identical or different;
5) the free acid functions being in the form of salts of alkali metal cations chosen from the group consisting of Na⁺ and K⁺;
6) the degree of substitution with carboxylate charges per saccharide unit is greater than or equal to 0.4; and
7) the degree of substitution with hydrophobic radicals per saccharide unit is less than or equal to 0.5.

7. The composition according to claim 6, wherein the anionic compound bearing carboxylate charges and hydrophobic radicals is chosen from the compounds of formula IV in which the radical -g-[B]— is chosen from the radicals of formula VII below: 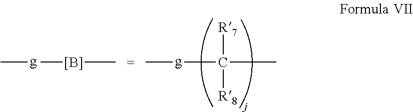
in which:
j is greater than or equal to 1 and less than or equal to 12, and
—R′7 and —R′8, which may be identical or different, are chosen from the group consisting of a hydrogen atom, a saturated or unsaturated, linear, branched or cyclic C₁ to C₆ alkyl, a benzyl, and an alkylaryl, optionally comprising heteroatoms chosen from the group consisting of O, N and/or S, or functions chosen from the group consisting of carboxylic acid, amine, alcohol and thiol functions,
-g-[B]— comprises from 1 to 15 carbon atoms.

8. The composition according to claim 6, wherein the anionic compound bearing carboxylate charges and hydrophobic radicals of formula IV, in which Z═CH₂, p=1 and t=1, is a compound chosen from the compounds of the formula IX: 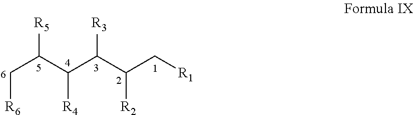
in which:
1) R₁, R₂, R₃, R₄, R₅ and R₆ are defined as above,
2) D is a radical -[E]-o-[Hy],
3) -E- and o are defined as above,
4) -Hy is a C₂₀ to C₃₀ linear or cyclic alkyl group or a C₂₀ to C₃₀ alkylaryl or arylalkyl, optionally substituted with one or more C₁ to C₃ alkyl groups, which is derived from a hydrophobic compound,
5) ƒ, g, k and o being identical or different,
6) the free acid functions being in the form of salts of alkali metal cations chosen from the group consisting of Na⁺ and K⁺,
7) the degree of substitution with carboxylate charges per saccharide unit is greater than or equal to 0.4,
8) the degree of substitution with hydrophobic radicals per saccharide unit is less than or equal to 0.5.

9. The composition according to claim 6, wherein the anionic compound bearing carboxylate charges and hydrophobic radicals of formula IV, in which Z═CH₂, p=1 and t=2, is chosen from the compounds of formula X: 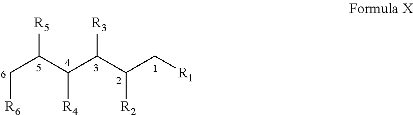
in which:
1) R₁, R₂, R₃, R₄, R₅ and R₆ are defined as above,
2) D is a radical -[E]-(o-[Hy])₂,
3) -k-E-(o-)₂, comprising 2 to 6 carbon atoms, are derived from a trivalent amino acid,
4) -Hy is a C₈ to C₂₀ linear or cyclic alkyl group or a C₈ to C₂₀ alkylaryl or arylalkyl, optionally substituted with one or more C₁ to C₃ alkyl groups, which is derived from a hydrophobic alcohol,
5) ƒ, g and k being identical or different,
6) o is an ester function,
7) the free acid functions being in the form of salts of alkali metal cations chosen from the group consisting of Na⁺ and K⁺,
8) the degree of substitution with carboxylate charges per saccharide unit is greater than or equal to 0.4,
9) the degree of substitution with hydrophobic radicals per saccharide unit is less than or equal to 0.5.

10. The composition according to claim 6, wherein the anionic compound bearing carboxylate charges and hydrophobic radicals of formula IV, in which Z═CH₂, p=1 and t=1, is a compound chosen from the compounds of formula XI: 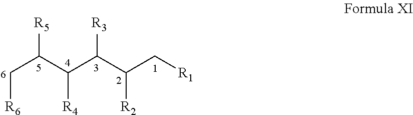
in which:
1) R₁, R₂, R₃, R₄, R₅ and R₆ are as defined above,
2) D is a radical -[E]-o-[Hy],
3) -k-E-o- are derived from a hydrophobic amino acid chosen from the group consisting of leucine, phenylalanine, isoleucine and valine, in their L, D or racemic forms,
4) -Hy is a C₈ to C₂₀ linear or cyclic alkyl group or a C₈ to C₂₀ alkylaryl or arylalkyl, optionally substituted with one or more C₁ to C₃ alkyl groups, which is derived from a hydrophobic alcohol,
5) ƒ, g and k being identical or different,
6) o is an ester function,
7) the free acid functions being in the form of salts of alkali metal cations chosen from the group consisting of Na⁺ and K⁺,
8) the degree of substitution with carboxylate charges per saccharide unit is greater than or equal to 0.4,
9) the degree of substitution with hydrophobic radicals per saccharide unit is less than or equal to 0.5.

11. The composition according to claim 1, wherein the anionic compound bearing carboxylate charges and hydrophobic radicals is a compound of formula I in which a=0, b=1 and R″ is a hydrogen atom, chosen from the compounds of formula V: 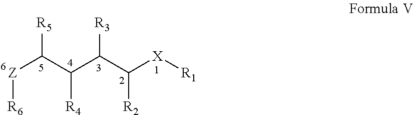
in which:
1) R₁ is either a radical —NH-[E]-(o-[Hy])ₜ, or a radical —N(L)_{z}-[E]-(o-[Hy])ₜ,
2) R₂, R₃, R₄ and R₆, which may be identical or different, are chosen from the group consisting of the radicals —OH and -ƒ-[A]-COOH,
and/or at most one of R₂, R₃, R₄ and R₆ is a backbone formed from a discrete number u of between 1 and 7 (1≦u≦7) of identical or different saccharide units substituted with at least one substituent R′=-ƒ-[A]-COOH,
3) R₅, which may be identical or different, are either a radical —OH, or a radical -ƒ-[A]-COOH,
4) the degree of substitution with carboxylate charges per saccharide unit is greater than or equal to 0.4,
5) ƒ and o being identical or different,
6) the free acid functions being in the form of salts of alkali metal cations chosen from the group consisting of Na⁺ and K⁺.

12. The composition according to claim 1, wherein the basal insulin, the isoelectric point of which is between 5.8 and 8.5, is insulin glargine.

13. The composition according claim 1, which comprises between 40 and 500 IU/ml of basal insulin, the isoelectric point of which is between 5.8 and 8.5.

14. The composition according to claim 1, wherein the concentration of polyanionic compound bearing carboxylate charges and hydrophobic radicals is at most 60 mg/ml.

15. The composition according to claim 1, which also comprises a prandial insulin.

16. The composition according to claim 15, wherein the prandial insulin is human insulin.

17. The composition according to claim 15, which in total comprises between 40 and 800 IU/ml of insulin with a combination of prandial insulin and basal insulin, the isoelectric point of which is between 5.8 and 8.5.

18. The composition according to claim 15, wherein the proportions between the basal insulin, the isoelectric point of which is between 5.8 and 8.5, and the prandial insulin are, as a percentage, 25/75, 30/70, 40/60, 50/50, 60/40, 63/37, 70/30, 75/25, 80/20, 83/17, or 90/10.

19. An anionic compound bearing carboxylate charges and hydrophobic radicals chosen from the compounds of formula I, in which a=1, b=0 and —X— is a radical —CH₂—: 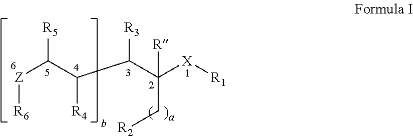
in which:
1) a=1, and
2) b=0, and
3) —X— is a radical —CH₂—; and
4) —R₁ is chosen from the group consisting of the radicals:
i. —OH; and
ii. -ƒ-[A]-COOH;
in which,
-A- is an at least divalent radical comprising from 1 to 15 carbon atoms comprising at least one heteroatom chosen from O, N and S, optionally bearing carboxyl or amine functions and/or -ƒ-[A]-COOH, comprising from 2 to 16 carbon atoms, is derived from an amino acid, from a diacid or from an alcohol acid and is bonded to the backbone of the molecule via a function ƒ;
ƒ is chosen from the group consisting of ether, ester, carbamate, amide or carbonate functions;
5) —R″ is chosen from the group consisting of the radicals:
i. -k-[Hy] or -k-[E]-(o-[Hy])ₜ
k is chosen from the group consisting of ester, amide, carbamate and carbonate functions;
-E- is an at least divalent radical comprising from 1 to 15 carbon atoms comprising at least one heteroatom chosen from O, N and S, optionally bearing carboxyl or amine functions and/or k-[E]-(o-)ₜ, comprising from 2 to 16 carbon atoms, is derived from an amino acid, from a dialcohol, from a diamine, from a diacid or from an amine alcohol;
o is an ester, amide, carbamate or carbonate function; and
t being a positive integer equal to 1 or 2;
k and o being identical or different;
-Hy is a C₁₂ to C₃₀ linear or cyclic alkyl group or a C₁₂ to C₃₀ alkylaryl or arylalkyl, optionally substituted with one or more C₁ to C₃ alkyl groups, which is derived from a hydrophobic compound;
6) —R₂ and —R₃ are chosen from the group consisting of the radicals:
i. —OH; and
ii. -ƒ-[A]-COOH;
-A- and ƒ being defined as above;
and
7) the assymetrical carbon atoms are of absolute configuration R or S; the free acid functions being in the form of salts of alkali metal cations chosen from the group consisting of Na⁺ and K⁺.

20. The anionic compound bearing carboxylate charges and hydrophobic radicals according to claim 19, which is chosen from the compounds of formula II: 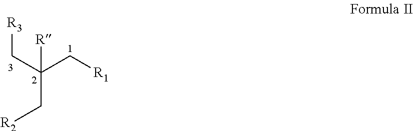
in which:
1) —R₁, —R₂ and —R₃, which may be identical or different, are radicals -ƒ-[A]-COOH,
2) —R″ is either -k-[Hy] or -k-[E]-(o-[Hy])ₜ,
3) ƒ, k and o being identical or different,
4) the free acid functions being in the form of salts of alkali metal cations chosen from the group consisting of Na⁺ and K⁺.

21. An anionic compound bearing carboxylate charges and hydrophobic radicals chosen from the compounds of formula I, in which a=0, b=1, —X— is a radical —C═O—, —R″ is a hydrogen atom and —R₁ is a radical —NH-[E]-(o-[Hy])ₜ: 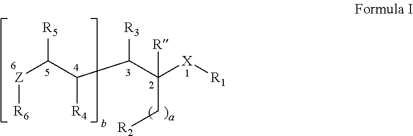
in which:
1) a=0,
2) b=1,
3) —X— is a radical —C═O—;
4) —Z— is either a radical —C═O— or a radical —CH₂—;
5) —R₂, —R₃, —R₄ and —R₅ are chosen from the group consisting of the radicals:
i. —OH; and
ii. -ƒ-[A]-COOH;
in which,
-A- is an at least divalent radical comprising from 1 to 15 carbon atoms comprising at least one heteroatom chosen from O, N and S, optionally bearing carboxyl or amine functions and/or -ƒ-[A]-COOH, comprising from 2 to 16 carbon atoms, is derived from an amino acid, from a diacid or from an alcohol acid and is bonded to the backbone of the molecule via a function ƒ;
ƒ is chosen from the group consisting of ether, ester, carbamate, amide and carbonate functions;
6) —R″ is a hydrogen atom,
7) —R₁ is a radical —NH-[E]-(o-[Hy])ₜ,
i. -E- is an at least divalent radical comprising from 1 to 15 carbon atoms comprising at least one heteroatom chosen from O, N and S, optionally bearing carboxyl or amine functions and/or NH-[E]-(o-)ₜ, comprising from 2 to 16 carbon atoms, is derived from an amino acid, from a diamine or from an amine alcohol;
ii. -Hy is a C₁₂ to C₃₀ linear or cyclic alkyl group or a C₁₂ to C₃₀ alkylaryl or arylalkyl, optionally substituted with one or more C₁ to C₃ alkyl groups, which is derived from a hydrophobic compound;
iii. o is an ester, amide, carbamate or carbonate function; and
iv. t being a positive integer equal to 1 or 2;
8) —R₆ is chosen from the group consisting of the radicals:
i. —OH;
ii. -ƒ-[A]-COOH if —Z—═—CH₂—;
iii. —O-[D] if —Z—═—C═O—;
iv. —NH-[D] if —Z—═—C═O—; and
v. the radical -D being -[E]-(o-[Hy])ₜ
-A-, -E-, ƒ, o and Hy being defined as above;
and
9) the assymetrical carbon atoms are of absolute configuration R or S; the free acid functions being in the form of salts of alkali metal cations chosen from the group consisting of Na⁺ and K⁺.

22. The anionic compound bearing carboxylate charges and hydrophobic radicals according to claim 21, which is chosen from the compounds of formula VIII: 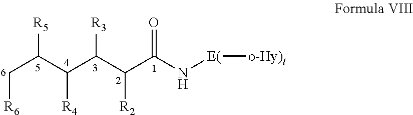
1) —R₂, —R₃, —R₄, —R₅ and —R₆, which may be identical or different, are radicals -ƒ-[A]-COOH,
2) -ƒ-[A]-COOH, comprising from 2 to 6 carbon atoms, is derived from an amino acid,
3) ƒ is a carbamate function,
4) ƒ and o being identical or different,
5) the free acid functions being in the form of salts of alkali metal cations chosen from the group consisting of Na⁺ and K⁺.

23. A single-dose formulation at a pH of between 7 and 7.8 comprising a basal insulin, the isoelectric point of which is between 5.8 and 8.5, and a prandial insulin.
